(19) United States
(12) Patent Application Publication
(10) Pub. No.: US 2025/0345190 A1
(43) Pub. Date: Nov. 13, 2025

(54) DRUG ELUTING STENT

(71) Applicant: SINO MEDICAL SCIENCES TECHNOLOGY INC., Tianjin, Hebei (CN)

(72) Inventors: Jianhua SUN, Irvine, CA (US); Christophe BUREAU, URIAGE (FR); Wenbin CAI, Tianjin, Hebei (CN); Tianzhu LI, Irvine, CA (US); Xiaoran KANG, Tianjin, Hebei (CN)

(21) Appl. No.: 18/259,979

(22) PCT Filed: Dec. 29, 2021
(86) PCT No.: PCT/IB2021/062445

§ 371 Date: Jun. 29, 2023

Related U.S. Application Data
(60) Provisional application No. 63/131,702, filed on Dec. 29, 2020.
(60) Provisional application No. 63/133,908, filed on Jan. 5, 2021.

(51) Int. Cl. A61F 2/82; A61K 9/00; A61K 31/436

(52) U.S. Cl. CPC A61F 2/82; A61K 9/0024; A61K 31/436; A61F 2210/0014; A61F 2240/001; A61F 2250/0067

(57) ABSTRACT

The present disclosure relates to drug eluting stents (DES), methods of making, using, and verifying long-term stability of the DES, and methods for predicting long term stent efficacy and patient safety after implantation of a DES. In one embodiment, a DES may include a stent framework; a drug-containing layer; a drug embedded in the drug-containing layer; and a biocompatible base layer disposed over the stent framework and supporting the drug-containing layer. The drug-containing layer may have an uneven coating thickness. In addition, or in alternative, the drug-containing layer may be configured to enable an instantaneous drug release which is maximum at a time where smooth muscle cells should have their maximum proliferation, to be essentially zero ca. 30 days after implantation, and to significantly dissolve/dissipate/disappear between 45 days and 90 days after stent implantation. Stents of the present disclosure focus on suppressing smooth muscle cells' over-proliferation, without hindering their normal growth, which may reduce, minimize, or eliminate patient risks associated with the implantation of a stent, including, for example, restenosis, thrombosis, and/or MACE.

### TECHNICAL FIELD

The present disclosure relates to drug eluting stents, methods of making and using the drug eluting stents, as well as methods for predicting long term stent efficacy and patient safety after implantation of a drug eluting stent. More specifically, and without limitation, the present disclosure relates to the design of a drug eluting stent comprising a stent framework (e.g., metal based or made with biodegradable materials) and a layer or layers covering all or part of the surface of said stent, capable of hosting a drug and releasing it in a sustained manner, in such a way that patient risks associated with the implantation of said drug eluting stent are minimized or eliminated. The stents disclosed herein are capable of enabling functional restoration of endothelial cell layers after implantation.

### BACKGROUND

Over the years, the use of coatings for medical devices and drug delivery has become a necessity, notably for augmenting the capabilities of medical devices and implants. Drug eluting medical devices have emerged as a leading biomedical device for the treatment of cardiovascular disease.

Heart disease and heart failure are two of the most prevalent health conditions in the U.S. and the world. In coronary artery disease, the blood vessels in the heart become narrow. When this happens, the oxygen supply is reduced to the heart muscle. A primary treatment of coronary artery disease was initially done by surgery, e.g., CABG (Coronary Artery Bypass Graft), which are normal and efficient procedures performed by cardiac surgeons. The mortality and morbidity, however, were rather high.

In the 1960s, some physicians developed a less invasive treatment by using medical devices. These devices were inserted through a small incision at the femoral artery. For example, balloon angioplasty (which may be used to widen an artery that has become narrowed using a balloon catheter which is inflated to open the artery and is also termed PTCA (Percutaneous Transluminal Coronary Angioplasty)) is used in patients with coronary artery disease. Following balloon angioplasty, approximately 40 to 50% of coronaries arteries are generally affected by restenosis (the re-narrowing of a blood vessel after it has been opened, usually by balloon angioplasty), usually within 3 to 6 months due to either thrombosis (the development of a blood clot in the vessels which may clog a blood vessel and stop the flow of blood) or abnormal tissue growth. As a result, restenosis constitutes one of the major limitations to the effectiveness of PTCA.

The introduction of the bare metal stent (BMS) in the late 1980s, when used to keep coronary arteries expanded, partially alleviated this problem, as well as that of the dissections of arteries upon balloon inflation in the PTCA procedure.

Some of the stents are a mesh tube mounted on a balloon catheter (e.g., a long thin flexible tube that can be inserted into the body). In some examples, the stents are threaded to the heart. However, the BMS initially continued to be associated with a general restenosis rate of around 25% of patients affected 6 months after stent insertion. Usually, stent struts end up embedded by the arterial tissue in growth. This tissue is typically made of smooth muscle cells (SMCs), the proliferation of which may be provoked by the initial damaging of the artery upon stent apposition.

As depicted in FIG. 1, the whole inner surface of the vessel (100) is covered by “active” of functional ECs (101), i.e. endothelial cells spontaneously producing nitrogen oxide (NO), a small molecule acting as a signal to stop the proliferation of SMCs (103) underneath. This natural release of NO by ECs (101) takes place generally when ECs (101) are in immediate contact to one another, e.g., paving the inner surface of the artery by a continuous and closely packed film.

When a stent (or a balloon) is inflated inside a vessel (150), stent struts in contact with the vessel walls will partly destroy the EC layer and injure the artery, e.g. at contact points (105a) and (105b). FIG. 2. The natural release of NO is thus—at least locally at contact points (105a) and (105b)—highly perturbed. This injury may trigger the proliferation of SMCs as a repair mechanism, e.g., SMCs (107a) and (107b). The uncontrolled proliferation of SMCs may cause the re-closing of the vessel, or “re-stenosis.” If, while SMCs (107a) and (107b) are proliferating, ECs (101) can also proliferate and eventually cover again the stent struts and SMCs (107a) and (107b) via a continuous film, then the NO release may be restored and the proliferation of SMC's may be stopped. Consequently, the risk of restenosis may be lessened (if not eliminated) and the situation may stabilize.

One of the biggest challenges of the interventional cardiology industry since the 1990s has been to first understand and then secure this “race” for complete EC coverage and restoring the EC layer functions. The endothelium is a monolayer of cells lining the inside of all blood and lymph vasculature. One important function of the endothelium is to regulate the movement of fluid, macromolecules, and white blood cells between the vasculature and the interstitial tissue. This is mediated, in part, by the ability of endothelial cells to form strong cell-cell contacts by using a number of transmembrane junctional proteins, including VE-Cadherin and p120-catenin. Colocalization of the two proteins is an indication of a well-functioning endothelial cell layer.

Two strategies have been historically considered to restore an artery following stent implantation. One goal of some Drug Eluting Stents (DES) designs is to promote the proliferation of active endothelial cells (ECs) to accelerate their migration and eventual coverage of the surface of the stent. If these new ECs are active, e.g., form a continuous and close packed film, they usually spontaneously release NO and thereby hinder the proliferation of SMCs.

Another goal of some DES designs is to inhibit the proliferation of smooth muscle cells (SMCs). Generally, this has been targeted via the local release of an anti-proliferative agent (usually an anti-angiogenesis drug, similar to anti-cancer agents) from the surface of the stent.

Many DES on the market are made on the basis of a polymeric release matrix from which the drug is eluted. First and second generation stents were often coated with a biostable polymer. In such stents, the polymer stays permanently on the stent, and is generally assumed to have little effect both on the inflammatory response and the proliferation of ECs. In some cases, however, these stents do not release 100% of the drug that their coating is hosting. In particular, sometimes the majority of the drug remains in the polymer coating for long periods of time. Furthermore, most drugs used so far are not selective and tend to inhibit the proliferation of ECs more than that of SMCs. Most DES on the market are such that drug release reaches completion about 3 months or more after stent implantation. A relative long term of presence of drug will reduce the growth rate of cells and result in poor quality of restoration of functional of newly formed endothelium.

This drawback may have dramatic and potentially lethal consequences for the patients and, thus, for the DES industry. Indeed, despite the possible reduction in restenosis from ca. 20% with Bare Metal Stents (BMS) to ca. 5% with Drug Eluting Stents (DES) in the first year, the massive introduction of DES brought two new challenges: (1) the phenomenon of late thrombosis, i.e., thrombosis happens one year or more after stent implantation, and (2) progressive growth of the neo-intimal layer leading to restenosis again. Accordingly, what DES has generally accomplished is to delay the occurrence of restenosis yet cause other complications, such as thrombosis, neoatherosclerosis in the years after the DES implantation.

The implantation of bare metal stents is understood to be a source of thrombosis, in addition to restenosis, but the former is generally handled by a systemic Dual Anti-Platelet Therapy (DAPT) associating two anti-thrombotic agents, e.g., aspirin and clopidogrel. For example, patients in whom a stent was implanted were often prescribed such DAPT for 1 to 2 months. With drug eluting stents, numerous cases of re-clotting of the artery due to coagulation (thrombosis) after interruption of the DAPT have been reported. Accordingly, many cardiologists maintain the DAPT for 3, 6, 9 and now 12 months or more. By 2005-2006, several cases were reported that myocardial infarction with total stent thrombosis may occur only a couple of weeks after interruption of an 18-month DAPT.

Late thrombosis is an abrupt complication which can be lethal when occurring if the patient is not under medical follow-up or—even if the patient is—while the patient is away from the cathlab or from an adequately equipped medical centre. Moreover, DAPT may present a bottleneck that is difficult to manage, as some patients may decide by themselves to stop it after a period of use, or forget to have their medicines refilled or to take their medicines, or may have to undergo a clinical intervention which could not be anticipated, and are thus in the position to have to stop the anti-thrombotic treatment.

The exact causes of late thrombosis still are not fully understood. Pathologists estimate that late thrombosis reveals an incomplete coverage of the stent by ECs, leaving metallic or polymeric materials in contact with the blood over prolonged periods, on which platelet adhesion is likely to occur, which may lead to catastrophic precipitation of a thrombus. Alternative interpretations propose that the incomplete coverage by ECs may be the result of the incomplete release of the drug from the release layer, which may inhibit the proliferation of ECs in their attempt to migrate and cover the surface of said polymer+drug+SMC layer.

The thickness of the stent struts may further present a source of hindrance of the proliferation of ECs. Whenever ECs have to proliferate on a surface, the rate of their proliferation is often negatively (and largely) influenced by the height of obstacles that they have to overcome on this surface towards complete coverage. Accordingly, not all stent designs and drug release profiles are equal. For example, when the DES is apposed in the artery, the injured EC layer has to overcome obstacles with a height roughly equal to the thickness of the stent strut+the thickness of the drug release polymer layer+the thickness of the SMC layer which has started to form. The former two thicknesses are related to the design of the DES, while the latter thickness is linked to the efficacy of the drug, its loading in the release layer, and its release rate. Thus, a need still exists for developing a new stent and method of making a stent that can decrease patient risks associated with the implantation of stents (e.g., restenosis, thrombosis, MACE).

### SUMMARY

The present disclosure relates to drug eluting stents, as well as methods of making and using the drug eluting stents, and a method of predicting stent efficacy and patient safety. In one embodiment, the drug eluting stent (1) combines four parts: a stent framework (2), a drug-containing layer (3), a drug (4), and a biocompatible base layer supporting the drug-containing layer (5). In one embodiment, the stent and the method of making the stent are designed so as to manipulate the time to achieve a sufficient neointima coverage of the stent surface/vascular wall and improve endothelium function restoration by, for example, manipulating the thickness of the drug-containing layer and the distribution of that thickness, and/or the pharmacokinetics of drug delivery to the arterial wall surrounding the stent. The neointima formed above the implanted stent strut typically includes smooth muscle cells, matrix, and monolayer of endothelial cells. It was discovered that an 80%-90% neointima coverage of the stent struts by about 30 days post-stent implantation correlates with and is predictive of lower side effects by 1 year or later post-stent implantation, relative to stents with lower percentage of neointima coverage. This discovery provides an end parameter or guide for stent design whereby one or more physical features of a stent can be designed so as to result in a stent that offers 80%-90% neointima coverage of the stent struts by about 30 days, which is an early predictor of stent performance at 1-year post-stent implantation. Prior to this discovery, it was not known that such an early parameter post-stent implantation could be used to predict stent performance and serve as a goal for stent design. In one embodiment, this coverage is achieved by designing the stent such that the thickness of the drug-containing layer in the luminal side is different from the thickness in the abluminal side of the stent. In other embodiments, this coverage is achieved by designing a stent with a specific drug delivery profile into the arterial area of the stent. In another embodiment, it was discovered that a superior stent is achieved by designing a stent with specific levels of Evans-Blue staining and/or VE-Cadherin/p120 co-localization at 45- and 90 days post-stent implantation in a rabbit stent implantation model. In another embodiment, it was discovered that a superior stent is achieved by designing a stent with a specific cell shape index at specific time points after stent implantation (e.g., 45 and 90 days in a rabbit stent implantation model). In one embodiment, the stents of the disclosure minimize late thrombosis, i.e. re-clotting of the artery one year or more after stent implantation and progressive thickness of the neo-intimal layer leading to restenosis again. In one embodiment, the stent and the method of making the stent are such that they reduce the number or frequency of major adverse cardiac events (MACE). In one embodiment, the stent is designed to promote a high percentage (e.g., 80-90%) of neointimal coverage of the surface of stent struts within 30 days, which unexpectedly significantly improves strength efficacy and patient safety.

In one embodiment, the stent framework (2) may be fabricated from a single (or more) pieces of metal or wire or tubing. For example, the stent framework may comprise cobalt-chromium (e.g., MP35N or MP20N alloys), stainless steel (e.g., 316L), nitinol, tantalum, platinum, titanium, suitable biocompatible alloys, other suitable biocompatible materials, and/or combinations thereof.

In some embodiments, the stent framework (2) may be biodegradable. For example, the sent framework (2) may be fabricated from magnesium alloy, Zinc alloy, iron alloy, polylactic acid, polycarbonate polymers, salicylic acid polymers, and/or combinations thereof. In other words, an example is any biocompatible but also biodegradable materials that can be fabricated in such way that the radical force is sufficiently strong to be implantable and support to stabilize the lesion and vessel retraction, but where the thickness of the stent is less than 120 um.

In other embodiments, the stent framework (2) may be fabricated from one or more plastics, for example, polyurethane, teflon, polyethylene, or the like.

A drug-containing layer (3) may be made from polymers and may comprise a layer or layers covering all or part of the stent surface. Furthermore, a drug-containing layer (3) may be capable of hosting a drug (4) and releasing the drug (4) in a sustained manner.

In one embodiment, the drug-containing layer may have an uneven coating thickness. For example, a thickness of the drug-containing layer on a luminal side of the stent and/a thickness of the drug-containing layer on a lateral side of the stent is less than a thickness of the drug-containing layer on an abluminal side of the stent. In another example, a thickness of the drug-containing layer on a abluminal side of the stent and a thickness of the drug-containing layer on a lateral side of the stent is less than a thickness of the drug-containing layer on an luminal side of the stent.

In one embodiment, for example on account of the uneven coating thickness, the drug-containing layer may release the drug within 30 days of implantation within a vessel. The release time may be verified, for example, using a standard animal PK (Phanmaco-Kinetic) study. Accordingly, when the drug eluting stent (1) is implanted into the human body vessel, the drug (4) may be released from coating (3) within 30 days or less. In other embodiments, the drug is released at different rates, such as 45 days or less, 60 days or less, or any interval in between, such as, for example, between 30 and 45 days, between 45 days and 60 days, and any other combination of intervals.

In some embodiments, the drug may be included only on an abluminal side of the stent. In some embodiments, the drug may be included only on a lateral side of the stent

In embodiments where the drug-containing layer (3) is made from a bio-degradable or bio-absorbable polymer/s, the polymer(s) may be bio-degraded or bio-absorbed between day 15 and day 30, day 30 and day 45, and day 45 day and day 60 of implantation of the stent. In other embodiments, the polymer/polymers is/are bio-degraded or bio-absorbed within, such as, 30 days or less, 45 days or less, 60 days or less, and any interval in between, such as, for example, between 15 and 30 days, 30 and 45 days, between 45 days and 60 days, and any other combination of intervals.

In some embodiments, the polymer on a luminal side and/or a lateral side of the stent may differ from the polymer on an abluminal side. For example, one or more polymers forming the drug-containing layer on a luminal side of the stent and the drug-containing layer on a lateral side of the stent degrade faster than one or more polymers forming the drug-containing layers on an abluminal side of the stent. The biocompatible base layer (5) may be formed over the stent framework (2) and may have a more biocompatible surface than the stent framework (2). For example, the biocompatible base layer (5) may be made from poly n-butyl methacrylate, poly-methyl methacrylate, poly-acrylic acid, poly-N-[Tris(hydroxymethyl)-methyl]-acrylamide (poly-NTMA), PEDOT (poly(3,4-ethylenedioxythiophene)) PTFE, PVDF-HFP, poly(styrene-b-isobutylene-b-styrene), Parylene C, Poly-DopAmine (PDA), PVP, PEVA, SBS, PC, TiO2 or any material that has good biocompatibility (or combinations thereof).

In some embodiments, the biocompatible base layer is obtained from a pre-made polymer which is deposited by spray or by dipping.

In yet other embodiments, the biocompatible base layer is obtained by electrochemical processes from precursor molecules, and in particular precursor monomers, like electro-polymerization of conductive polymers like PEDOT (poly(3,4-ethylenedioxythiophene)), or electro-grafting of vinylic monomers or of aryl diazonium compounds.

In some embodiments, the biocompatible base layer may be selected to accelerate the healing of areas of the artery that were wounded during stent implantation, in particular to accelerate the migration of endothelial cells on its surface. Examples of such base layers include but not limit to electro-grafted poly-butyl methacrylate (see ref: link.springer.com/article/10.1007/s13239-021-00542-x) or electro-grafted poly-N-[Tris(hydroxymethyl)-methyl]-acrylamide.

In yet further embodiments, the biocompatible base layer may be selected to inhibit the production of inflammation markers from the stent surface and in particular inflammatory cytokines (IL-6, IL-8) or glycoproteins enabling the adhesion and local recruitment of leukocytes (E-selectin), while preserving or even boosting the production of thrombosis inhibitors such as Tissue Factor Pathway Inhibitor (TFPI), or Poly-DopAmine (PDA) (see ref: doi.org/10.1093/eurheartj/ehab027).

The following are some further exemplary embodiments of this disclosure:
1. A drug eluting stent, comprising at least four parts:
   a stent framework;
   a drug-containing layer;
   a drug embedded in the drug-containing layer; and
   a biocompatible base layer disposed over the stent framework and supporting the drug-containing layer,
   wherein one or more parts of the stent are designed to achieve a pre-designed drug release pharmacokinetic profile selected from:
(1) the drug pharmacokinetic profile has Tmax and Cmax (expressed in ng of drug per g of the artery tissue after implantation) so that:
(a) Tmax is between 400 and 600 hr, preferably 500 hr,
(b) Cmax is between 5 and 15 ng/g, preferably 10 ng/g, and/or
(c) the drug pharmacokinetic profile overlaps with the kinetic profile for smooth muscle cell proliferation post-stent implantation, as depicted in FIG. 16B, preferably wherein the arterial tissue concentration at the site of stent implantation peaks between 15 and 25 days, preferably at 20 days, and then decreases to allow for vascular restoration; and
   (2) the drug has a pharmacokinetic profile in the arterial tissue at the site of stent implantation about as depicted in FIG. 16A or FIG. 16B, optionally, wherein the drug is sirolimus.
2. The drug eluting stent of embodiment 1, wherein the drug is embedded essentially on the drug-containing layer on an abluminal side of the stent.
3. The drug eluting stent of any one of embodiments 1 through 2, wherein the stent framework is fabricated from a single piece of metal, wire, or tubing.
4. The drug eluting stent of embodiment 3, wherein the metal comprises at least one of stainless steel, nitinol, tantalum, cobalt-chromium MP35N or MP20N alloys, platinum, and titanium.
5. The drug eluting stent of any one of embodiments 1 through 3, wherein the stent framework is fabricated from a biodegradable material, such as a metallic alloy made from magnesium, zinc or iron.
6. The drug eluting stent of any one of embodiments 1 through 5, wherein the drug comprises at least one of an antithrombogenic agent, an anticoagulant, an antiplatelet agent, an antineoplastic agent, an antiproliferative agent, an antibiotic, an anti-inflammatory agent, a gene therapy agent, a recombinant DNA product, a recombinant RNA product, a collagen, a collagen derivative, a protein analog, a saccharide, a saccharide derivative, an inhibitor of smooth muscle cell proliferation, a promoter of endothelial cell migration, proliferation, and/or survival, and combinations of the same.
7. The drug eluting stent of embodiment 6, wherein the drug comprises sirolimus and/or a derivative or analog of sirolimus.
8. The drug eluting stent of embodiment 1, wherein the drug-containing layer has a thickness between 5 and 12 μm or 2-20 μm, preferably 1, 2, 3, 4, 5, 6, 7, 8, 9, 10, 11, 12, 13, 14, 15, 16. 17, 18, 19, or 20 μm in either the luminal, abluminal, or both sides.
9. The drug eluting stent of embodiment 1, wherein the drug-containing layer is selected from the group consisting of poly(hydroxyalkanoates) (PHAs), poly(ester amides) (PEAs), poly(hydroxyalkanoate-co-ester amides), polyacrylates, polymethacrylates, polycaprolactones, poly(ethylene glycol)(PEG), poly(propylene glycol)(PPG), poly(propylene oxide) (PPO), poly(propylene fumarate) (PPF), poly(D-lactide), poly(L-lactide), poly(D,L-lactide), poly(meso-lactide), poly(L-lactide-co-meso-lactide), poly(D-lactide-co-meso-lactide), poly(D,L-lactide-co-meso-lactide), poly(D,L-lactide-co-PEG), poly(D,L-lactide-co-trimethylene carbonate), poly(lactide-co-glycolide), poly(glycolic acid-co-trimethylene carbonate), poly(trimethylene carbonate), PHA-PEG, PBT-PEG (PolyActive®), PEG-PPO-PEG (Pluronic®), and PPF-co-PEG, polycaprolactones, polyglycerol sebacate, polycarbonates, biopolyesters, polyethylene oxide, polybutylene terephalate, polydioxanones, hybrids, composites, collagen matrices with grouth modulators, proteoglycans, glycosaminoglycans, vacuum formed small intestinal submucosa, fibers, chitin, dexran and mixtures thereof.
10. The drug eluting stent of embodiment 1, wherein the drug-containing layer is selected from tyrosine derived polycarbonates.
11. The drug eluting stent of embodiment 1, wherein the drug-containing layer is selected from poly(β-hydroxyalcanoate)s and derivatives thereof.
12. The drug eluting stent of embodiment 1, wherein the drug-containing layer comprises a polylactide-co-glycolide 50/50 (PLGA) or Poly Butyl MethAcrylate.
13. The drug eluting stent of embodiment 1, wherein the biocompatible base layer comprises at least one of poly poly-butyl methacrylate, poly-N-[Tris(hydroxymethyl)-methyl]-acrylamide (poly-NTMA), Poly-dopamine, PEDOT, PTFE, PVDF-HFP, poly(styrene-b-isobutylene-b-styrene), Parylene C, PVP, PEVA, SBS, PC, or TiO2.
14. The drug eluting stent of embodiment 1, wherein the biocompatible base layer comprises an electro-grafted layer, optionally an electro-grafted polymeric layer, optionally interdigitating with the drug-containing layer.
15. The drug eluting stent of embodiment 1, wherein the biocompatible base layer comprises an organic layer obtained by chemical grafting of phenyl diazoniums or azides.
16. The drug eluting stent of embodiments 14 and 15, wherein the grafted layer has a thickness between 10 nm and 1000 nm, preferably between 100 nm and 200 nm.
17. The drug eluting stent of embodiment 14, wherein the electro-grafted polymeric layer comprises a monomer selected from the group consisting of vinylics, epoxides, and cyclic monomers undergoing ring opening polymerization and aryl diazonium salts.
18. The drug eluting stent of embodiment 17, wherein the monomer is further selected from the group consisting of butyl methacrylate, methyl methacrylate, hydroxyethyl methacrylate, epsilon caprolactone, N-[Tris(hydroxymethyl)-methyl]-acrylamide (NTMA) and 4-nitrophenyl diazonium tetrafluoro borate.
19. A method of (i) selecting the product parameters of a drug eluting stent and/or (ii) predicting the outcome of the stent implantation at 1-year or more post-stent implantation (e.g., thrombosis), comprising preparing the stent and measuring the percentage of neointima coverage over the stent in the arterial tissue where a stent is implanted at 30 days post-stent implantation, wherein the higher the percentage of neointima coverage over the stent at 30 days, the better the stent in terms of stent efficacy and/or safety.
20. The method of embodiment 19, wherein the percentage of neointima coverage over the implanted stent at about 30 days/1 month is predictive of stent implantation side effects at 1-year or more post-stent implantation, wherein a 80-90% neointima coverage at about 30 days/1 month is representative or predictive of low side effects at 1-year post-stent implantation.
21. The method of embodiment 20, wherein the percentage of neointima coverage may be assessed by measuring strut coverage, preferably at about 30 days/1 month.
22. The method of embodiment 20, wherein the presence of neointima coverage may be assessed by OCT, preferably at about 30 days/1 month.
23. The method of embodiment 21, wherein a covered strut is a strut having a neointimal thickness above 0, preferably above 20, micrometers above the surface of the strut.
24. A method of preparing a drug-eluting stent, wherein the drug-eluting stent achieves between 80% and 100% neointima strut coverage between day 28 and day 90 post-stent implantation in an animal model, and preferably a rabbit iliac artery model, comprising preparing a stent with the properties of the stent of embodiment 1.
25. The method of embodiment 24, wherein 80%-100% neointima strut coverage is achieved between day 20 and day 60 post-stent implantation.
26. The method of embodiment 25, wherein 80%-100% neointima strut coverage is achieved about 30 days post-stent implantation.
27. A drug-eluting stent, comprising at least four parts:
   a stent framework;
   a drug-containing layer;
   a drug embedded in the drug-containing layer; and
   a biocompatible base layer disposed over the stent framework and supporting the drug-containing layer, wherein the stent has the following characteristics in a rabbit trial, after implantation in the iliac artery:
   (a) the uptake of Evans' Blue dye by the artery in the stented zone is <40% at 45 days and <25% at 90 days;
   (b) The ratio R, measured by confocal microscopy in a longitudinal cross-section of the stented zone of the stented artery, of the quantity of P120 protein to that of VE-Cadherin (R=[P120]/[VE-cad]), which characterizes the degree of co-localization of the said proteins in the scaffolded region, is higher than 70% at 45 days, and higher than 80% at 90 days; and
   (c) The cell shape index I, defined as the ratio between the largest length [a] of endothelial cells observed by confocal microscopy divided by the size [b] in the direction perpendicular to said longest length (I=[a]/[b]), is larger than 2 at 45 days after implantation, and larger than 3.5 at 90 days after implantation.
28. The stent of embodiment 27, wherein one or more parts of the stent are designed to achieve a pre-designed drug release pharmacokinetic profile selected from:
   (1) the drug pharmacokinetic profile having Tmax and Cmax (expressed in ng of drug per g of the artery tissue after implantation) so that:
      (a) Tmax is between 400 and 600 hr, preferably 500 hr,
      (b) Cmax is between 5 and 15 ng/g, preferably 10 ng/g, and/or
      (c) the drug pharmacokinetic profile overlaps with the kinetic profile for smooth muscle cell proliferation post-stent implantation, as depicted in FIG. 16B, preferably wherein the arterial tissue concentration at the site of stent implantation peaks between 15 and 25 days, preferably at 20 days, and then decreases to allow for vascular restoration; and
   (2) the drug has a pharmacokinetic profile in the arterial tissue at the site of stent implantation about as depicted in FIG. 16A or FIG. 16B, optionally, wherein the drug is sirolimus.
29. A method of preparing a drug-eluting stent, wherein the drug-eluting stent achieves between 80% and 100% neointima strut coverage between day 20 and day 60 post-stent implantation comprising preparing a stent with the properties of the stent of embodiment 27.
30. The method of embodiment 29, wherein 80%-100% neointima strut coverage is achieved between day 20 and day 60 post-stent implantation.
31. The method of embodiment 29, wherein 80%-100% neointima strut coverage is achieved about 30 days post-stent implantation.

Additional exemplary embodiments of this disclosure are provided below and numbered for reference purposes only:
1. A drug eluting stent, comprising:
   a stent framework;
   a drug-containing layer;
   a drug embedded in the drug-containing layer; and
   a biocompatible base layer disposed over the stent framework and supporting the drug-containing layer,
   wherein one or more parts of the stent are designed to achieve a pre-designed drug release pharmacokinetic profile selected from:
   (1) the drug pharmacokinetic profile has Tmax and Cmax (expressed in ng of drug per g of the artery tissue after implantation) so that:
      (a) Tmax is between 400 and 600 hr, preferably 500 hr,
      (b) Cmax is between 5 and 15 ng/g, preferably 10 ng/g, and/or
      (c) the drug pharmacokinetic profile overlaps with the kinetic profile for smooth muscle cell proliferation post-stent implantation, as depicted in FIG. 16B, preferably wherein the arterial tissue concentration at the site of stent implantation peaks between 15 and 25 days, preferably at 20 days, and then decreases to allow for vascular restoration; and
   (2) the drug has a pharmacokinetic profile in the arterial tissue at the site of stent implantation about as depicted in FIG. 16A or FIG. 16B.
2. The drug eluting stent of embodiment 1, wherein the drug-containing layer is configured to release the drug within 30 days of implantation within a vessel.
3. The drug eluting stent of embodiment 1, wherein a thickness of the drug-containing layer on a luminal side of the stent and a thickness of the drug-containing layer on a lateral side of the stent is less than a thickness of the drug-containing layer on an abluminal side of the stent.
4. The drug eluting stent of embodiment 3, where a ratio between the thickness of the drug-containing layer on the luminal side and the thickness of the drug-containing layer on the abluminal side is between 2:3 and 1:7.
5. The drug eluting stent of embodiment 3 or 4, where a ratio between the thickness of the drug-containing layer on the lateral side and the thickness of the drug-containing layer on the abluminal side is between 2:3 and 1:7.
6. The drug eluting stent of any one of embodiments 1 through 5, wherein the drug is embedded only on the drug-containing layer on an abluminal side of the stent.
7. The drug eluting stent of any one of embodiments 1 through 6, wherein the stent framework is fabricated from a single piece of metal, wire, or tubing.
8. The drug eluting stent of embodiment 7, wherein the metal comprises at least one of stainless steel, nitinol, tantalum, cobalt-chromium MP35N or MP20N alloys, platinum, and titanium.
9. The drug eluting stent of any one of embodiments 1 through 7, wherein the stent framework is fabricated from a biodegradable material, like for example a metallic alloy made from magnesium, zinc or iron.
10. The drug eluting stent of any one of embodiments 1 through 9, wherein the drug comprises at least one of an antithrombogenic agent, an anticoagulant, an antiplatelet agent, an antineoplastic agent, an antiproliferative agent, an antibiotic, an anti-inflammatory agent, a gene therapy agent, a recombinant DNA product, a recombinant RNA product, a collagen, a collagen derivative, a protein analog, a saccharide, a saccharide derivative, an inhibitor of smooth muscle cell proliferation, a promoter of endothelial cell migration, proliferation, and/or survival, and combinations of the same.
11. The drug eluting stent of embodiment 10, wherein the drug comprises sirolimus and/or a derivative or analog.
12. The drug eluting stent of embodiment 1, wherein the drug-containing layer has a thickness between 5 and 12 μm.
13. The drug eluting stent of embodiment 1, wherein the drug-containing layer is selected from the group consisting of poly(hydroxyalkanoates) (PHAs), poly(ester amides) (PEAs), poly(hydroxyalkanoate-co-ester amides), polyacrylates, polymethacrylates, polycaprolactones, poly(ethylene glycol)(PEG), poly(propylene glycol)(PPG), poly(propylene oxide) (PPO), poly(propylene fumarate) (PPF), poly(D-lactide), poly(L-lactide), poly(D,L-lactide), poly(meso-lactide), poly(L-lactide-co-meso-lactide), poly(D-lactide-co-meso-lactide), poly(D,L-lactide-co-meso-lactide), poly(D,L-lactide-co-PEG), poly(D,L-lactide-co-trimethylene carbonate), poly(lactide-co-glycolide), poly(glycolic acid-co-trimethylene carbonate), poly(trimethylene carbonate), PHA-PEG, PBT-PEG (PolyActive®), PEG-PPO-PEG (Pluronic®), and PPF-co-PEG, polycaprolactones, polyglycerol sebacate, polycarbonates, biopolyesters, polyethylene oxide, polybutylene terephalate, polydioxanones, hybrids, composites, collagen matrices with grouth modulators, proteoglycans, glycosaminoglycans, vacuum formed small intestinal submucosa, fibers, chitin, dexran and mixtures thereof.
14. The drug eluting stent of embodiment 13, wherein the drug-containing layer is selected from tyrosine derived polycarbonates.
15. The drug eluting stent of embodiment 13, wherein the drug-containing layer is selected from poly(β-hydroxyalcanoate)s and derivatives thereof.
16. The drug eluting stent of embodiment 13, wherein the drug-containing layer comprises a polylactide-co-glycolide 50/50 (PLGA).
17. The drug eluting stent of embodiment 1, wherein the biocompatible base layer comprises at least one of poly n-butyl methacrylate, poly-methyl methacrylate, poly-acrylic acid, poly-N-[Tris(hydroxymethyl)-methyl]-acrylamide (poly-NTMA), PEDOT (poly(3,4-ethylenedioxythiophene)) PTFE, PVDF-HFP, poly(styrene-b-isobutylene-b-styrene), Parylene C, Poly-DopAmine (PDA), PVP, PEVA, SBS, PC, TiO2 or any material that has good biocompatibility (or combinations thereof).
18. The drug eluting stent of embodiment 1, wherein the biocompatible base layer comprises an electro-grafted layer.
19. The drug eluting stent of embodiment 1, wherein the biocompatible base layer comprises an electro-polymerized layer.
20. The drug eluting stent of embodiment 18, wherein the electro-grafted layer is a polymeric layer interdigitating with the drug-containing layer.
21. The drug eluting stent of embodiment 18, wherein the electro-grafted polymeric layer has a thickness between 10 nm and 1000 nm.
22. The drug eluting stent of embodiment 18, wherein the electro-grafted polymeric layer comprises a monomer selected from the group consisting of vinylics, epoxides, and cyclic monomers undergoing ring opening polymerization and aryl diazonium salts.
23. The drug eluting stent of embodiment 22, wherein the monomer is further selected from the group consisting of butyl methacrylate, methyl methacrylate, hydroxyethyl methacrylate, epsilon caprolactone, and 4-nitrophenyl diazonium tetrafluoro borate.
24. A drug eluting stent, comprising:
   a stent framework;
   a biodegradable drug-containing layer;
   a drug embedded in the drug-containing layer; and
   a biocompatible base layer disposed over the stent framework and supporting the drug-containing layer,
   wherein the drug-containing layer is configured to significantly dissolve between 45 days and 90 days after implantation of the drug eluting stent.
25. The drug eluting stent of embodiment 24, wherein the drug-containing layer is formed from a plurality of polymers.
26. The drug eluting stent of embodiment 24, wherein one or more polymers forming the drug-containing layer on a luminal side of the stent and the drug-containing layer on a lateral side of the stent degrade faster than one or more polymers forming the drug-containing layers on an abluminal side of the stent.
27. The drug eluting stent of embodiment 24, wherein the stent framework is fabricated from a single piece of metal, wire, or tubing.
28. The drug eluting stent of embodiment 27, wherein the metal comprises at least one of stainless steel, nitinol, tantalum, cobalt-chromium MP35N or MP20N alloys, platinum, and titanium.
29. The drug eluting stent of embodiment 27, wherein the stent framework is fabricated from a biodegradable material, like for example a metallic alloy made from magnesium, zinc or iron.
30. The drug eluting stent of embodiment 24, wherein the drug comprises at least one of an antithrombogenic agent, an anticoagulant, an antiplatelet agent, an antineoplastic agent, an antiproliferative agent, an antibiotic, an anti-inflammatory agent, a gene therapy agent, a recombinant DNA product, a recombinant RNA product, a collagen, a collagen derivative, a protein analog, a saccharide, a saccharide derivative, an inhibitor of smooth muscle cell proliferation, a promoter of endothelial cell migration, proliferation, and/or survival, and combinations of the same.
31. The drug eluting stent of embodiment 24, wherein the drug comprises sirolimus and/or a derivative or analog.
32. The drug eluting stent of embodiment 24, wherein the drug-containing layer has a thickness between 5 and 12 μm.
33. The drug eluting stent of embodiment 24, wherein the drug-containing layer is selected from the group consisting of poly(hydroxyalkanoates) (PHAs), poly(ester amides) (PEAs), poly(hydroxyalkanoate-co-ester amides), polyacrylates, polymethacrylates, polycaprolactones, poly(ethylene glycol)(PEG), poly(propylene glycol)(PPG), poly(propylene oxide) (PPO), poly(propylene fumarate) (PPF), poly(D-lactide), poly(L-lactide), poly(D,L-lactide), poly(meso-lactide), poly(L-lactide-co-meso-lactide), poly(D-lactide-co-meso-lactide), poly(D,L-lactide-co-meso-lactide), poly(D,L-lactide-co-PEG), poly(D,L-lactide-co-trimethylene carbonate), poly(lactide-co-glycolide), poly(glycolic acid-co-trimethylene carbonate), poly(trimethylene carbonate), PHA-PEG, PBT-PEG (PolyActive®), PEG-PPO-PEG (Pluronic®), and PPF-co-PEG, polycaprolactones, polyglycerol sebacate, polycarbonates, biopolyesters, polyethylene oxide, polybutylene terephalate, polydioxanones, hybrids, composites, collagen matrices with grouth modulators, proteoglycans, glycosaminoglycans, vacuum formed small intestinal submucosa, fibers, chitin, dexran and mixtures thereof.
34. The drug eluting stent of embodiment 24, wherein the drug-containing layer is selected from tyrosine derived polycarbonates.
35. The drug eluting stent of embodiment 24, wherein the drug-containing layer is selected from poly(β-hydroxyalcanoate)s and derivatives thereof.
36. The drug eluting stent of embodiment 24, wherein the drug-containing layer comprises a polylactide-co-glycolide 50/50 (PLGA).
37. The drug eluting stent of embodiment 24, wherein the biocompatible base layer comprises at least one of poly n-butyl methacrylate, poly-methyl methacrylate, poly-acrylic acid, poly-N-[Tris(hydroxymethyl)-methyl]-acrylamide (poly-NTMA), PEDOT (poly(3,4-ethylenedioxythiophene)) PTFE, PVDF-HFP, poly(styrene-b-isobutylene-b-styrene), Parylene C, Poly-DopAmine (PDA), PVP, PEVA, SBS, PC, TiO2 or any material that has good biocompatibility (or combinations thereof).
38. The drug eluting stent of embodiment 24, wherein the biocompatible base layer comprises an electro-grafted layer.
39. The drug eluting stent of embodiment 24, wherein the biocompatible base layer comprises an electro-polymerized layer.
40. The drug eluting stent of embodiment 38, wherein the electro-grafted layer is a polymeric layer interdigitating with the drug-containing layer.
41. The drug eluting stent of embodiment 38, wherein the electro-grafted layer has a thickness between 10 nm and 1000 nm.
42. The drug eluting stent of embodiment 38, wherein the electro-grafted polymeric layer comprises a monomer selected from the group consisting of vinylics, epoxides, and cyclic monomers undergoing ring opening polymerization and aryl diazonium salts.
43. The drug eluting stent of embodiment 42, wherein the monomer is further selected from the group consisting of butyl methacrylate, methyl methacrylate, hydroxyethyl methacrylate, epsilon caprolactone, N-[Tris(hydroxymethyl)-methyl]-acrylamide and 4-nitrophenyl diazonium tetrafluoro borate.
44. A method of using the stent according to any one of embodiments 1 through 43, the method comprising implanting the stent into a subject for the treatment of stenosis or to prevent restenosis, thrombosis, tumor growth, angioma or, obstruction of lacrimal gland.
45. The method of embodiment 44, wherein the stent is implanted into a vessel.
46. The method of embodiment 45, wherein the vessel is the left main coronary artery, circumflex artery, left anterior descending coronary artery, an iliac vessel, a carotid artery, or a neurovascular vessel.
47. A method of treatment, comprising: a step of delivering the stent according to any one of embodiments 1 through 43 into a lumen; a step of radially expanding the stent within the lumen; and a step of eluting a drug from a drug coating layer in the surface of the stent allowing the drug to act on the lumen and/or albumen surface.
48. A method of reducing, minimizing, or eliminating patient risks associated with the implantation of a stent by using any one of the stents according to any one of embodiments 1 through 43.
49. A method of predicting long term stent efficacy and patient safety after implantation of a drug eluting stent, the method comprising assessing the percentage of functional restoration of the endothelium coverage of the stent and/or blood vessel after stent implantation in an animal model, wherein about 80%-100% neointima coverage at about 30 days post-stent implantation is predictive of long-term stent efficacy and patient safety after stent implantation. For example, the assessment may include using an animal model to assess the percentage of the coverage, a thickness and permeability of the endothelial layer and a structure of the endothelial layer. The structure may include the type of tissue, for example, the tissue composition in terms of smooth muscle cells, matrix, and endothelial cells.
50. The method of embodiment 49, wherein long-term stent efficacy comprises absence of significant restenosis of the vessel at the area of stent implantation.
51. The method of embodiment 49, wherein patient safety comprises absence of thrombosis of the vessel within and after 1-year post-stent implantation, preferably the thrombosis may be absent at 5 years post-stent implantation.
52. The method of embodiment 49, wherein patient safety comprises significant absence of MACE within 1-year and after post-stent implantation. preferably, the absence of MACE may be at 5 years post-stent implantation.
53. Use of the stent according to any one of embodiments 1 to 43 in the manufacture of a medicament or a device for treating or preventing a vascular disease, preferably angiostenosis or to prevent restenosis, thrombosis, tumor growth, angioma obstruction of lacrimal gland or neuro vasculature disease.

### BRIEF DESCRIPTION OF THE FIGURES

The drawings depict only exemplary embodiments of the present disclosure and do not therefore limit its scope. They serve to add specificity and detail to some of the embodiments.

The patent or application file contains at least one drawing executed in color. Copies of this patent or patent application publication with color drawings will be provided by the Office upon request and payment of the necessary fee.

FIG. 1A depicts a vessel (100) prior to implantation of a stent. 101 represents “active” of functional endothelial cells (ECs). 103 represents smooth muscle cells (SMCs).

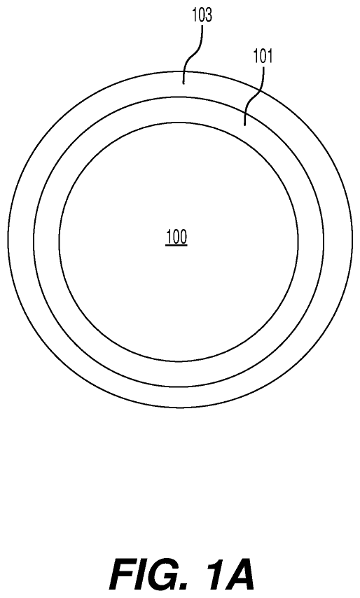

FIG. 1B depicts a vessel (150) after implantation of a stent. EC layer (101). Contact points (105a and 105b). SMC (103, 107a and 107b).

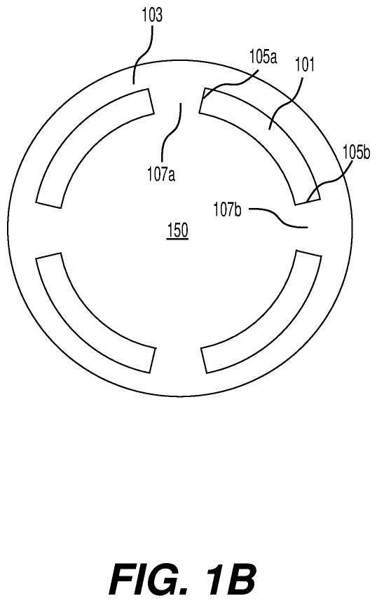

FIG. 2 depicts a Xience Xpedition stent 60 days after implantation in a rabbit iliac artery, imaged using SEM. The SEM images depict partial strut coverage with uncovered areas confined to middle and distal region of the stent. The percentage of endothelial coverage above stent struts is about 50%.

FIG. 3 depicts a drug eluting stent, according to some embodiments of the present disclosure, 60 days after implantation in a rabbit iliac artery, imaged using SEM. The SEM images depict a well-covered stent with few uncovered struts localized to the middle of the stent. The percentage of endothelial coverage above stent struts is about 80%.

FIG. 4A depicts a Xience Xpedition stent 60 days after implantation in a rabbit iliac artery, imaged using gross images with Evans Blue uptake, in which the positive stained area was 41.8%. (the presence of the staining is a negative marker for desirable endothelial cell layer functioning),

FIG. 4B depicts a confocal microscope image of the Xience Xpedition stent of FIG. 4A 60 days after implantation with tiling at lOx objective and with dual immunofluroescent staining of VE-Cadherin (red channel) and P120 (Endothelial p120-catenin) (green channel). The scale bar is 1 mm. The presence of good overlap (i.e. yellow) in staining is a positive marker of desirable endothelial cell layer functioning.

FIG. 4C depicts a confocal microscope image of a region of the Xience Xpedition stent of FIG. 4B 60 days after implantation with 20× objective, where the region had VE-Cadherin poorly expressed at endothelial borders, generally indicating poor barrier function. VE-Cadherin was red channel (555 nm), P120 was green channel (488 nm), and blue channel (405 nm) was DAPI (nuclei) counterstain. The scale bar is 50 μm. The presence of good overlap (i.e. yellow) in staining is a positive marker of desirable endothelial cell layer functioning

FIG. 4D depicts a confocal microscope image of another region of the Xience Xpedition stent of FIG. 4B with 20× objective, where the region had VE-Cadherin poorly expressed at endothelial borders, generally indicating poor barrier function. VE-Cadherin was red channel (555 nm), P120 was green channel (488 nm), and blue channel (405 nm) was DAPI counterstain. The scale bar is 50 μm. As depicted in FIGS. 4A-4D, endothelial coverage from both markers was 21.2% above the struts; and 21.2% between the struts=21.2%. The presence of good overlap (i.e. yellow) in staining is a positive marker of desirable endothelial cell layer functioning

FIG. 5A depicts a drug eluting stent, according to some embodiments of the present disclosure, 60 days after implantation imaged using gross images with Evans Blue uptake, in which the positive stained area was 35.7%. the presence of the staining is a negative marker for desirable endothelial cell layer functioning

FIG. 5B depicts a confocal microscope image of the drug eluting stent of FIG. 5A 60 days after implantation with tilting at 10× objective and with dual immunofluorescent staining of VE-Cadherin (red channel) and P120 (green channel). The scale bar is 1 mm. The presence of good overlap (i.e. yellow) in staining is a positive marker of desirable endothelial cell layer functioning

FIG. 5C depicts a confocal microscope image of a region of the drug eluting stent of FIG. 5B 45 days after implantation with 20× objective, where the region had partially endothelial barrier functioned area. VE-Cadherin was red channel (555 nm), P120 was green channel (488 nm), and blue channel (405 nm) was DAPI counterstain. The scale bar is 50 μm. The presence of good overlap (i.e. yellow) in staining is a positive marker of desirable endothelial cell layer functioning

FIG. 5D depicts a confocal microscope image of another region of the drug eluting stent of FIG. 5B 60 days after implantation with 20× objective, where the region had VE-Cadherin poorly expressed at endothelial borders, generally indicating poor barrier function. VE-Cadherin was red channel (555 nm), P120 was green channel (488 nm), and blue channel (405 nm) was DAPI counterstain. The scale bar is 50 μm. As depicted in FIGS. 5A-5D, endothelial coverage from both markers was 36.8% above the struts; and 38.8% between the struts. The presence of good overlap (i.e. yellow) in staining is a positive marker of desirable endothelial cell layer functioning

FIG. 6 depicts a Xience Xpedition stent 90 days after implantation imaged using SEM. The SEM images show partial stent coverage with uncovered areas mostly in the middle section. The percentage of endothelial coverage above stent struts is about 70%.

FIG. 7 depicts a drug eluting stent, according to some embodiments of the present disclosure, 90 days after implantation imaged using SEM. The SEM images show complete stent coverage. The percentage of endothelial coverage above stent struts is about 99%.

FIG. 8A depicts a Xience Xpedition stent 90 days after implantation using gross images with Evans Blue uptake, in which the positive stained area was 31.8% (the presence of the staining is a negative marker for desirable endothelial cell layer functioning).

FIG. 8B depicts a confocal microscope image of the Xience Xpedition stent of FIG. 8A 90 days after implantation with tiling at 10× objective and with dual immunofluroescent staining of VE-Cadherin (red channel) and P120 (green channel). The scale bar is 1 mm. The presence of good overlap (i.e. yellow) in staining is a positive marker of desirable endothelial cell layer functioning

FIG. 8C depicts a confocal microscope image of a region of the Xience Xpedition stent of FIG. 8B 90 days after implantation with 20× objective, where the region had evidence of competent endothelial barrier function (that is, co-localized p120/VE-cadherin). VE-Cadherin was red channel (555 nm), P120 was green channel (488 nm), and blue channel (405 nm) was DAPI counterstain. The scale bar is 50 μm. The presence of good overlap (i.e. yellow) in staining is a positive marker of desirable endothelial cell layer functioning

FIG. 8D depicts a confocal microscope image of another region of the Xience Xpedition stent of FIG. 8B 90 days after implantation with 20× objective, where the region had VE-Cadherin poorly expressed at endothelial borders, generally indicating poor barrier function. VE-Cadherin was red channel (555 nm), P120 was green channel (488 nm), and blue channel (405 nm) was DAPI counterstain. The scale bar is 50 μm. As depicted in FIGS. 8A-8D, endothelial coverage from both markers was 46.8% above the struts; and 46.1% between the struts. The presence of good overlap (i.e. yellow) in staining is a positive marker of desirable endothelial cell layer functioning

FIG. 9A depicts a drug eluting stent, according to some embodiments of the present disclosure, 90 days after implantation imaged using gross images with Evans Blue uptake, in which the positive stained area was 6.4%. the presence of the staining is a negative marker for desirable endothelial cell layer functioning

FIG. 9B depicts a confocal microscope image of the drug eluting stent of FIG. 9A 90 days after implantation with tilting at 10× objective and with dual immunofluorescent staining of VE-Cadherin (red channel) and P120 (green channel). The scale bar is 1 mm. The presence of good overlap (i.e. yellow) in staining is a positive marker of desirable endothelial cell layer functioning

FIG. 9C depicts a confocal microscope image of a region of the drug eluting stent of FIG. 9B 90 days after implantation with 20× objective, where the region had evidence of competent endothelial barrier function (that is, co-localized p120/VE-cadherin). VE-Cadherin was red channel (555 nm), P120 was green channel (488 nm), and blue channel (405 nm) was DAPI counterstain. The scale bar is 50 μm The presence of good overlap (i.e. yellow) in staining is a positive marker of desirable endothelial cell layer functioning.

FIG. 10 shows the drug release time frame of a XIENCE V stent and a XIENCE PRIME as about 120 days. The drug release time of ENDEAVOR RESOLUTE (i.e., a stent according to some embodiments of the present disclosure) as about 180 days.

FIG. 11 shows the relative position of layers of a stent according to some embodiments of the present disclosure. The luminal side (6) faces the blood flow, and the abluminal side (8) faces or contacts the vessel wall.

FIG. 12A depicts a drug eluting stent, according to some embodiments of the present disclosure, 45 days after implantation imaged using Evans Blue uptake, in which the positive stained area was 28.57%. (the presence of the staining is a negative marker for desirable endothelial cell layer functioning)

FIG. 12B depicts a drug eluting stent 45 days after implantation using Evans Blue uptake, in which the positive stained area was 55.0%. (the presence of the staining is a negative marker for desirable endothelial cell layer functioning)

FIG. 12C depicts a drug eluting stent 45 days after implantation imaged using Evans Blue uptake, in which the positive stained area was 56.79%. (the presence of the staining is a negative marker for desirable endothelial cell layer functioning)

FIG. 12D is a table summarizing the results of Evan's Blue update data at 45 day from experiments done with a stent according to embodiments of the present disclosure (BuMA Supreme) and not according to the present disclosure (Xience and Synergy).

FIG. 13A depicts a drug eluting stent, according to some embodiments of the present disclosure, 45 days after implantation showing a confocal microscope image of a region of the drug eluting stent with 20× objective, where the region had evidence of competent endothelial barrier function (that is, co-localized p120/VE-cadherin). VE Cadherin was red channel (555 nm), P120 was green channel (488 nm), and blue channel (405 nm) was DAPI counterstain. (The presence of the Evans blue staining is a negative marker for desirable endothelial cell layer functioning); The presence of good overlap (i.e. yellow) in staining is a positive marker of desirable endothelial cell layer functioning

FIG. 13B depicts a drug eluting stent 45 days after implantation showing a confocal microscope image of a region of the drug eluting stent with 20× objective, where the region had evidence of competent endothelial barrier function (that is, co-localized p120/VE-cadherin). VE Cadherin was red channel (555 nm), P120 was green channel (488 nm), and blue channel (405 nm) was DAPI counterstain. (The presence of the Evans blue staining is a negative marker for desirable endothelial cell layer functioning); The presence of good overlap (i.e. yellow) in staining is a positive marker of desirable endothelial cell layer functioning.

FIG. 13C depicts a drug eluting stent 45 days after implantation showing a confocal microscope image of a region of the drug eluting stent with 20× objective, where the region had evidence of competent endothelial barrier function (that is, co-localized p120/VE-cadherin). VE Cadherin was red channel (555 nm), P120 was green channel (488 nm), and blue channel (405 nm) was DAPI counterstain (blue/purple circles/nuclei). (The presence of the Evans blue staining is a negative marker for desirable endothelial cell layer functioning); The presence of good overlap (i.e. yellow) in staining is a positive marker of desirable endothelial cell layer functioning.

FIG. 13D is a table summarizing the results of the VE-Cadherin/P120 colocalization data at 45 days from experiments done with a stent according to embodiments of the present disclosure (BuMA Supreme) and not according to the present disclosure (Xience and Synergy).

FIG. 14A depicts a drug eluting stent, according to some embodiments of the present disclosure, 90 days after implantation imaged using Evans Blue uptake, in which the positive stained area was 23.21%. (The presence of the blue staining is a negative marker for desirable endothelial cell layer functioning)

FIG. 14B depicts a drug eluting stent 90 days after implantation using Evans Blue uptake, in which the positive stained area was 42.95%.%. (The presence of the blue staining is a negative marker for desirable endothelial cell layer functioning)

FIG. 14C depicts a drug eluting stent 90 days after implantation imaged using Evans Blue uptake, in which the positive stained area was 41.79%.%. (The presence of the blue staining is a negative marker for desirable endothelial cell layer functioning)

FIG. 14D is a table summarizing the results of Evan's Blue update data at 90 days from experiments done with a stent according to embodiments of the present disclosure (BuMA Supreme) and not according to the present disclosure (Xience and Synergy).

FIG. 15A depicts a drug eluting stent, according to some embodiments of the present disclosure, 90 days after implantation showing a confocal microscope image of a region of the drug eluting stent with 20× objective, where the region had evidence of competent endothelial barrier function (that is, co-localized p120/VE-cadherin). VE Cadherin was red channel (555 nm), P120 was green channel (488 nm), and blue channel (405 nm) was DAPI counterstain (The presence of the Evans blue staining is a negative marker for desirable endothelial cell layer functioning); The presence of good overlap (i.e. yellow) in staining is a positive marker of desirable endothelial cell layer functioning).

FIG. 15B depicts a drug eluting stent 90 days after implantation showing a confocal microscope image of a region of the drug eluting stent with 20× objective, where the region had evidence of competent endothelial barrier function (that is, co-localized p120/VE-cadherin). VE Cadherin was red channel (555 nm), P120 was green channel (488 nm), and blue channel (405 nm) was DAPI counterstain (The presence of the Evans blue staining is a negative marker for desirable endothelial cell layer functioning); The presence of good overlap (i.e. yellow) in staining is a positive marker of desirable endothelial cell layer functioning).

FIG. 15C depicts a drug eluting stent 90 days after implantation showing a confocal microscope image of a region of the drug eluting stent with 20× objective, where the region had evidence of competent endothelial barrier function (that is, co-localized p120/VE-cadherin). VE Cadherin was red channel (555 nm), P120 was green channel (488 nm), and blue channel (405 nm) was DAPI counterstain (The presence of the Evans blue staining is a negative marker for desirable endothelial cell layer functioning); The presence of good overlap (i.e. yellow) in staining is a positive marker of desirable endothelial cell layer functioning).

FIG. 15D is a table summarizing the results of the VE-Cadherin/P120 colocalization data at 90 days from experiments done with a stent according to embodiments of the present disclosure (BuMA Supreme) and not according to the present disclosure (Xience and Synergy).

FIG. 16A BuMA has an optimized release kinetic for an arterial peak of sirolimus at 20 days and fast polymer degradation.

FIG. 16B Pharmacokinetics designed to suppress SMC without limiting functional healing.

FIG. 16C BuMA has an optimized pharmacokinetic for a peak in arterial concentrations at 20 days.

FIG. 17 Study device characteristics

FIG. 18A-18F Assessment of endothelial permeability by Evans blue dye staining. A-D: Representative EBD staining examples of BP-SES (A), DP-EES (B), BP-EES (C) and BMS (D) at 45- (upper) and 90-day (lower). Each stent was sectioned longitudinally. E-F: Summarized 45- (E) and 90-day (F) data of the % EBD staining. The values represent the median with 25th-75th percentile. * P<0.05 vs BP-SES, †P<0.05 vs DP-EES, ‡P<0.05 vs BP-EES (generalized estimating equation). Abbreviations: BMS=bare metal stent (Multi-Link Vision®), BP-EES=biodegradable polymer everolimus-eluting stent (Synergy®), BP-SES=biodegradable polymer sirolimus-eluting stent (BuMA Supreme®), CoCr=cobalt-chromium, DP-EES=durable polymer everolimus-eluting stent (Xience Xpedition®).

FIG. 19A-19N Assessment of endothelial barrier protein expression and cell morphology by confocal microscopy. A-D: Representative confocal microscopic findings of BP-SES (A), DP-EES (B), BPEES(C) and BMS (D) at 45- (upper) and 90-day (lower). Each stent was sectioned longitudinally. 2 types of endothelial cell proteins, VE-cad (red) and p120 (green), were stained. E-F: Representative high-power image of area with co-localization of p120/VE-cad which shows spindle (E) and cobble-stone (F) endothelial cell shape. G: Representative high-power image of area without co-localization of p120/VE-cad. VE-cad appears within the cell cytoplasm but not at the cell-borders. H: Low-power image of representative border zone between complete and incomplete co-localization of p120/VE-cad at the area of white square in (C). The border is outlined in dotted red. I: Middle-power image of border zone between complete and incomplete co-localization of p120/VE-cad at the area of white square in (H). J: Schematic cartoon of endothelial cell shape index (height [a] divided by width [b]). K-N: Summarized data of % p120/VE-cad co-localization at stented segment (45- [K] and 90-day [L]), and average cell shape index (a/b ratio) (45- [M] and 90-day [N]) in each stent (BP-SES, DP-EES, BPEESand BMS). The values represent the median with 25th-75th percentile. * P<0.05 vs BP-SES, t P<0.05 vs DP-EES, $ P<0.05 vs BP-EES (generalized estimating equation.

FIG. 20A-20F Spatial distribution of Evans Blue staining and p120/VE-cadherin colocalization area. A: Example for co-registration of EBD and p120/VE-cad confocal images. EBD image (upper in A) and p120/VE-cad confocal image (lower in A) were co-registred via stent-struts using the confocal image of transmitted light detector channel (T-PMT) mode (mid in A) as an indicator. 6 side-by-side regions of interest (ROI) fields (400 μm×400 μm in each ROI) were randomly located in proximal, middle and distal stent parts of EBD image. Each field was co-registered with the location of stent-strut. In each ROI of EBD image, positive or negative staining was determined according to the following criteria (positive; >50% of the ROI field was stained by EBD, negative; <50% of the ROI field was stained). The same methodology was applied for p120/VE-cad confocal image (positive; >50% of the ROI field showed colocalization of p120/VE-cad, negative; <50% of the ROI field exhibited p120/VE-cad colocalization). B: High power fields for ROI 7-12 in A. According to the above mentioned criteria, these area were decided as follows (7; EBD[+]-p120/VE-cad[−], 8 and 10-12; EBD[−]-p120/VE-cad[+], 9; EBD[+]-p120/VE-cad[+]). Since EBD and p120/VE-cad confocal images were taken using different methods, completely exact matching could not achieved as shown in ROI 9 (one of the limitations of current assessment). C-F: Representative examples for EBD(+)-p120/VE-cad(+) (C and D) and EBD(−)-p120/VE-cad(+) (E and F) fields. EBD(+)-p120/VE-cad(+) area showed lower expression of VE-cad (red) at cellmembrane (D; box area in C) compared with EBD(−)-p120/VE-cad(+) area (F; box area in E). Abbreviations: ROI=region of interest.

FIG. 21 Comparison of Evans blue dye and p120/VE-cadherin for the detection of endothelial permeability.

FIG. 22A-22N Assessment of in-stent endothelialization by SEM, and co-registration with Evans blue dye staining and confocal microscopy. A-D: Representative SEM findings of BP-SES (A), DPEES(B), BP-EES (C) and BMS (D) at 45- (upper) and 90-day (lower). Each stent was sectioned longitudinally. E-G: Low-power image of representative border zone between complete and incomplete co-localization of p120 (green) with VE-cad (red) (E), co-registered area with EBD staining (F) and SEM (G), at the area surrounded by white square in (B). The border is outlined in dotted red. H-I: Middle-power image of border zone between complete and incomplete co-localization of p120/VE-cad (H), co-registered area with SEM (I), at the area surrounded by white square in (E) and (G), respectively. J-K: Representative high-power SEM images (lower) of the area which shows spindle (J) and cobble-stone (K) endothelial cell shape, co-registered with the area of p120/VE-cad co-localization in confocal image (upper). L: Representative high-power SEM image (lower) of the area which shows lack ofpl20/VE-cad co-localization in confocal image (upper). Adherent platelet and leukocyte aggregates at the site of intercellular ridge (black arrow). M-N: Summarized 45- (M) and 90-day (N) data of the % endothelial tissue coverage assessed with SEM in each stented segment (BP-SES, DP-EES, BP-EES and BMS). The values represent the median with 25th-75thpercentile. * P<0.05 vs BP-SES, †P<0.05 vs DP-EES, ‡P<0.05 vs BP-EES (generalized estimating equation). Abbreviations: SEM=scanning electron microscopy.

### DETAILED DESCRIPTION

The present disclosure relates to drug eluting stents, methods of making and using the drug eluting stents, as well as methods for predicting long term stent efficacy and patient safety after implantation of a drug-eluting stent. According to some embodiments of the present disclosure, the drug eluting stent (1) comprises four parts: a stent framework (2), a drug-containing layer (3), a drug (4), and a biocompatible base layer (5). In one embodiment, the stent may be made with stainless steel. In another embodiment, the stent may be made of CoCr alloy. In one embodiment, the stent has a thickness between 80-120 um. The drug-containing layer may be formed of PLGA, and the biocompatible base layer may be formed of PBMA. The biocompatible base layer may be formed using an electrografting process.

Discovery of Window of Time for Neointima Stent Coverage that Improves Vascular Restoration and Prevents Side Effects at a Later Time after Stent (1) Implantation

In one embodiment, the disclosure provides stents (1) where 80%-100% neointima coverage over the stent strut is achieved at an unexpected time period (preferably, 30 days) such that it prevents side effects from stent implantation later (e.g., 1 year and more), including restenosis and thrombosis. To this end, it was necessary to determine first at which time should neointima coverage over the stent strut be reached to prevent or reduce later (e.g., 1 year and more), side effects of stent implantation. In one embodiment, the disclosure provides that there is a window of opportunity for neointima coverage over the stent strut after the implantation of a DES stent into a vessel in terms of patient safety and stent efficacy. In one embodiment, 80-90% neointima coverage at an early time point (30 days) post-stent implantation results in improved endothelium/vascular restoration at a later time point (e.g., 1 year) that in turn minimizes side effects of stent implantation (.e.g., MACE). In one embodiment, endothelium/vascular restoration means that the proper connections among the endothelial cells are re-established, and the biological function of the endothelium is restored over the surface of the stent or along the vessel wall/neointima. In one embodiment, endothelium refers to a functional endothelial layer. In one embodiment, within the window time period disclosed herein (preferably, 30 days), 80%-100% neointima coverage of the stent strut can be obtained and restenosis and/or thrombosis be significantly prevented or reduced, and/or the duration of antiplatelet therapy may be abbreviated. In one embodiment, 80%-90%, preferably 80%-100%, neointima coverage of stent strut is obtained within the first 2-3 months, preferably first 30 days, such that the vascular endothelial function restoration can be achieved within 12 months. In one embodiment, 80%-100% neointima coverage occurs between 20 to 30 days, and between 80% and 95% of drug release occurs over the same period of time. In one embodiment, 80%-100% neointima coverage of stent strut is obtained within the first 20 days, or, most preferably within the first 30 days post stent implantation, and any time interval in between, such as, for example, between day 20 and day 30 of stent implantation. In one embodiment, 80%-100% neointima coverage of stent strut is obtained between day 30 and day 45 after stent implantation. In one embodiment, 80%-100% neointima coverage of stent strut is obtained within the first 30 days, 45, 60, 90, or at any day and any interval in between. In one embodiment, the stent shows 80% of neointima coverage at 30 days post-stent implantation, wherein the neointima is 20 μm thick. In one embodiment, the stent shows 90% of neointima coverage at 3 months post-stent implantation, wherein the neointima is more than 80 μm thick. In one embodiment, the stent shows 99% of neointima coverage at 12 months post-stent implantation, wherein the neointima is more than 150 μm thick. In one embodiment, the stent shows 80% of neointima coverage at 30 days post-stent implantation, wherein the neointima is 20 μm thick; 90% of neointima coverage at 3 months post-stent implantation, wherein the neointima is more than 80 μm thick; and 99% of neointima coverage at 12 months post-stent implantation, wherein the neointima is more than 150 μm thick. In one embodiment, the stent achieves functional restoration at 12 months. Neointima coverage of the stent struts can be assessed by any method known to one of ordinary skill in the art. In one embodiment, neointima coverage is assessed by OCT.

The sufficiency of the restoration of the endothelium can be determined by any means known in the industry. In animal models, this can be measured by methods that include SEM microscopy, Evans-blue staining (the presence of the staining is a negative marker for desirable endothelial cell layer functioning; e.g., at 30, 60, and 90 days; should not stain the endothelial layer), VE-Cadherin/p120 staining (the presence of good overlap in staining is a positive marker of desirable endothelial cell layer functioning), cell shape index, and others. See FIGS. for some examples, Animal data can be used to design the stent to meet these requirements which can be appropriately translated to stents for human use. In vivo, it may for example be measured by neointimal coverage of the surface of stent struts, and neointimal thickness as measured by Optical Coherence Tomography (OCT) and other methods known in art, at different time points. In one embodiment, neointimal thickness is measured at 1 month; 2 months; 3 months; 4 months; 5 months; 6 months, 7 months, 8 months, 9 months, and/or 12 months. In one embodiment, a thickness below a first threshold may be indicative that a sufficient foundation structure has not formed, which will result in less sufficient restoration of the function of the endothelial layer, while a thickness above a second, higher threshold may be indicative of a ratio of smooth muscle cells to endothelial cells that is too high, sometimes it is a good indication for over proliferation of the smooth muscle cells.

In one embodiment, a covered strut is defined as having a neointimal thickness above 20 micrometers (um). In some embodiments, the neointimal thickness is >20-120.0 um; e.g., 120.1-160.0 um. In a preferred embodiment, the neointimal thickness is between 20 and 160, preferably between 20 and 150 um at 2 months, in a rabbit iliac artery model. In some embodiments, the preferred neointima thickness in humans (measured by OCT) is between 20 and 80 um at 3 months, and preferably between 140 and 160 um at 12 months post-stent implantation. Stent coverage or neointimal coverage refers to global coverage of all struts, expressed in % of the global surface area of the whole stent.

In a preferred embodiment, the disclosure provides stents according to embodiment [087] in which the percentage of struts covered in human is higher than 80% at 1 month, and the percentage of uncovered struts is lower than 20%. And the neointima with a thickness between 20 and 80 um at one month, and preferably between 140 and 160 um at 12 months,

### Design of the Stent to Achieve Timely Vascular Restoration

Vascular restoration (or vascular healing) is defined as the re-establishment of the right connection among the endothelial cells so that the biological function of the endothelium is restored over the surface of the implanted stent or along the vessel wall/neointima. This functional restoration can be demonstrated or measured with several methods in the animal models, and in human. Those measurements include the coverage of the neointima over the stent strut at different time points; the thickness of the neointima at different time points; Evans-Blue staining; and immunological methods can be applied to characterize the functional of the endothelium as well. The level of the neointima coverage at early stage, preferably 30 days after the stent implantation is a good indicator for the level of complete vascular restoration at a later time point (e.g., 1 year). The higher level of neointima coverage at first 30 days after stent implantation will ensure less MACE after the 30 days. In one embodiment, 80-90%, preferably 80-100%, neointimal coverage over the stent strut, is achieved by 25, 26, 27, 28, 29, or 30 days post-stent implantation. In one embodiment, 80-90%, preferably 80-100%, neointimal coverage over the stent struts may be achieved by (or in the period of time between) 20-25 days, 26-30 days, 31-35 days, 36-40 days, 41-45 days, 46-50 days, 51-60 days/2 months post-stent implantation. In one embodiment, 80-90%, preferably 80-100%, neointimal coverage over the stent strut is obtained at day 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60/2 months. In one embodiment, preferably, max coverage of the stent, which is a key to ensure the complete vascular restoration, is achieved by 30 days/1 month post-stent implantation. All these values may be modified by the term “about.”

The percentage of neointimal coverage over the stent strut by any one of these days may be at least 80%, or at least 90%, or at least 91, 92, 93, 94, 95, 96, 97, 98, 99%. In one embodiment, 80-100% neointimal coverage over the stent strut is achieved, preferably, by 30 days/1 month. All these values may be modified by the term “about.”

In a preferred embodiment, 80-100% neointimal coverage over the stent strut is achieved by two months or at any period of time between day 30 and 2 months post-stent implantation. All these values may be modified by the term “about.”

To achieve this timing (80-90%, preferably 80-100%, neointima coverage, preferably by 30 days/1 month), many aspects of the stent may be manipulated or designed individually or in combination, including the stent framework (2), drug-containing layer (3), drug (4), and/or biocompatible base layer. In one embodiment, 80-100% neointimal coverage over the stent strut by, preferably, 30 days/1 month (or between day 30 and day 60, or by 2 months), is achieved by complete release of the drug and complete dissolution of the drug-containing layer, which can each alone or in combination be designed to be achieved at the following times:
(a) 25 days or less, and 30 days/1 month or less (or between day 30 and day 60, or by 2 months);
(b) between 25 days and 30 days/1 month, post-stent implantation, (or between day 30 and day 60, or by 2 months), and on any day or interval in between, post-stent implantation;
(c) After 15 but less than 25 days; 15 but less than 26 days; after 15 but less than 27 days; after 15 but less than 28 days; after 15 but less than 29 days; or after 15 but 30 days/1 month or less post-stent implantation (or between day 30 and day 60, or by 2 months);
(d) At day 25, 26, 27, 28, 29, or 30, or at 1 month (or between day 30 and day 60, or by 2 months);
(e) or the drug concentration in the arterial tissue area of the stent is reduced to zero, or about zero, 7 to 30 days after the peak of the SMC proliferation, preferably 7 to 14 days after the peak of the SMC proliferation.
(f) All these values may be modified by the term “about.”

In other embodiments, 80-90%, preferably 80-100%, neointima coverage over the stent strut is achieved by 30 days (or between day 30 and day 60, or by 2 months) through at least 80%, at least 90%, or at least 91, 92, 93, 94, 95, 96, 97, 98, 99% release of the drug and/or complete dissolution of the drug-containing layer.

In other embodiments, 80-90%, preferably 80-100%, neointima coverage over the stent strut is achieved by 30 days (or between day 30 and day 60, or by 2 months) through between 81-85-86-90-91-95-96-99% release of the drug and/or complete dissolution of the drug-containing layer.

In other embodiments, 80-90%, preferably 80-100%, neointimal coverage over the stent strut is achieved by 30 days (or between day 30 and day 60, or by 2 months) through between 81-90, 91-99% release of the drug and/or complete dissolution of the drug-containing layer.

In other embodiments, 80-90%, preferably 80-100%, neointimal coverage over the stent strut is achieved by 35 days (or between day 30 and day 60, or by 2 months) through at least 80%, at least 90%, or at least 91, 92, 93, 94, 95, 96, 97, 98, 99% release of the drug and/or complete dissolution of the drug-containing layer.

In other embodiments, 80-90%, preferably 80-100%, neointimal coverage over the stent strut is achieved by 35 days (or between day 30 and day 60, or by 2 months) through between 81-85-86-90-91-95-96-99% release of the drug and/or complete dissolution of the drug-containing layer.

In other embodiments, 80-90%, preferably 80-100%, neointimal coverage over the stent strut is achieved by 35 days (or between day 30 and day 60, or by 2 months) through between 81-90, 91-99% release of the drug and/or complete dissolution of the drug-containing layer.

In other embodiments, 80-90%, preferably 80-100%, neointimal coverage over the stent strut is achieved by 40 days (or between day 30 and day 60, or by 2 months) through at least 80%, at least 90%, or at least 91, 92, 93, 94, 95, 96, 97, 98, 99% release of the drug and/or complete dissolution of the drug-containing layer.

In other embodiments, 80-90%, preferably 80-100%, neointimal coverage over the stent strut is achieved by 40 days (or between day 30 and day 60, or by 2 months) through between 81-85-86-90-91-95-96-99% release of the drug and/or complete dissolution of the drug-containing layer.

In other embodiments, 80-90%, preferably 80-100%, neointimal coverage over the stent strut is achieved by 40 days (or between day 30 and day 60, or by 2 months) through between 81-90, 91-99% release of the drug and/or complete dissolution of the drug-containing layer.

In other embodiments, 80-90%, preferably 80-100%, neointimal coverage over the stent strut is achieved by 45 days (or between day 30 and day 60, or by 2 months) through at least 80%, at least 90%, or at least 91, 92, 93, 94, 95, 96, 97, 98, 99% release of the drug and/or complete dissolution of the drug-containing layer.

In other embodiments, 80-90%, preferably 80-100%, neointimal coverage over the stent strut is achieved by 45 days (or between day 30 and day 60, or by 2 months) through between 81-85-86-90-91-95-96-99% release of the drug and/or complete dissolution of the drug-containing layer.

In other embodiments, 80-90%, preferably 80-100%, neointimal coverage over the stent strut is achieved by 45 days (or between day 30 and day 60, or by 2 months) through between 81-90, 91-99% release of the drug and/or complete dissolution of the drug-containing layer.

In other embodiments, 80-90%, preferably 80-100%, neointimal coverage over the stent strut is achieved by 50 days (or between day 30 and day 60, or by 2 months) through at least at least 80%, at least 90%, or at least 91, 92, 93, 94, 95, 96, 97, 98, 99% release of the drug and/or complete dissolution of the drug-containing layer.

In other embodiments, 80-90%, preferably 80-100%, neointimal coverage over the stent strut is achieved by 50 days (or between day 30 and day 60, or by 2 months) through between 81-85; 86-90; 91-95; 96-99% release of the drug and/or complete dissolution of the drug-containing layer.

In other embodiments, 80-90%, preferably 80-100%, neointimal coverage over the stent strut is achieved by 50 days (or between day 30 and day 60, or by 2 months) through between 81-90, 91-99% release of the drug and/or complete dissolution of the drug-containing layer.

In other embodiments, 80-90%, preferably 80-100%, neointimal coverage over the stent strut is achieved by 55 days (or between day 30 and day 60, or by 2 months) through at least at least 80%, at least 90%, or at least 91, 92, 93, 94, 95, 96, 97, 98, 99% release of the drug and/or complete dissolution of the drug-containing layer.

In other embodiments, 80-90%, preferably 80-100%, neointimal coverage over the stent strut is achieved by 55 days (or between day 30 and day 60, or by 2 months) through between 81-85-86-90-91-95-96-99% release of the drug and/or complete dissolution of the drug-containing layer.

In other embodiments, 80-90%, preferably 80-100%, neointimal coverage over the stent strut is achieved by 55 days (or between day 30 and day 60, or by 2 months) through between 81-90, 91-99% release of the drug and/or complete dissolution of the drug-containing layer.

In other embodiments, 80-90%, preferably 80-100%, neointimal coverage over the stent strut is achieved by 60 days/2 (or between day 30 and day 60, or by 2 months) months through at least at least 80%, at least 90%, or at least 91, 92, 93, 94, 95, 96, 97, 98, 99% release of the drug and/or complete dissolution of the drug-containing layer.

In other embodiments, 80-90%, preferably 80-100%, neointimal coverage over the stent strut is achieved by 60 days/2 months (or between day 30 and day 60, or by 2 months) through between 81-85; 86-90; 91-95; 96-99% release of the drug and/or complete dissolution of the drug-containing layer.

In other embodiments, 80-90%, preferably 80-100%, neointimal coverage over the stent strut is achieved by 60 days/2 months (or between day 30 and day 60, or by 2 months) through between 81-90, 91-99% release of the drug and/or complete dissolution of the drug-containing layer.

All of the above days and percentages may be modified by the term “about.”

The following represent some of the means for achieving these timings, level of drug release, and level of drug-containing layer dissolution. These are not limiting embodiments of the disclosure.

### (2). Stent Framework

Stents (1) are typically composed of a scaffold or scaffolding that includes a pattern or network of interconnecting structural elements or struts, formed from wires, tubes, or sheets of material rolled into a cylindrical shape. This scaffold gets its name because it physically holds open and, if desired, expands the wall of a passageway in a patient. Typically, stents are capable of being compressed or crimped onto a catheter so that they can be delivered to and deployed at a treatment site. Delivery includes inserting the stent through small lumens using a catheter and transporting it to the treatment site. Deployment includes expanding the stent to a larger diameter once it is at the desired location.

A stent framework (2) may be fabricated from a single (or more) piece(s) of metal or wire or tubing, including the 3D printing and laser cutting (e.g., starting from a wire). For example, the stent framework may be non-stainless steel or comprise stainless steel, nitinol, tantalum, cobalt-chromium (e.g., MP35N or MP20N alloys), platinum, titanium, suitable biocompatible alloys, other suitable biocompatible materials, and/or combinations thereof. In some embodiments, the stent is a non-stainless steel stent. In other embodiments, the stent framework may be fabricated from a metallic material or an alloy such as, but not limited to, cobalt chromium alloy (ELGILOY), stainless steel (316L), high nitrogen stainless steel, e.g., BIODUR 108, cobalt chrome alloy L-605, nitinol (nickel-titanium alloy), tantalum, platinum-iridium alloy, gold, magnesium, zinc, iron, or combinations thereof. “MP35N” and “MP20N” are trade names for alloys of cobalt, nickel, chromium and molybdenum available from Standard Press Steel Co., Jenkintown, Pa. “MP35N” consists of 35% cobalt, 35% nickel, 20% chromium, and 10% molybdenum. “MP20N” consists of 50% cobalt, 20% nickel, 20% chromium, and 10% molybdenum. In other embodiments, the stent framework (2) may be fabricated from one or more plastics, for example, polyurethane, teflon, polyethylene, or the like. In such embodiments, the stent framework (2) may be fabricated, for example, using 3-D printing.

The stent framework (2) may form a mesh. Accordingly, the stent framework (2) may expand upon implantation, either from external forces such as from a balloon catheter and/or from internal forces such as expansion of the mesh caused by increased temperature within the vessel. Upon expansion, the stent framework (2) may hold the vessel open.

In some embodiments, the stent framework (2) may be biodegradable. In order to effect healing of a diseased blood vessel, the presence of the stent is necessary only for a limited period of time, as the artery undergoes physiological remodeling over time after deployment. The development of a bioabsorbable stent or scaffold could obviate the permanent metal implant in the vessel, allow late expansive luminal and vessel remodeling, and leave only healed native vessel tissue after the full resorption of the scaffold. Stents fabricated from bioresorbable, biodegradable, bioabsorbable, and/or bioerodable materials such as bioabsorbable polymers can be designed to completely absorb only after or some time after the clinical need for them has ended. Consequently, a fully bioabsorbable stent can reduce or eliminate the risk of potential long-term complications and of late thrombosis, facilitate non-invasive diagnostic MRI/CT imaging, allow restoration of normal vasomotion, and provide the potential for plaque regression. For example, the sent framework (2) may be fabricated from chitosan, magnesium alloy, polylactic acid, polycarbonate polymers, salicylic acid polymers, and/or combinations thereof. Advantageously, a biodegradable stent framework (2) may allow for the vessel to return to normalcy after a blockage has been cleared and flow restored by the stent (1). The term “biodegradable” as used herein is interchangeable with the terms “bioabsorbable” or “bioerodable”, and generally refers to polymers or certain specific alloys, such as magnesium alloy, that are capable of being completely degraded and/or eroded when exposed to bodily fluids such as blood and can be gradually resorbed, absorbed, and/or eliminated by the body. The processes of breaking down and absorption of the polymer in a stent can be caused by, for example, hydrolysis and metabolic processes.

“A biodegradable stent” is used herein to mean a stent made from biodegradable polymers. Additional representative examples of polymers that may be used for making a biodegradable stent include, but are not limited to, poly(N-acetylglucosamine) (chitin), chitosan, poly(hydroxyvalerate), poly(lactide-coglycolide), poly(hydroxybutyrate), poly(hydloxybutyrate co-valerate), polyorthoester, polyanhydride, poly(glycolic acid), poly(glycolide), poly(L-lactic acid), poly(L-lactide), poly(D,L-lactic acid), poly(D,L-lactide), poly(caprolactone), poly(trimethylene carbonate), polyester amide, poly(glycolic acid-co-trimethylene carbonate), co-poly(ether-esters) (e.g. PEO/PLA), polyphosphazenes, biomolecules (such as fibrin, fibrinogen, cellulose, starch, collagen and hyaluronic acid). Another type of polymer based on poly(lactic acid) that can be used includes graft copolymers, and block copolymers, such as AB block-copolymers (“diblock-copolymers”) or ABA block-copolymers (“triblock-copolymers”), or mixtures thereof.

Additional representative examples of polymers that may be suited for use in fabricating a biodegradable stent include ethylene vinyl alcohol copolymer (commonly known by the generic name EVOI-I or by the trade name EVAL. The properties and usages of these biodegradable polymers are known in the art, for example, as disclosed in U.S. Pat. No. 8,017,144 and U.S. application publication No. 2011/0,098,803.

In some aspects, a biodegradable stent as described herein may be made from polylactic acid (PLA), polyglycolic acid (PGA), poly(D,L-lactide-co-glycolide), polycaprolactone, or copolymers thereof. In some aspects, a biodegradable stent as described herein may be made from polyhydroxy acids, polyalkanoates, polyanhydrides, polyphosphazenes, polyetheresters, polyesteramides, polyesters, and polyorthoesters. In some preferable aspects, a biodegradable stent as described herein may be made from chitosan, collagen, elastin, gelatin, fibrin glue, or combinations thereof.

“Chitosan based stent”, “chitosan stent” as described herein mean that the major component of a stent comes from chitosan. For example, a chitosan based stent as described herein may contain chitosan at least in an amount of over 50%, or over 60%, or over 70%, or over 80% weight percentage of the total stent weight. Even more particularly, a chitosan based stent as described herein may have the chitosan content in an amount of between about 70% and about 85% weight percentage of the total chitosan stent.

A chitosan-based stent as described herein may also be coated with a polymer layer in order to adjust degradation times. For example, a chitosan based stent as described herein may be dip-coated with a solution of poly(D,L-lactide-co-glycolide) in acetone. A chitosan-based stent may also be coated with a layer of barium sulfate, by dipping the stents into an aqueous suspension of barium sulfate. in some aspects, the weight of the coated barium sulfate may be in an amount of between about 15 and between about 30 weight percentage of the total weight of the stent. Additionally, a chitosan stent may be perforated.

The stent designed according to the criteria of this disclosure may be a coronary stent, a vascular stent, or any other drug-containing implantable devices for vascular system as well any medical device that is effective in lowering the restenosis and thrombosis rates in a sustainable manner to secure patient safety in the long term.

In one embodiment, a thinner stent is used. However, the stent strut should have enough thickness which will ensure the stent structure stability, without the risk of breaking over time. As an example, the thickness of the stent for 316L stainless steel stent is about 100 to 1l0 um, and for the CoCr stent is about 80 μm. In one embodiment, the stent thickness is between 100 and less than 120 μm. In one embodiment, the stain thickness is between 80 μm and 120 μm. In one embodiment, the stent thickness is between 5-10, 11-15, 16-20, 21-25, 26-30, 31-35, 36-40, 41-45, 46-50, 51-55, 56-60, 61-65, 66-70, 71-75, 75-80, 81-85, 86-90, 91-95-96-99, 100-105, 106-110, 111-15, 116-120, or between 60 and 120 μm, and any thickness and thickness interval in between. All these values may be modified by the term “about.”

### (3). Drug-Containing Layer

In one embodiment, the drug eluting stent, is designed in such way that it can achieve complete drug release within 30 days, and substantial neointimal coverage at 3 months. Substantial neointimal coverage and how to measure it is described above. In one embodiment, the drug eluting stent, is designed in such way that the drug-containing layer can achieve complete drug release within 30 days, and substantial neointimal coverage at 30 days, 40 days, 50 days etc, less than 100 days, preferably within more than 21 and 30 days or less. In one embodiment, the drug eluting stent is designed in such way that the drug-containing layer completely dissolves within a certain interval (20-30; 31-40, 41-50, etc and/or less than 100 days after stent implantation. Examples of how to make a drug-containing layer according to some embodiments of this disclosure are provided in the EXAMPLES section further below.

For the purposes of this disclosure, “complete drug release” from the stent (drug-containing layer) means release of from about 80% to about 100% of the drug, preferably from about 95%- to about 96%, from about 96%- to about 97%, from about 97%- to about 98%, from about 98% to about 99%, and from about 99%- to about 100% of the drug. Drug release is assessed in animal models (e.g., rabbit model) or in vitro models that are understood by one of ordinary skill in the art as predictable of drug release in the subject in which the stent of the disclosure is implanted. In one embodiment, “completely released” refers to a level at which the drug remaining is below detectable level and/or below a therapeutic level.

For the purposes of this disclosure, the drug-containing layer is said to have “completely dissolved” (also referred to as bio-degraded) when from about 95% to about 100% of the drug-containing layer, preferably from about 95%- to about 96%, from about 96%- to about 97%, from about 97%- to about 98%, from about 98% to about 99%, and from about 99%- to about 100% of the drug-containing layer has dissolved (also referred to as bio-degraded) from the stent. Drug-containing layer dissolution (also referred to as bio-degradation) from the stent is assessed in animal models (e.g., rabbit model) or in vitro models that are understood by one of ordinary skill in the art as predictable of the drug-containing layer dissolution (also referred to as bio-degradation) from the stent in the subject in which the stent of the disclosure is implanted. In one embodiment, “completely dissolved” refers to a level at which the material remaining is below a detectable level.

A drug-containing layer (3) may be made from polymers and may comprise a layer or layers covering all or part of the stent surface. Furthermore, a drug-containing layer (3) may be capable of hosting a drug (4) and releasing the drug (4) in a sustained manner. Examples of the polymers using in drug-containing layer (3) may include, but are not limited to, poly(hydroxyalkanoates) (PHAs), poly(ester amides) (PEAs), poly(hydroxyalkanoate-co-ester amides), polyacrylates, polymethacrylates, polycaprolactones, poly(ethylene glycol)(PEG), poly(propylene glycol)(PPG), poly(propylene oxide) (PPO), poly(propylene fumarate) (PPF), poly(D-lactide), poly(L-lactide), poly(D,L-lactide), poly(meso-lactide), poly(L-lactide-co-meso-lactide), poly(D-lactide-co-meso-lactide), poly(D,L-lactide-co-meso-lactide), poly(D,L-lactide-co-PEG), poly(D,L-lactide-co-trimethylene carbonate), poly(lactide-co-glycolide), poly(glycolic acid-co-trimethylene carbonate), poly(trimethylene carbonate), PHA-PEG, PBT-PEG (PolyActive®), PEG-PPO-PEG(Pluronic®), and PPF-co-PEG, polycaprolactones, polyglycerol sebacate, polycarbonates, biopolyesters, polyethylene oxide, polybutylene terephalate, polydioxanones, hybrids, composites, collagen matrices with growth modulators, proteoglycans, glycosaminoglycans, vacuum formed small intestinal submucosa, fibers, chitin, dexran, and/or mixtures thereof.

The rate of degradation of the drug-containing polymer layer is generally determined by its composition. One of ordinary skill in the art may select one or more polymers using a standard PK animal test to confirm that the polymer(s) degrade between 45 and 60 days after implantation. In addition, a manufacturer of the polymer or the polymeric matrix may provide the degradation performance of the drug-containing polymer, e.g., the degradation curve. One of ordinary skill in the art may derive the rate of degradation of the drug-containing polymer(s) from the degradation performance and select the polymer(s) based on the rate of degradation.

In one embodiment, the drug-containing layer (3) may have a thickness between 1 and 200 μm, e.g., between 5 and 12 μm. In one embodiment, the drug-containing layer has a thickness between 3.5-10 μm. In one embodiment, the thickness of the abluminal side is between 1.5-200 μm and the thickness of the luminal side is between 1-66 μm.

In certain aspects, the drug-containing layer (3) may have an uneven coating thickness. For example, the coating thickness of the luminal side (6) and the lateral side (7) may be thinner than the abluminal side (8) of the stent. In one embodiment, a coating thickness ratio between the luminal side (6) and the abluminal side (8) may range from 2:3 to 1:7. Similarly, the coating thickness ratio between the lateral side (7) and the abluminal side (8) may range from 2:3 to 1:7. Accordingly, the drug release on the luminal side (6) and the lateral side (7) may be faster than the abluminal side (8). The faster release of the drug on the luminal side (6) and the lateral side (7) may enable faster restoration of endothelia layers on the luminal side (6) and the lateral side (7) compared with the abluminal side (8). In another embodiment, the coating thickness ratio between the luminal side (6) and the abluminal side (8) may be 1:1. Ranges provided herein are understood to be shorthand for all of the values within the range. For example, a range of 1 to 10 is understood to include any number, combination of numbers, or sub-ranges such as 1, 1.5, 2.0, 2.8, 3.90, 4, 5, 6, 7, 8, 9, and 10.

In some embodiments, the drug-containing layer (3) may be coated on the abluminal side (8) of the stent only. In such embodiments, the lack of drug release from the luminal side (6) and the lateral side (7) may enable the early restoration of endothelia layers on the luminal side (6) and the lateral side (7). In other embodiments, the drug release from the luminal side (6) and the lateral side (7) may be less than 15 days, or 10-20 days, which may enable the early restoration of endothelial layers on the luminal side (6) and the lateral side (7).

Moreover, in such embodiments, the degradation of polymer on the luminal side (6) and the lateral side (7) may be faster than the degradation of polymer on the abluminal side (8). For example, the polymer of the luminal side (6) and the lateral side (7) may comprise PLGA, and the polymer of the abluminal side (8) may comprise PLA. Generally, the degradation of PLGA is faster than PLA, and this information can be easily accessed from the polymer manufacturer.

In some embodiments, sometimes advantageously, a 30-day drug (4) release time frame and a 45-to-60-day drug-containing coating (3) bio-degradable/dissolution time frame may enable the functional restoration of endothelial layers. Within the time frame mentioned above, the restoration of the functional EC layer may be sufficiently completed in 30 days, 45 days, 60 days, or 90 days (and any interval or data point in between) as measured in rabbit animal model. Then it may enable the long-term safety of the drug eluting stent in human. In one embodiment, the stent is unevenly coated by the drug containing layer, producing a thinner drug coating on the luminal or luminal side of the stent, which enables the drug to disappear from the stent between 10 to 20 days, 30 days (or more than 30 days), 40 days, 45 days, 60 days, etc or more, and less than 100 days.

The drug-containing coating may soften, dissolve or erode from the stent to elute at least one bioactive agent. This elution mechanism may be referred to as surface erosion where the outside surface of the drug-polymer coating dissolves, degrades, or is absorbed by the body; or bulk erosion where the bulk of the drug-polymer coating biodegrades to release the bioactive agent. Eroded portions of the drug-polymer coating may be absorbed by the body, metabolized, or otherwise expelled. In the case of bulk erosion, the drug-containing polymer layers tends to disappear non uniformly over the stent surface, so that some areas are free from the biodegradable polymer—enabling local contact between the biological medium and the biocompatible base layer—while some other areas are still covered by the degrading drug-containing layer.

The drug-containing coating may also include a polymeric matrix. For example, the polymeric matrix may include a caprolactone-based polymer or copolymer, or various cyclic polymers. The polymeric matrix may include various synthetic and non-synthetic or naturally occurring macromolecules and their derivatives. The polymer is advantageously selected in the group consisting of one or more biodegradable polymers in varying combinations, such as polymers, copolymers, and block polymers. Some examples of such biodegradable (also bio-resorbable or else bioabsorbable) polymers include polyglycolides, polylactides, polycaprolactones, polyglycerol sebacate, polycarbonates e.g. tyrosine derived, biopolyesters such as poly(β-hydroxyalcanoate)s (PHAs) and derived compounds, polyethylene oxide, polybutylene terepthalate, polydioxanones, hybrids, composites, collagen matrices with growth modulators, proteoglycans, glycosaminoglycans, vacuum formed SIS (small intestinal submucosa), fibers, chitin, and dextran. Any of these biodegradable polymers may be used alone or in combination with these or other biodegradable polymers in varying compositions. The polymeric matrix preferably includes biodegradable polymers such as polylactide (PLA), polyglycolic acid (PGA) polymer, poly (e-caprolactone) (PCL), polyacrylates, polymethacryates, or other copolymers. The pharmaceutical drug may be dispersed throughout the polymeric matrix. The pharmaceutical drug or the bioactive agent may diffuse out from the polymeric matrix to elute the bioactive agent. The pharmaceutical drug may diffuse out from the polymeric matrix and into the biomaterial surrounding the stent. The bioactive agent may separate from within the drug-polymer and diffuse out from the polymeric matrix into the surrounding biomaterial. In a further embodiment the drug coating composition may be fashioned using the drug 42-Epi-(tetrazolyl)-Sirolimus, set forth in U.S. Pat. No. 6,329,386 assigned to Abbott Laboratories, Abbott Park, Ill. and dispersed within a coating fashioned from phosphorylcholine coating of Biocompatibles International P.L.C. set forth in U.S. Pat. No. 5,648,442.

The polymeric matrix of the drug-containing layer may be selected to provide a desired elution rate of the drug/bioactive agent. The pharmaceutical drugs may be synthesized such that a particular bioactive agent may have two different elution rates. A bioactive agent with two different elution rates, for example, would allow rapid delivery of the pharmacologically active drug within twenty-four hours of surgery, with a slower, steady delivery of the drug, for example, over the next two to six months. The electro-grafted primer coating may be selected to firmly secure the polymeric matrix to the stent framework, the polymeric matrix containing the rapidly deployed bioactive agents and the slowly eluting pharmaceutical drugs.

### (4) The Drug or Bioactive Agent

In some embodiments, a drug (4) may be encapsulated/dissolved in the drug-containing layer (3) using a microbead, microparticle or nanoencapsulation technology with albumin, liposome, ferritin or other biodegradable proteins and phospholipids, prior to application on the primer-coated stent. By way of example, drug (4) may include, one or more of for example, an antithrombogenic agent, an anticoagulant, an antiplatelet agent, an antineoplastic agent, an antiproliferative agent, an antibiotic, an anti-inflammatory agent, a gene therapy agent, a recombinant DNA product, a recombinant RNA product, a collagen, a collagen derivative, a protein analog, a saccharide, a saccharide derivative, an inhibitor of smooth muscle cell proliferation, a promoter of endothelial cell migration, proliferation, and/or survival, and combinations of the same. In one embodiment, the drug is an anti-angiogenic drug. In another embodiment, the drug is an angiogenic drug. In some embodiments, the drug/bioactive agent may control cellular proliferation. The control of cell proliferation may include enhancing or inhibiting the growth of targeted cells or cell types. In some embodiments, the cells are vascular smooth muscle cells, endothelial cells, or both. In some embodiments, the drug suppresses the proliferation of smooth muscle cells and/or promotes the proliferation of endothelial cells.

More broadly, the drug (4) may be any therapeutic substance that provides a therapeutic characteristic for the prevention and treatment of disease or disorders whereby the use of a stent of the disclosure is appropriate. For example, an antineoplastic agent may prevent, kill, or block the growth and spread of cancer cells in the vicinity of the stent. In another example, an antiproliferative agent may prevent or stop cells from growing. In yet a further example, an antisense agent may work at the genetic level to interrupt the process by which disease-causing proteins are produced. In a fourth example, an antiplatelet agent may act on blood platelets, inhibiting their function in blood coagulation. In a fifth example, an antithrombogenic agent may actively retard blood clot formation. According to a sixth example, an anticoagulant may delay or prevent blood coagulation with anticoagulant therapy, using compounds such as heparin and coumarins. In a seventh example, an antibiotic may kill or inhibit the growth of microorganisms and may be used to combat disease and infection. In an eighth example, an anti-inflammatory agent may be used to counteract or reduce inflammation in the vicinity of the stent. According to a ninth example, gene therapy agent may be capable of changing the expression of a person's genes to treat, cure or ultimately prevent disease. In addition, an organic drug may be any small-molecule therapeutic material, and, similarly, a pharmaceutical compound may be any compound that provides a therapeutic effect. A recombinant DNA product or a recombinant RNA product may include altered DNA or RNA genetic material. In another example, bioactive agents of pharmaceutical value may also include collagen and other proteins, saccharides, and their derivatives. For example, the bioactive agent may be selected to inhibit vascular restenosis, a condition corresponding to a narrowing or constriction of the diameter of the bodily lumen where the stent is placed

Alternatively or concurrently, the bioactive agent may be an agent against one or more conditions, including, but not limited to, coronary restenosis, cardiovascular restenosis, angiographic restenosis, arteriosclerosis, hyperplasia, and other diseases and conditions. For example, the bioactive agent may be selected to inhibit or prevent vascular restenosis, a condition corresponding to a narrowing or constriction of the diameter of the bodily lumen where the stent is placed. The bioactive agent may alternatively or concurrently control cellular proliferation. The control of cell proliferation may include enhancing or inhibiting the growth of targeted cells or cell types.

Examples of antiplatelets, anticoagulants, antifibrin, and antithrombins include sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, thrombin inhibitors such as Angiomax™ (bivalirudin, Biogen, Inc., Cambridge, Mass.), calcium channel blockers (such as nifedipine), colchicine, fibroblast growth factor (FGF) antagonists, fish oil (omega 3-fatty acid), histamine antagonists, lovastatin (an inhibitor of HMG-CoA reductase, a cholesterol lowering drug, brand name Mevacor® from Merck & Co., Inc., Whitehouse Station, N.J.), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), nitric oxide, nitric oxide donors, super oxide dismutases, super oxide dismutase mimetic, 4-amino-2,2,6,6-tetramethylpiperidine-1-oxyl (4-amino-TEMPO), estradiol, dietary supplements such as various vitamins, and combinations thereof.

In some embodiments, the bioactive agent may include podophyllotoxin, etoposide, camptothecin, a camptothecin analog, mitoxantrone, Sirolimus (rapamycin), everolimus, zotarolimus, Biolimus A9, myolimus, deforolimus, AP23572, tacrolimus, temsirolimus, pimecrolimus, novolimus, zotarolimus (ABT-578), 40-O-(2-hydroxy)ethyl-rapamycin (everolimus), 40-O-(3-hydroxypropyl)rapamycin, 40-O-[2-(2-hydroxy)ethoxy]ethyl-rapamycin, 40-O-tetrazolylrapamycin, 40-epi-(N1-tetrazolyl)-rapamycin, and their derivatives or analogs. Podophyllotoxin is generally an organic, highly toxic drug that has antitumor properties and may inhibit DNA synthesis. Etoposide is generally an antineoplastic that may be derived from a semi-synthetic form of podophyllotoxin to treat monocystic leukemia, lymphoma, small-cell lung cancer, and testicular cancer. Camptothecin is generally an anticancer drug that may function as a topoisomerase inhibitor. Related in structure to camptothecin, a camptothecin analog, such as aminocamptothecin, may also be used as an anticancer drug. Mitoxantrone is an anticancer drug generally used to treat leukemia, lymphoma, and breast cancer. Sirolimus is a medication that generally interferes with the normal cell growth cycle and may be used to reduce restenosis. The bioactive agent may alternatively or concurrently include analogs and derivatives of these agents. Antioxidants may be used in combination with or individually from the examples above for their antirestonetic properties and therapeutic effects.

The anti-inflammatory agent can be a steroidal anti-inflammatory agent, a nonsteroidal anti-inflammatory agent, or a combination thereof. In some embodiments, anti-inflammatory drugs include, but are not limited to, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, momiflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicylic acid), salicylic acid, corticosteroids, glucocorticoids, tacrolimus, pimecorlimus, prodrugs thereof, co-drugs thereof, and combinations thereof.

For the removal of blood clots and thrombus, examples of therapeutic agents may include (i) tissue plasminogen activator, tPA, BB-10153, rTPA, Urokinease, Streptokinase, Alteplase and Desmoteplase, (ii) antiplatelet agents such as aspirin, Clopidogrel, Ticagrelor and Ticclopidine, and (iii) GIIb/IIIa inhibitors, such as Abciximab, Tirofiban and Eptifibatide.

The dosage or concentration of the drug required to produce a favorable therapeutic effect should be less than the level at which the drug produces toxic effects and greater than the level at which non-therapeutic results are obtained. This applies to an antiproliferative agent, a prohealing agent, or any other active agent included in any of the various embodiments of the invention. Therapeutically effective dosages can also be determined from an appropriate clinical study, such as but not limited to, a Phase II or Phase III study. Effective dosages can also be determined by the application of an appropriate pharmacokinetic-pharmacodynamic model in human, or other animals. Standard pharmacological test procedures to determine dosages are understood by one of ordinary skill in the art. In some embodiments, the stent has a drug content of from about 5 μg to about 500 μg. In some embodiments, the stent has a drug content of from about 100 μg to about 160 μg. In one embodiment, the content of the drug in the drug-containing layer is from 0.5-50% by weight. In other embodiments, the drug-containing layer comprises from 0.5-10 ug/mm2 of drug (e.g., 1.4 ug/mm2).

When the drug eluting stent (1) is implanted into the human body vessel, the drug (4) may be completely released from drug-containing coating (3) within 30 days. Alternatively, for example, the drug may be completely released within 45 days, 60 days, or 120 days. In another embodiment, the drug may be completely released from the stent between 10 to 20 days, 30 days (or more than 30 days), 40 days, 45 days, 60 days, etc or more, and less than 100 days. The rate of drug release may be measured through a standard PK animal study, in which the fluid samples and tissues and the stents are extracted from animals at selected time points, and the concentration of drugs measured to best design the properties of the stent. See EXAMPLES. These animal studies are reasonably predictive of what happens in humans, as well understood by one of ordinary skill in the art. Moreover, in embodiments where the drug-containing coating (3) is made from a bio-degradable or bio-absorbable polymer, the polymer may be bio-degraded or bio-absorbed between 45 days and 90 days. For example, 50:50 PLGA (as described in Example 1 below) may exhibit in vivo degradation time of about 60 days.

### (5) the Biocompatible Base Layer

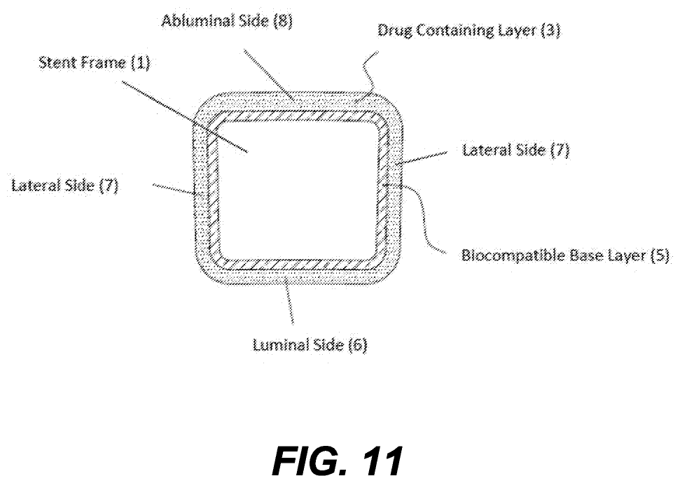

Over the stent framework (1/2), and underneath the drug-containing layer (3), a biocompatible base layer (5) may be formed, which may have a better biocompatible surface than the stent framework (2). For example, compared with a bare metal surface of the framework, the biocompatible surface of biocompatible base layer (5) may enable the early functional restoration of endothelial layers on a luminal side (6) and a lateral side (7) of the stent, which may result in a faster rate of migration and replication of the EC compared with a bare metal surface. See FIG. 11.

The biocompatible base layer (5) may be made from poly n-butyl methacrylate (PBMA), poly-methyl methacrylate, poly-acrylic acid, poly-N-[Tris(hydroxymethyl)-methyl]-acrylamide (poly-NTMA), PEDOT (poly(3,4-ethylenedioxythiophene)) PTFE, PVDF-HFP, poly(styrene-b-isobutylene-b-styrene), Parylene C, Poly-DopAmine (PDA), PVP, PEVA, SBS, PC, TiO2 or any material has good biocompatibility (or combinations thereof). In one embodiment, the base layer comprises or consists essentially of Poly Butyl MethAcrylate (PBMA).

In some embodiments, the biocompatible base layer is applied to the stent framework through a process of electrografting. The electro-grafted layer may function as an adhesion primer for the drug-containing layer (3) (e.g., during manufacturing, crimping and/or stenting). The electro-grafted primer coating may be uniform. This layer may have a thickness between 10 nm and 1.0 micron, e.g., between 10 nm and 0.5 micron or between 100 nm and 300 nm. Such a thickness may ensure that the coating does not crack. Electro-grafted layers are often capable of preventing the cracking and delamination of biodegradable polymer layers, and often exhibit equal, if not faster functional re-endothelialization (or functional restoration of the endothelium over the tent strut), than stainless steel BMS (see ref: link.springer.com/article/10.1007/s13239-021-00542-x). Furthermore, the use of an electro-grafted layer having a thickness of at least about a few tens or of a hundred nanometers may secure a good reinforcement of adhesion of the drug-containing layer (3) on account of interdigitation between the two polymeric layers. Accordingly, the choice of the nature of the electro-grafted polymer may be based upon the nature of the release matrix polymer, which itself may be chosen on the basis of the loading and kinetics of the desired drug release. In some embodiments, the electro-grafted polymer and the release matrix polymers may be at least partially miscible in order to constitute a good interface. This is the case when, for example, the two polymers have close solubility or Hildebrand parameters, or when a solvent of one of the polymers is at least a good swellant to the other.

In general, the electro-grafted polymer may be chosen from polymers known to be biocompatible. For example, the polymers may be chosen from those obtained via propagation chain reaction, such as vinylics, epoxides, cyclic monomers undergoing ring opening polymerization, or the like. Accordingly, poly-Butyl MethAcrylate (PBMA), poly-Methyl MethAcrylate (PMMA), or poly-EpsilonCaproLactone (PCL) may be used. Alternatively or concurrently, Poly-HydroxyEthyl MethAcrylate (PHEMA) may also be used.

The electro-grafted layer, (e.g., a eG™ PBMA layer) may further have a passivating behaviour and may block the release of heavy metal ions (e.g., in the blood flow or in the artery walls) from the stent framework. Said heavy metal ions may contribute to the initial inflammation caused by the introduction of the metal stent in the blood, which may provoke the partial oxidization of any metal until Nernst equilibrium is reached. In particular, the thickness of the artery walls of the electro-grafted layer and biodegradable (with no drug) branch are usually smaller than those of the bare metal stent branch, evidencing less granuloma, i.e., less inflammation.

In one embodiment, the electro-grafted layer may be biodegradable, and thus may disappear from the surface of the stent after the drug-containing layer has also disappeared.

The electro-grafted layer may have a non-thrombotic (or thromboresistant) effect and a pro-healing effect (e.g., promoting the proliferation and migration of cells, such as SMC and EC which are essential for functional restoration of the neointima or endothelium). If the cells start proliferating on the top of the drug-containing layer (e.g., before it has fully disappeared), hydrolysis of the biodegradable polymers may nevertheless continue underneath, and the cells may eventually contact the electro-grafted layer. Such a pro-healing effect may be similar to that of the stent framework if the electro-grafted layer is biodegradable itself. The pro-healing effect may be greater with a biostable electro-grafted layer that secures proper recolonization by SMC and ECs in the longer term (see ref: link.springer.com/article/10.1007/s13239-021-00542-x). Neointima or endothelium includes three parts: SMC as the bottom layer, extracellular matrix as the middle layer, and single layer of EC cells on the top of the matrix. The proper growth of all three parts are essential for the ECs layer to become fully functional, for example acting as a barrier and regulator for cellular functions of the endothelium. SMC over proliferation, on the other hand will interfere with the functional restoration of the endothelium.

In some embodiments, the electro-grafted layer may additionally be made of anti-fouling materials, and in particular of hydrophilic polymers.

The polymers which may be used as electro-grafted coating mention including, but are not limited to, vinyl polymers, such as polymers of acrylonitrile, of methacrylonitrile, of methyl methacrylate, of ethyl methacrylate, of propyl methacrylate, of butyl methacrylate, of dodecyl methacrylate, of hydroxyethylmethacrylate, of hydroxylpropylmethacrylate, of cyanoacrylates, of acrylic acid, of methacrylic acid, of 2-methacryloyloxyethyl phosphorylcholine (MPC), of trimethylsilyl-propyl-methacrylate, of styrene and of its derivatives, of N-vinylpyrrolidone, of vinyl halides, of N-[Tris(hydroxymethyl)-methyl]-acrylamide, of ethylene oxide, of molecules containing a cleavable ring such as lactones and, in particular, F-caprolactone, of lactides, of glycolic acid, of ethylene glycol, as well as polyamides, polyurethanes, poly(orthoesters), polyaspartates, or the like.

In some embodiments, the electro-grafted coating may be a vinylic polymer or copolymer, such as poly butyl methacrylate (poly-BUMA), poly hydroxyethylmethacrylate (poly-HEMA), poly 2-methacryloyloxyethyl phosphorylcholine/butyl methacrylate (poly-MPC/BUMA), poly-methacryloyloxyethyl phosphorylcholine/dodecyl methacrylate/trimethylsilylpropylmethacrylate (poly-MPC/DMA/TMSPMA), or the like. In certain aspects, the electro-grafted coating may be a biodegradable polymer, such as a polycaprolactone, a polylactide (PLA) or a polyglycolactide (PLGA).

### Adhesion Between the Electro-Grafted Coating and the Biodegradable Layer (Drug-Containing Layer or Topcoat Layer)

The drug-containing layer may adhere onto the electro-grafted layer by interpenetration of pre-formed biodegradable polymer inside the pre-made electro-grafted “brush-like” layer. This mechanism leads to the formation of an inter-phase containing both the chains of the electro-grafted layer and the chains of the drug-containing layer. The mechanism of formation of this interphase is termed “interdigitation” (see ref: iopscience.iop.org/article/10.1209/epl/i2004-10239-9). Interdigitation generally relates to the fact that the polymeric chains of the biodegradable polymer may “creep” or “reptate” inside the electro-grafted layer and may form at least one “loop” inside the electro-grafted layer. For a polymer, one “loop” may refer to the typical size of a chain when in a random configuration and may be evaluated using the radius of gyration of the polymer. Generally, the radius of gyration of a polymer is smaller than 100 nm, suggesting that, to enable improved adhesion, electro-grafted layers may be thicker than this threshold value to be capable of hosting at least one loop of the polymer(s) of the drug-containing layer.

In embodiments using interdigitation, the electro-grafted layer may be thicker than about 100 nm, may have a wettability (e.g., hydrophobic/hydrophilic) identical to that of the polymer(s) of the drug-containing layer, may have a glass transition temperature smaller than that of the polymer(s) of the drug-containing layer, and/or may be at least partially swollen by a solvent of the polymer(s) of the drug-containing layer or by a solvent containing a dispersion of the polymer(s) of the drug-containing layer.

In some embodiments, interdigitation may be caused by spreading a solution containing the drug-containing layer (and optionally the drug) over a stent framework coated with an electro-grafted layer. For example, the drug-containing layer may comprise PLGA may be dissolved in dichloroethane, dichloromethane, chloroform, or the like, optionally with a hydrophobic drugs such as Sirolimus, Paclitaxel, ABT-578, or the like. In such an example, the electro-grafted layer may comprise p-BuMA.

In some embodiments, this spreading may be performed by dipping or by spraying. In embodiments where spraying is used, a nozzle spraying the above solution may face the stent framework, which may rotate in order to present all outside surfaces to the spray. In certain aspects, the solution to be sprayed may have a low viscosity (e.g., <1 cP, the viscosity of pure chloroform being about 0.58 cP), the nozzle may be at short distance from the rotating stent, and the pressure of the inert vector gas (e.g., nitrogen, argon, compressed air, or the like) in the nozzle may be less than 1 bar. These conditions may lead to the nebulization of the liquid into small droplets of liquid, which may travel in the spraying chamber atmosphere to hit the surface of the electro-grafted layer of the stent. In embodiments where the electro-grafted polymer layer and the spray solution have the same wettability, the droplet may exhibit a very low contact angle, and the collection of droplets on the surface may therefore be filmogenic. Such a spray system may enable the manufacturing of coated stents with very little webbing in between the struts.

The relative movement of the nozzle with respect to the stent may enable the deposition of a uniform and/or relatively thin (e.g., <1 μm) layer in a single shot. The rotation and/or air renewal may enable the evaporation of the solvent, leaving the polymer layer (optionally including the drug) on the surface. A second layer may then be sprayed on the first one and so on, in order to reach a desired thickness. In embodiments where several sprays are used to reach the desired thickness, the “low pressure” spray system may be implemented in batches, in which several stents rotate in parallel with one nozzle spraying over each and every stent sequentially, therefore enabling the other stents to evaporate while another one is being sprayed.

In addition to these embodiments, the manufacturing process can comprise any of the methods of manufacturing disclosed in US20070288088 A1, which is incorporated herein by reference.

### Other Materials

All embodiments may also include additional components such as, but not limited to, lubricating agents, fillers, plasticizing agents, surfactants, diluents, mold release agents, agents which act as active agent carriers or binders, anti-tack agents, anti-foaming agents, viscosity modifiers, potentially residual levels of solvents, and potentially any other agent which aids in, or is desirable in, the processing of the material, and/or is useful, or desirable, as a component of the final product, or if included in the final product.

### Drug Release Pharmacokinetics

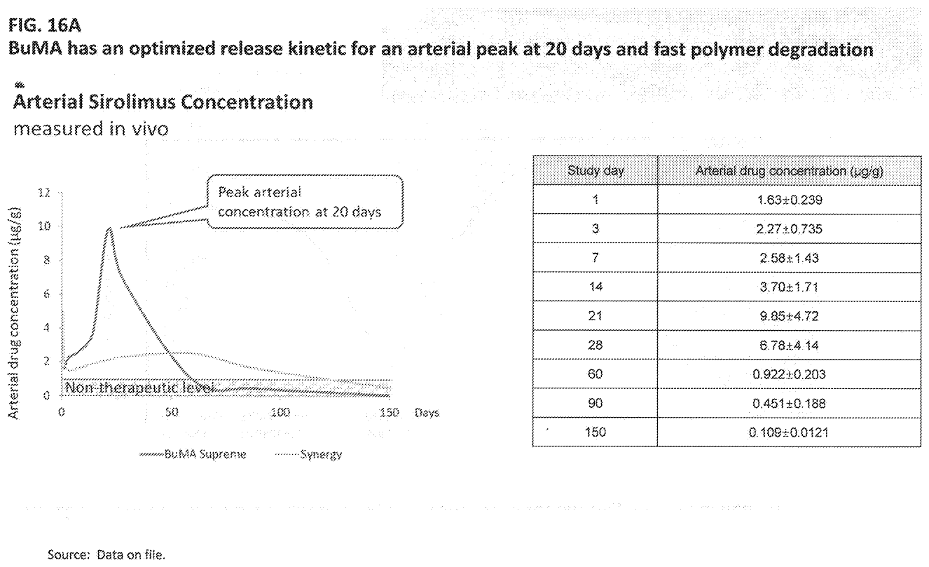

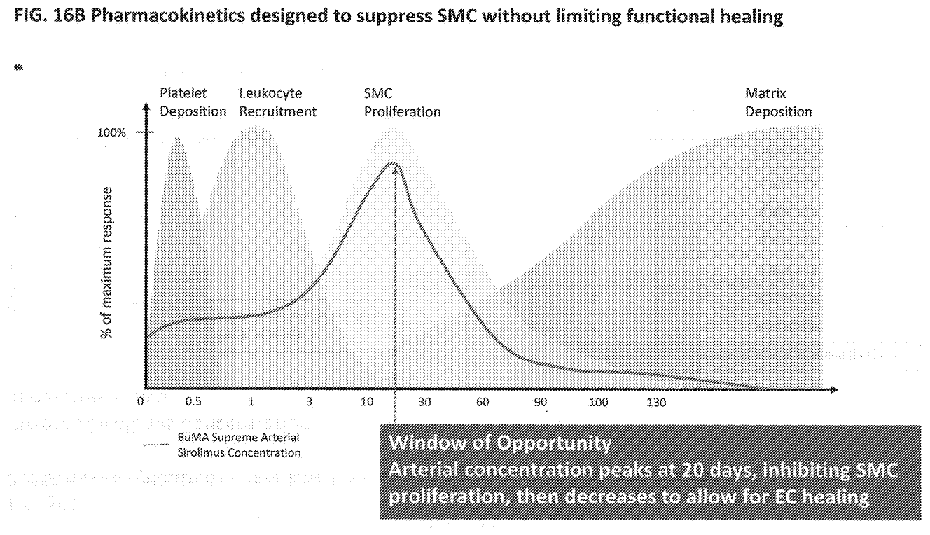

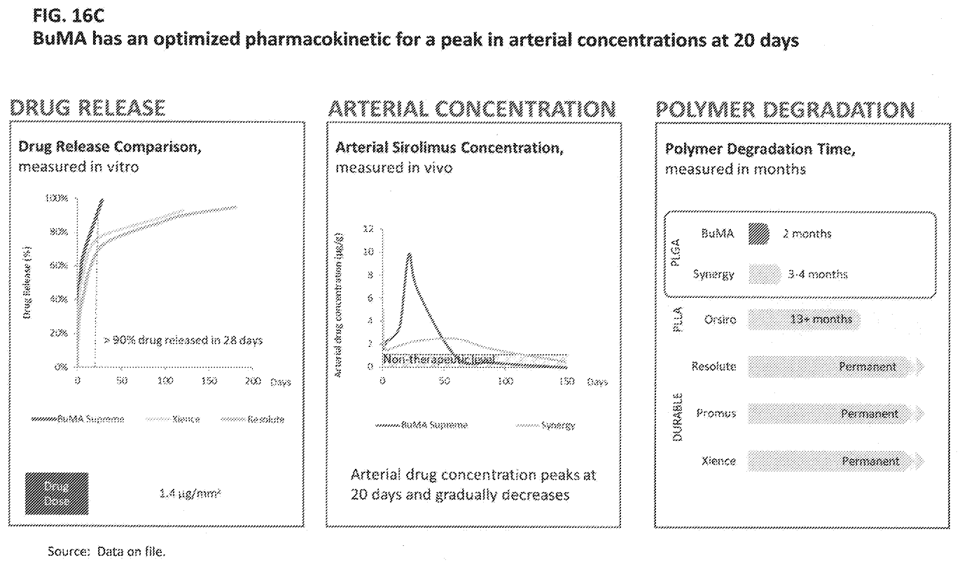

The drug release profile of the stents of the disclosure may be described in terms of release to the blood and/or release into the arterial tissue adjacent to the stent. Example 3 provides a pharmacokinetic study of a stent according to the disclosure. FIG. 16 and Table 3 provide preferred embodiments for the pharmacokinetic values associated with a stent with a desirable performance according to the preferred embodiments of the disclosure. In one embodiment, the arterial concentration of the drug follows the release profile in FIG. 16B, peaking around day 20 post stent implantation. This release profile is an ideal profile if for a drug that reduces SMC proliferation because this is the same period of time at which the proliferation of SMC would reach a maximum without a drug in the stent. This release profile for a SMC proliferation inhibitor does not reduce endothelial vascular restoration.

In one embodiment, more than approximately 85-95% of the drug is released from the stent in vitro in approximately 28 days. In one embodiment, this pharmacokinetics is achieved with approximately 1-2 ug/mm² of drug in the stent. In one embodiment, the same stent has a drug-containing layer comprising PLGA and degrades in approximately 2 months. In one embodiment, the same stent has the drug release profile of FIG. 16C, and the arterial drug concentration peaks at about 20 days and gradually decreases. In one embodiment, the drug is sirolimus.

Table 4 shows some preferred pharmacokinetics exhibited by the stents of the disclosure. In one preferred embodiment, the stent (for example, the drug-containing layer) is designed to provide the following drug release profile in the treated left anterior descending artery (LAD) tissue: time of maximum observed concentration (Tmax)=500 hours; maximum concentration achieved (Cmax) is 11.0 ng/g, and the AUClast 6580 hr*microgram/gram. In one embodiment, the stent (primarily the drug-containing layer) is designed to provide the following drug release profile in the right coronary artery (RCA) tissue: Tmax=670 hours; Cmax is 9.23 ng/g, and the AUClast is 8240 hr*microgram/gram. See examples. In some embodiments, these pharmacokinetic profiles are achieved by manipulating the amount/thickness and/or composition of the drug-containing layer. In some embodiments, these pharmacokinetic profiles are achieved by manipulating the amount and/or identity of the drug.

In one embodiment, the Tmax in the LAD and/or RCA tissue is between 200-300 hours, 300-400 hours, 400-500 hours, 500-600 hours, 600-700 hours, 700-800 hours, 800-900 hours, 900-1000 hours. In one embodiment, the Tmax in the LAD and/or RCA tissue is 100 hours or less, 200 hours or less, 300 hours or less, 400 hour or less, 500 hours or less, 600 hours or less, 700 hours or less, 800 hours or less, 900 hours or less, or 1000 hours or less. In one embodiment, Cmax in the LAD and/or RCA tissue is between 1-5, 6-10, 9-15, 11-15, 16-20, 21-25, 26-30, 31-40, 41-50, 51-55, 55-60, so one and so forth until 95-100 ng/g. In one embodiment, the Cmax in the LAD and/or RCA tissue is 5 ng/g or less, 10 ng/g or less, 15 ng/g or less, 20 ng/g or less, 25 ng/g or less, 30 ng/g or less, 35 ng/g or less, 40 ng/g or less, 45 ng/g or less, 50 ng/g or less, 55 ng/g or less, 60 ng/g or less, so one and so forth until 100 ng/g or less. In one embodiment, the AUClast in the LAD tissue is between 100-1000, 1000-2000, 2000-3000, 3000-4000, 4000-5000, 5000-6000, 6000-7000, 7000-8000, 8000-9000 hr*microgram/gram. In one embodiment, the AUClast in the LAD tissue is 1000 or less, 2000 or less, 3000 or less, 4000 or less, 5000 or less, 6000 or less, 7000 or less, 8000 or less, 9000 or less, 1000 or less, 1000 or less hr*microgram/gram. In some embodiments, these pharmacokinetic profiles are achieved by manipulating the amount/thickness and/or composition of the drug-containing layer. In some embodiments, these pharmacokinetic profiles are achieved by manipulating the amount and/or identity of the drug. In some embodiments, both of these aspects of the stent are manipulated in a coordinated fashion.

Exemplary Stent Characteristics after Implantation in the Rabbit Iliac Artery

In one embodiment, it was also discovered that a stent with the following characteristics is expected to have superior efficacy and/or safety performance relative to stents with different values for the following parameters as evaluated in the rabbit iliac stent model:
the uptake of Evans' Blue dye by the artery in the stented zone is <40% at 45 days and <25% at 90 days, post-stent implantation;

The ratio R, measured by confocal microscopy in a longitudinal cross-section of the stented zone of the stented artery, of the quantity of P120 protein to that of VE-Cadherin (R=[P120]/[VE-cad]), which characterizes the degree of co-localization of the said proteins in the scaffolded region, is higher than 70% at 45 days, and higher than 80% at 90 days after implantation; and

The cell shape index I, defined as the ratio between the largest length [a] of endothelial cells observed by confocal microscopy divided by the size [b] in the direction perpendicular to said longest length (I=[a]/[b]), is larger than 2 at 45 days after implantation, and larger than 3.5 at 90 days after implantation. All values may be modified by the term about.

See Examples. Accordingly, the disclosure provides a stent with a structural design in terms of, for example, stent framework, drug-containing layer, drug, and/or biocompatible base layer, such that the stent presents one or more of those parameters. Again, these newly discovered parameters serve as new guides for stent design and improvement.

### Methods of Using the Stents:

In one embodiment, a stent is a medical device used for improving a stenosed region or an occluded region in a lumen in an organism such as a blood vessel, a bile duct (often, plastic stents) a trachea, an esophagus, an airway, an urethra or the like. Stents are inserted into these and other hollow organs to ensure that these hollow organs maintain sufficient clearance.

One use for medical stents is to expand a body lumen, such as a blood vessel, which has contracted in diameter through, for example, the effects of lesions called atheroma or the occurrence of cancerous tumors. Atheroma refers to lesions within arteries that include plaque accumulations that can obstruct blood flow through the vessel. Over time, the plaque can increase in size and thickness and can eventually lead to clinically significant narrowing of the artery, or even complete occlusion. When expanded against the body lumen, which has contracted in diameter, the medical stents provide a tube-like support structure inside the body lumen. Stents also can be used for the endovascular repair of aneurysms, an abnormal widening or ballooning of a portion of a body lumen which can be related to weakness in the wall of the body lumen. Stents may also be used to treat neurological diseases involving the neurological vasculature.

Stents are used not only for mechanical intervention but also as vehicles for providing biological therapy. Biological therapy uses medicated stents to locally administer a therapeutic substance. The therapeutic substance can also mitigate an adverse biological response to the presence of the stent. A medicated stent (i.e., a stent comprising a drug) may be fabricated by the methods disclosed herein to include a polymeric carrier that includes an active or bioactive agent or drug.

In one embodiment, the stent is used in methods of treating a disease or disorder in a subject. Examples of disease or disorders where the stent can be used include diseases of the vasculature (heart disease, thrombosis), tumors, angioma, obstruction of lacrimal gland and other diseases of a lumen. The stent can be used for percutaneous coronary intervention (PCI) as well as in peripheral applications, such as the superficial femoral artery (SFA). In some embodiments, the stent can be used for the treatment of angiostenosis or to prevent restenosis, by utilizing a cell proliferation-suppressing agent such as cytostatic (e.g., paclitaxel) or immunosuppressant as the drug. In some embodiments, a ureteral stent of the disclosure is introduced into the kidney and/or the bladder of a subject.

As used herein, the term “subject” refers to human and non-human animals, including veterinary subjects. The term “non-human animal” includes all vertebrates, e.g., mammals and non-mammals, such as non-human primates, mice, rabbits, sheep, dog, cat, horse, cow, chickens, amphibians, and reptiles. In a preferred embodiment, the subject is a human and may be referred to as a patient.

As used herein, the terms “treat,” “treating” or “treatment” refers, preferably, to an action to obtain a beneficial or desired clinical result including, but not limited to, alleviation or amelioration of one or more signs or symptoms of a disease or condition, diminishing the extent of disease, stability (i.e., not worsening) state of disease, amelioration or palliation of the disease state, diminishing rate of or time to progression, and remission (whether partial or total), whether detectable or undetectable. “Treatment” can also mean prolonging survival as compared to expected survival in the absence of treatment. Treatment does not need to be curative.

Methods of Introducing the Stent into the Subject

In one embodiment, the stent is introduced into the subject body via a catheter, or by implantation. In other embodiments, the stent is introduced by balloon catheter

The terms “inserting a stent”, “delivering a stent”, “placing a stent”, “employing a stent”, and similar expressions as described herein all mean introducing and transporting a stent through a bodily lumen into a region that requires treatment by a mechanism such as a guidewire, balloon catheter, or other delivery system for self-expanding stents. In general, it is done by positioning a stent on one end of the guidewire, inserting the end of the guidewire through the bodily lumen of a subject, advancing the guidewire in the bodily lumen to a treatment site, and removing the guidewire from the lumen. The insertion may also be facilitated by other accessories such as a delivery sheath, a push rod, a catheter, a pusher, a guide catheter, an endoscope, a cystoscope, or a fluoroscopy. Other methods of delivering a stent are well known in the art.

### The Manufacturing Process

The stents of the disclosure may be manufactured by adaptations and manipulations of manufacturing steps described in the art.

Take a metal stent frame, for example:

### 1) Stent Manufacture

The stent frame can be laser cut from a metal tubing. After the laser cutting, the stent frame will undergo an electro-polishing process to make the edge of the stent frame smooth.

### 2) Base Layer Manufacture

Place the stent frame into a reservoir full of butylmethacrylate (monomer). During the electro-grafting process, the polymerization of butylmethacrylate will be initiated by some initiators and the base layer (Poly-butylmethacrylate) will be bonded (covalent bond) on the stent frame to provide surface with a better biocompatibility.

### 3) Drug Containing Layer Manufacture

50/50 PLGA (biodegradable polymer) and Sirolimus (drug) is mixed with a certain weight ratio and dissolved in chloroform to make the spray solution. The stent frame with base layer is fixed onto a rotator and spray coated with the spray solution.

### Examples of Making the Stent Framework (2):

In some embodiments, the stent framework may comprise a pre-fabricated mesh of magnesium alloy. The alloy may be fully biodegradable between six and nine months after implantation. Additionally or alternatively, the stent framework may maintain mechanical radical strength for at least three months. Similarly, the stent framework may comprise a pre-fabricated Poly-L-lactic acid (PLLA) or other biocompatible fully biodegradable polymers. Such polymers may maintain the mechanical radical strength for at least three months.

In some embodiments, the stent framework may be cut from a metal tubing, e.g., using a laser. An electro-polishing process may smooth the stent framework after cutting.

### Examples of Making the Biocompatible Base Layer (5):

### Electrochemical Reaction

In one embodiment, n-butyl methacrylate monomer may be dissolved into N,N dimethyl formamide solvent (DMF). In certain aspects, sodium nitrate may be added as an electrolyte to increase the conductivity of the solution. The solution may be rotated and mixed for 120 minutes. In one example, the concentration of methacrylate may be 20%, the concentration of sodium nitrate may be 5.0×10⁻²M, and the concentration of DMF may be 80%.

A reactor containing the above primer layer coating solution may use an electrochemical reaction to coat the stent scaffold with the solution. For example, the stent is connected to the working electrode plug of a potentiostat, and a counter-electrode made of two foils of graphite having a surface area large (>×10) compared to the surface area of the stent, are facing each other a few centimeters apart so that the stent can be placed between the foils. A voltage comprised between 3 and 4 Volts is then applied so that the stent acts as a cathode. This voltage is usually applied in scanning voltametric mode, from 0 to 3-5 Volts and back, at a linear scanning rate of ca. 50 mV/second. An inert gas such as argon or nitrogen is bubbling in the DMF solution all along the voltametric process, which lasts approximately 120 minutes.

The biocompatible base layer may then be baked in vacuum (e.g., at 10 mbar or less). In one example, the baking may occur at approximately 40° C. for 180 minutes. A biocompatible base layer formed with this process may have a thickness of approximately 200 nm.

In other embodiments, the biocompatible base layer may be manufactured merely dipping the stent inside a bath containing some specific reactive species that can spontaneously react with the metallic alloy of the stent. This is the case for example with magnesium based alloys, which react spontaneously with 4-nitrobenzene diazonium tetrafluoroborate at 5·10⁻³ mol/l, to form a grafted layer of ca. 100-150 nanometers.

### Examples of Making the Drug-Containing Layer (3):

In one embodiment, the drug-containing layer is applied to the stent via a spray coating process. In other embodiments, the process of application of the drug-containing layer to the stent (directly or on the surface of the biocompatible base layer) comprises, for example, dipping, vapor deposition, and/or brushing.

### Spray Coating Process

### A. Process

In some embodiments, the drug-containing layer (3) may be formed using a spray coating process for disposing a polymer coating on the stent framework (or on a polymer-coated stent, e.g., a stent coated in the electro-grated coating described below). In one example, a 20 millimeter long electro-grafted stent was spray coated with biodegradable polyester (polylactide-co-glycolide 50/50, PLGA) containing Sirolimus. The copolymer (0.25% w/v) was dissolved in chloroform. Sirolimus was then dissolved in the chloroform/polymer mixture to obtain a final ratio Sirolimus/polymer of (1/5). In another example, the mixture may comprise 50/50 PLGA (e.g., 5 g) with rapamycin (e.g., 0.5 g) dissolved in chloroform (e.g., 600 mL). The mixture was then applied to the stent, mounted on rotative mandrel, by spraying with a fine nozzle with the following parameters:

Spray parameter

| | |
|---|---|
| Spraying flow (μL/s) | 24 |
| Spraying volume (μL/s) | 192 |
| Pressure (bar) | 0.3 |
| Stent rotation speed (rpm) | 2000 |
| Nozzle/stent distance (mm) | 6.5 |
| Number of spray run | 50 |

Alternatively, such parameters may be adjusted by one of ordinary skill in the art to meet the conditions of this disclosure, to produce a un-even distribution of the drug layer on the stent surface (thinner on the luminal face). In some embodiments, the parameters can be adjusted from those used in U.S. patent application Ser. No. 13/850,679 (published as 2014/0296967 A1), U.S. patent application Ser. No. 11/808,926 (published as 2007/0288088 A1), and U.S. Provisional Patent Application No. 60/812,990, all of which are incorporated herein by reference in their entireties.

The conditions of the drug spraying may be adjusted so that the drug-containing coating (3) may be applied to a luminal side (6), lateral side (7), and abluminal side (8) of the stent. See FIG. 11. Due to the high speed rotation spray and centrifugal effect, drug-containing coating (3) may have a higher (and tunable) thickness on the abluminal side (facing the vessel wall) (8) with respect to the luminal side (facing the blood flow) (6) and the lateral side (7). An embodiment of this disclosure is a stent with such an un-even coating. In one embodiment, relative high speed spinning, and low pressuring process over coating the stent with the drug-containing solution was found to produce this result. Drying at 40° C. was then performed in a vacuum oven. Using the above parameters, the coating on this example stent weighs 800+/−80 μg and has a thickness of about 5-7 μm. The drug loading in this example stent was 164+/−16 μg.

### Manufacturing of the BUMA-Supreme

BuMA Supreme was made first with a 80 μm thickness of specific designed bare metal stent, then eG coated with PBMA of about 200 nm thickness, and top coated the eG coated stent with spray coating process of drug and PLGA formulation.

### EXAMPLES

### Example 1

### In Vivo Studies in Rabbits

Stents prepared by the method just described in the immediately preceding exemplary methods were used in vivo. A first stent was prepared according to this example method with the following stent framework structure: In this example, the stent framework comprised stainless steel with a 10 crest design. This design may result in improved radial strength and greater uniformity after stent expansion as compared with designs having fewer crests. The stent (cobalt chromium) possessed the following additional characteristics: conformal coating with a drug-containing layer of biodegradable polymer (PLGA, 3.5-10 um) with 1.4 ug/mm² of Sirolimus; 80 um strut thickness; and an electrografted durable/biocompatible base layer (supporting the drug-containing layer) made of PBMA with a thickness of 100 nm-200 nm.

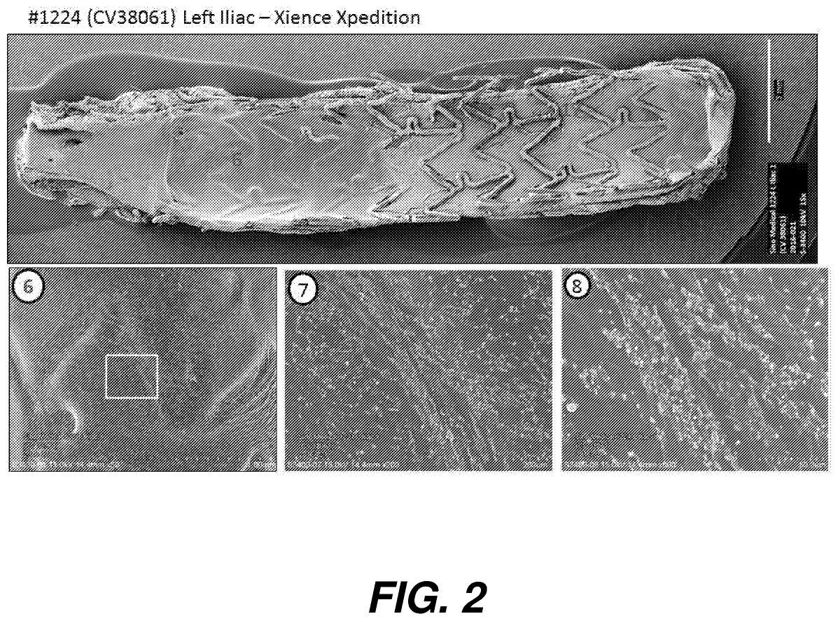

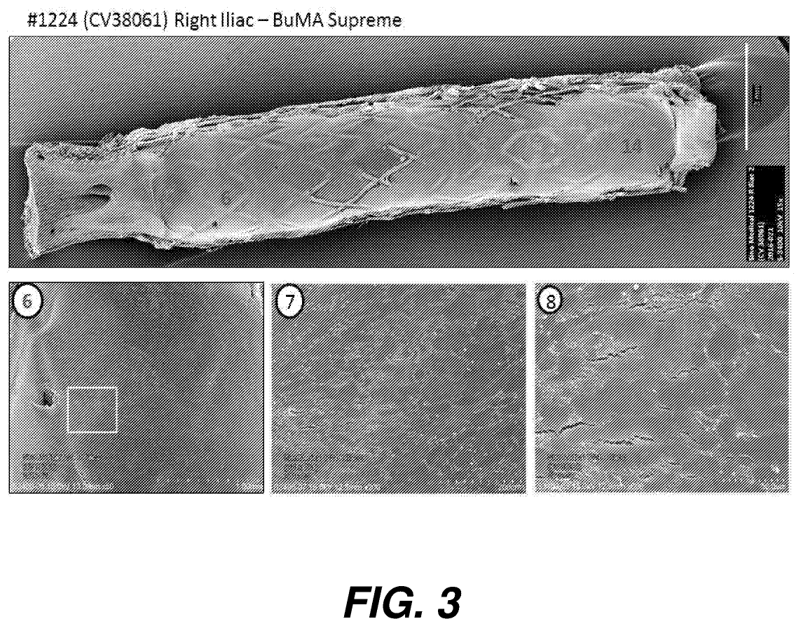

A number of stents with these properties were implanted into rabbits. All surgeries were performed using aseptic techniques. Rabbits were placed in a supine position and the hind-legs abducted and externally rotated at the hips with the knees extended. During surgery to stabilize the animal's physiologic homeostasis, animals were maintained on 0.9% Sodium Chloride, USP, intravenous drip at the rate of 10-20 ml/kg/hr and on warm water blanket. The animal's heart rate, blood pressure, body temperature, respiratory rate, O₂ saturation, CO₂ level, and the concentration Isoflurane was monitored and recorded every 15 minutes. The left and right iliac arteries were injured by balloon endothelial denudation. A 3.0 mm×8 mm standard angioplasty balloon catheter was placed in the distal iliofemoral artery over a guide wire using fluoroscopic guidance and inflated to 8 ATM with 50:50 contrast/saline. The catheter then was withdrawn proximally in its inflated state approximately to the level of the iliac bifurcation. The balloon was deflated, repositioned in the distal iliac, and vessel denudation at 10 ATM) then was repeated over the same section of vessel initially denuded. Immediately following balloon denudation, coronary stents (BuMA Supreme, Xience [Xience Xpedition], of BuMA BMS (3.0 mm×15.0 mm) were implanted in the denuded segment of the iliofemoral artery according to the scheduled allocation. The pre-mounted stent/catheter was delivered into the distal iliofemoral artery over a guide wire using fluoroscopic guidance. Stents was deployed at the suggested nominal inflation pressures (10 ATM) at a target balloon to artery ratio of ˜1.3 to 1.0 delivered over 30 seconds. Repeat angiography was performed to assess stent placement and patency. Following post-implant angiography, all catheters/sheaths were then withdrawn and surgical wound closed and the animals recovered. For example, as shown in FIG. 3, when a stent according to the present disclosure (Buma Supreme) was implanted in a rabbit for days, the stent exhibited a better endothelial coverage (80%) as compared with the Xience Xpedition depicted in FIG. 2 (50%), as assessed by scanning electron microscopy (SEM).

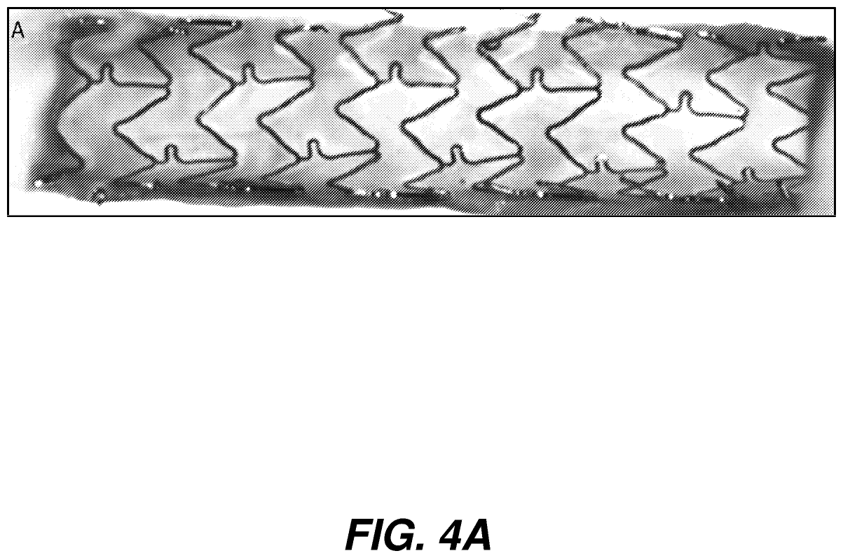

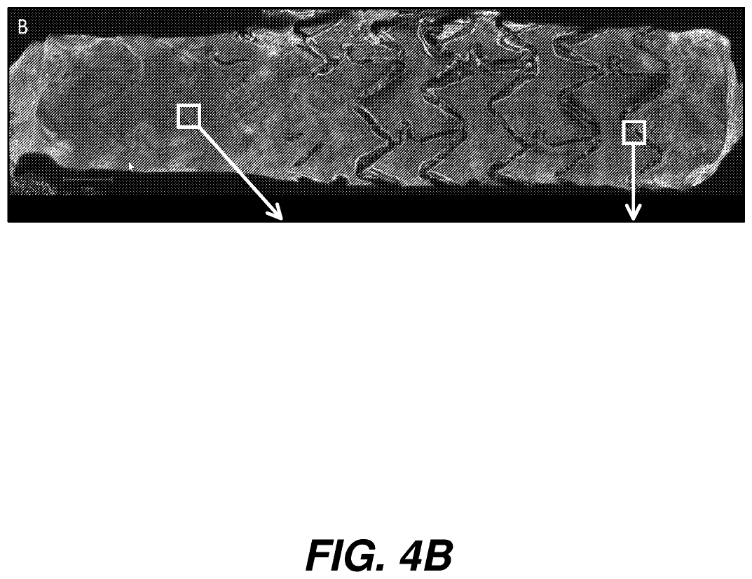

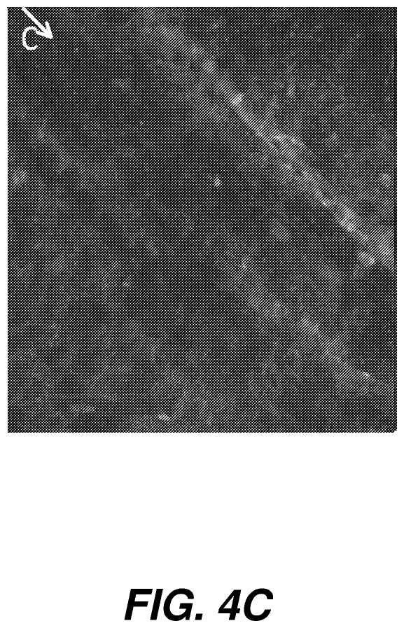

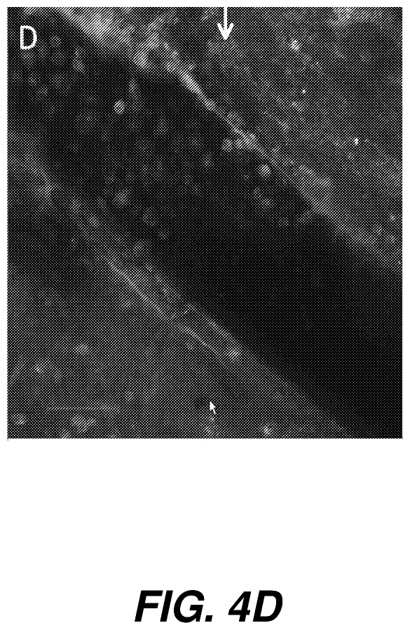

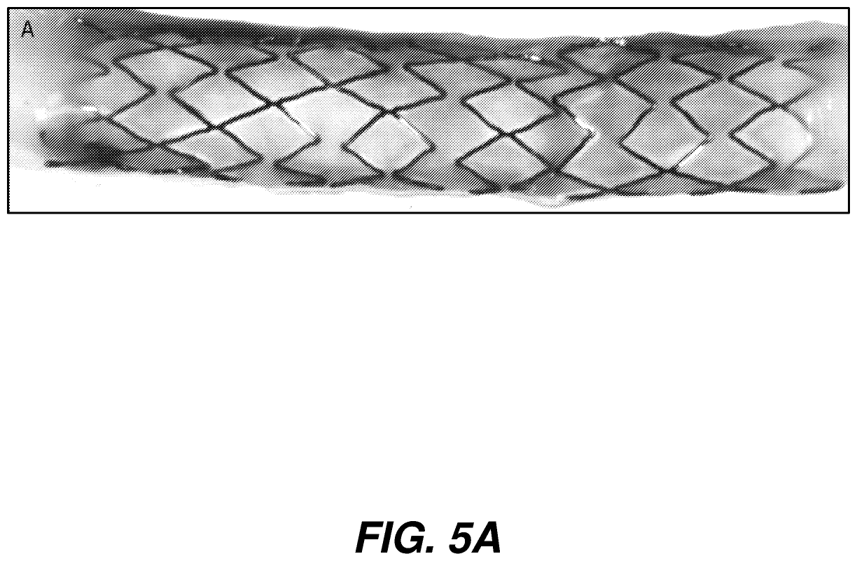

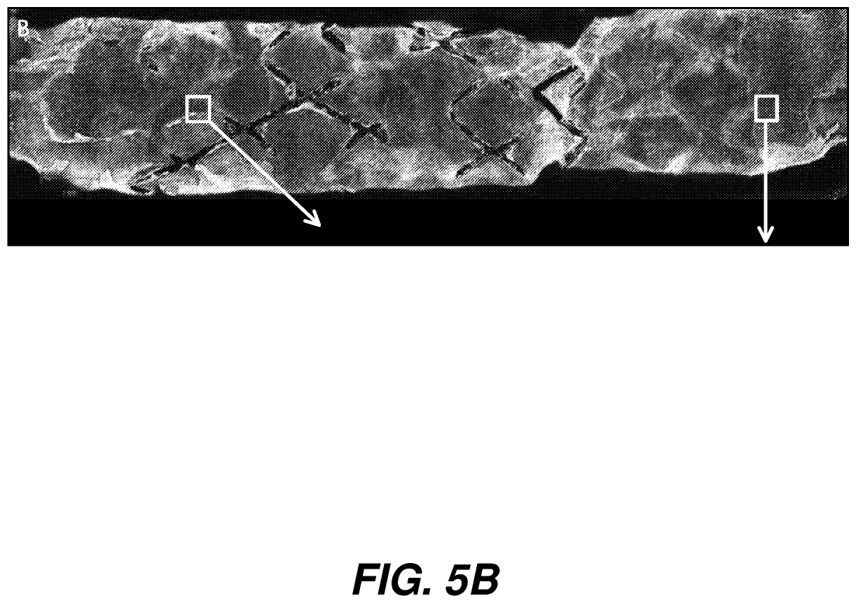

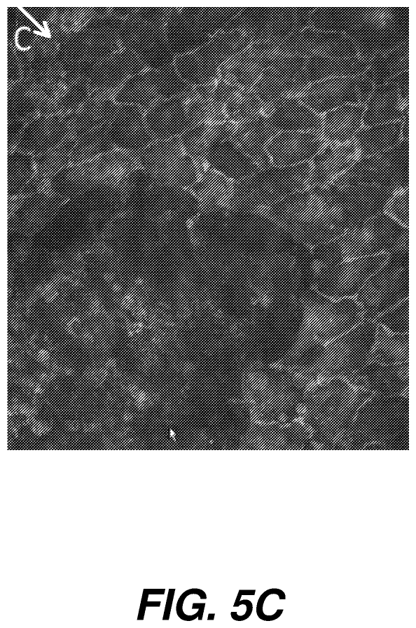

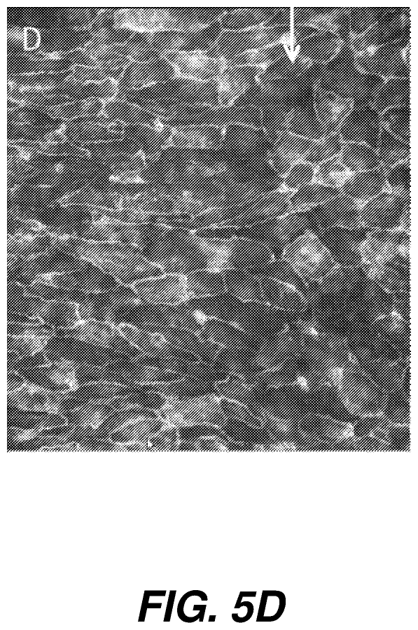

Moreover, as shown in FIGS. 5A through 5D, after 60 days of implantation in a rabbit, a stent according to the present disclosure exhibited a better functional endothelial coverage (38%) as compared with the Xience Xpedition stent depicted in FIGS. 4A through 4D (21%).

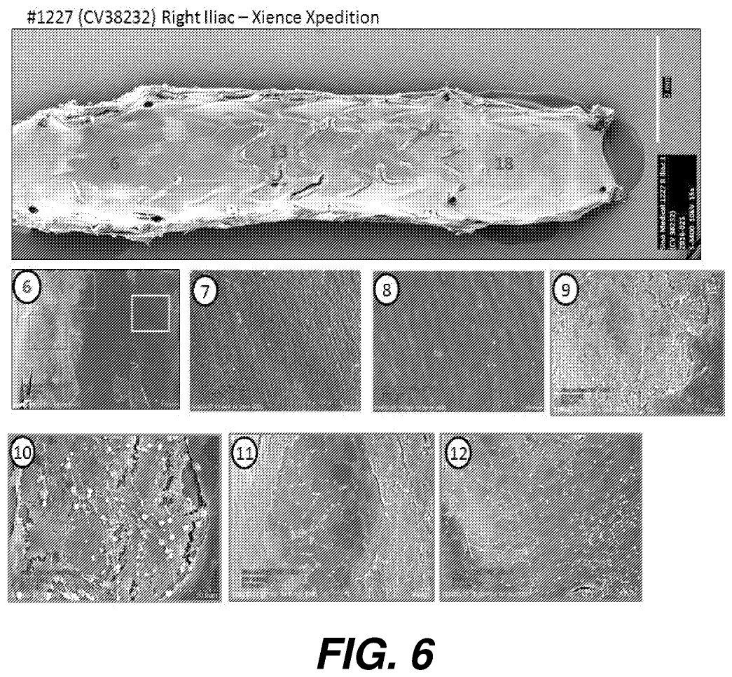

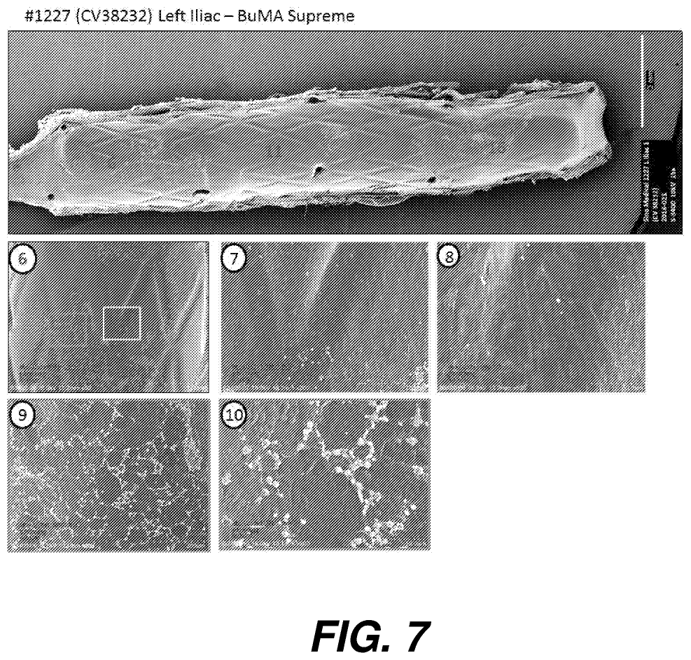

As further shown in FIG. 7, after 90 days of implantation in a rabbit, a stent according to the present disclosure exhibited a better endothelial coverage (99%) as compared with the Xience Xpedition stent depicted in FIG. 6 (70%).

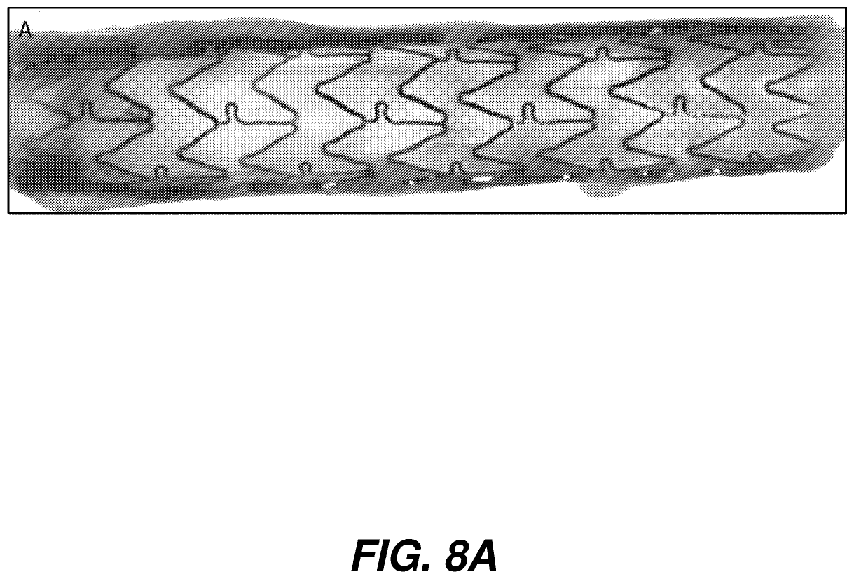

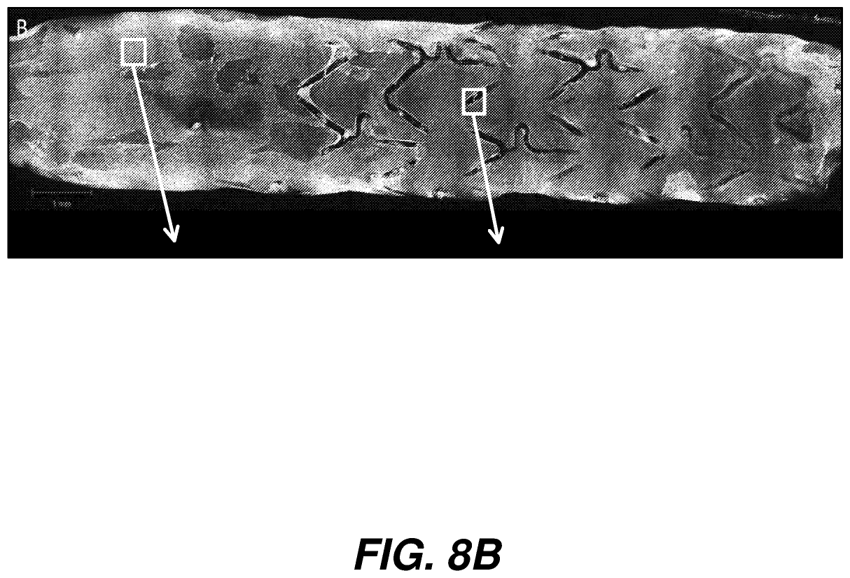

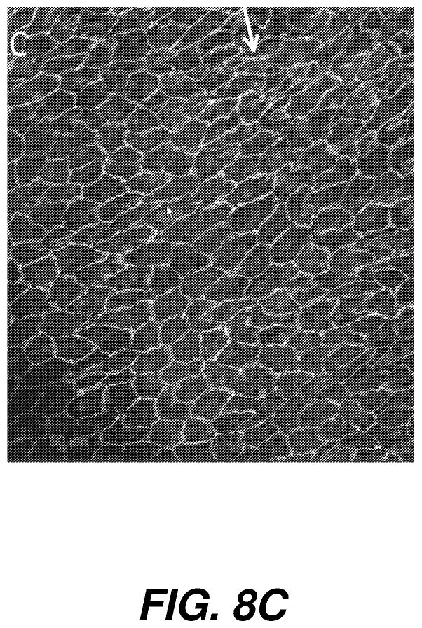

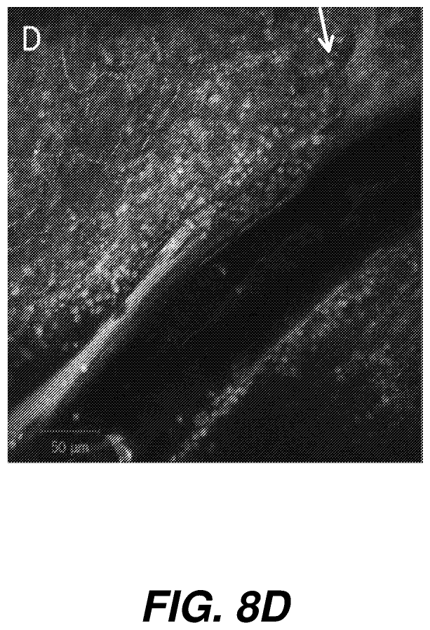

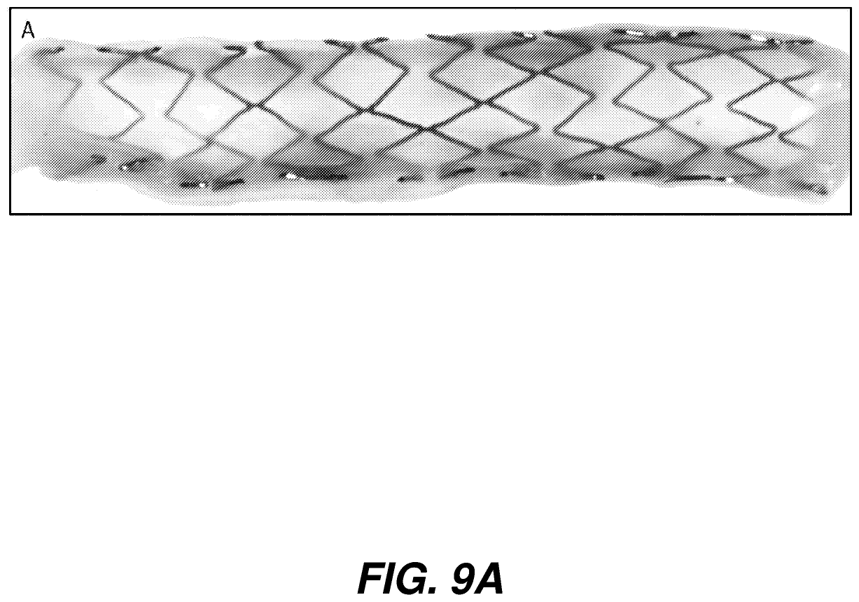

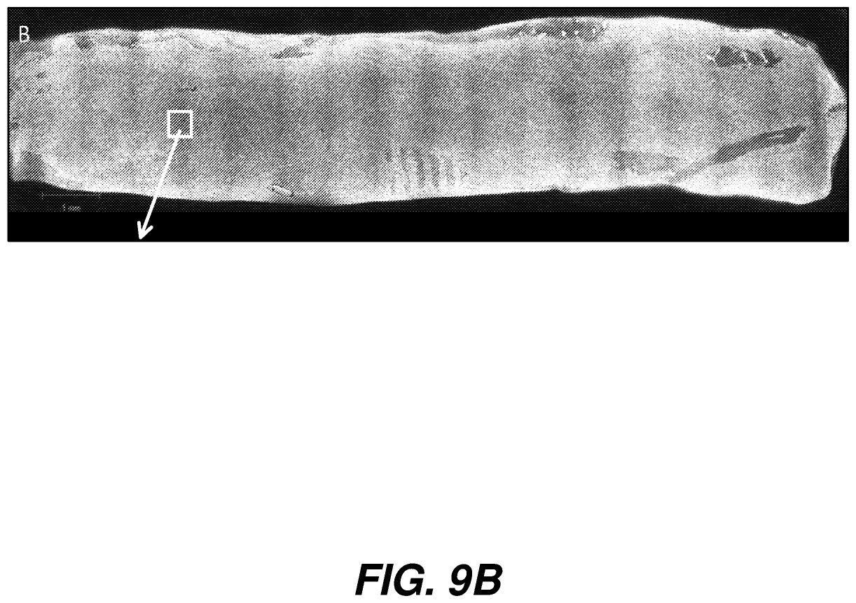

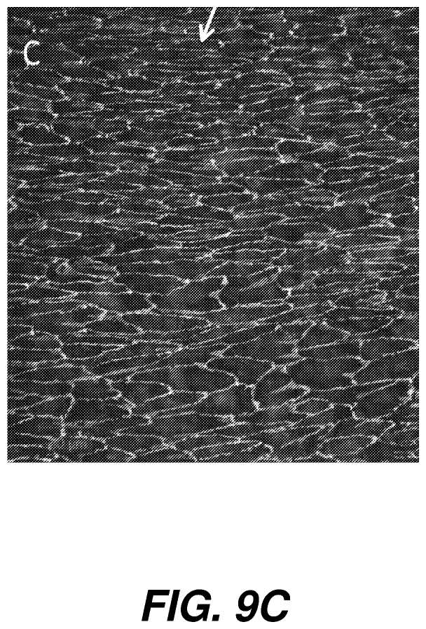

Finally, as shown in FIGS. 9A through 9C, after 90 days of implantation in a rabbit, a stent according to the present disclosure exhibited a better functional endothelial coverage (100%) as compared with the Xience Xpedition stent depicted in FIGS. 8A through 8D (46%).

A cell shape index is defined as (cell height divided by the cell width) which express the morphology of cell. At 45 days, the cell shape index of the stent according to the present disclosure was 2.69, the Xience Xpedition was 1.73; at 90 days were 3.34 and 2.20, respectively, which showed the significance difference.

A second set of experiments was prepared according to this example method with the following stent framework structure:

The stent (BuMA Supreme) was coated by the same spray coating process described above with a conformal coating of biodegradable polymer (PLGA). The strut thickness was 80 μm and the stent was made of Cobalt-chromium alloy. The eG-layer was made of PBMA (100 nm-200 nm) and the drug containing layer of PLGA (3.5 to 10 um) with 1.4 ug/mm2 of sirolimus.

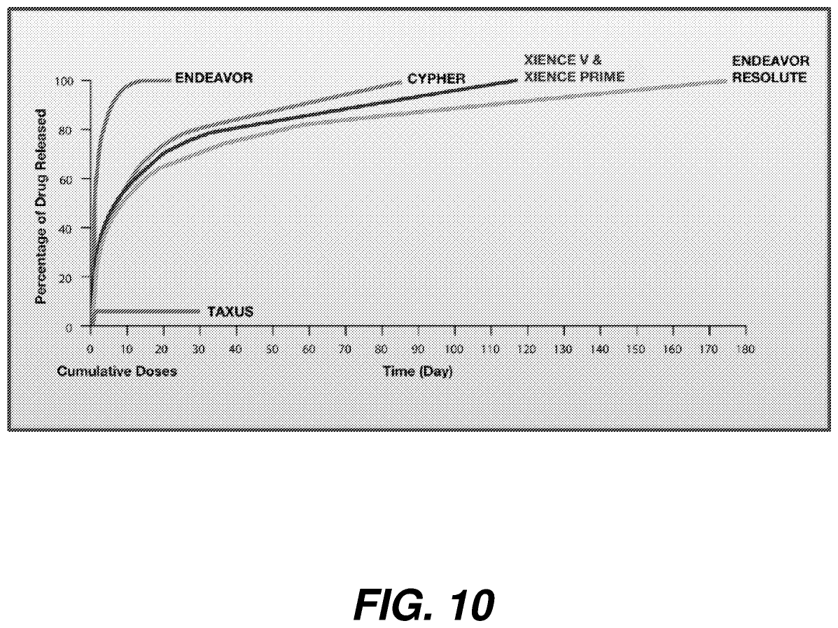

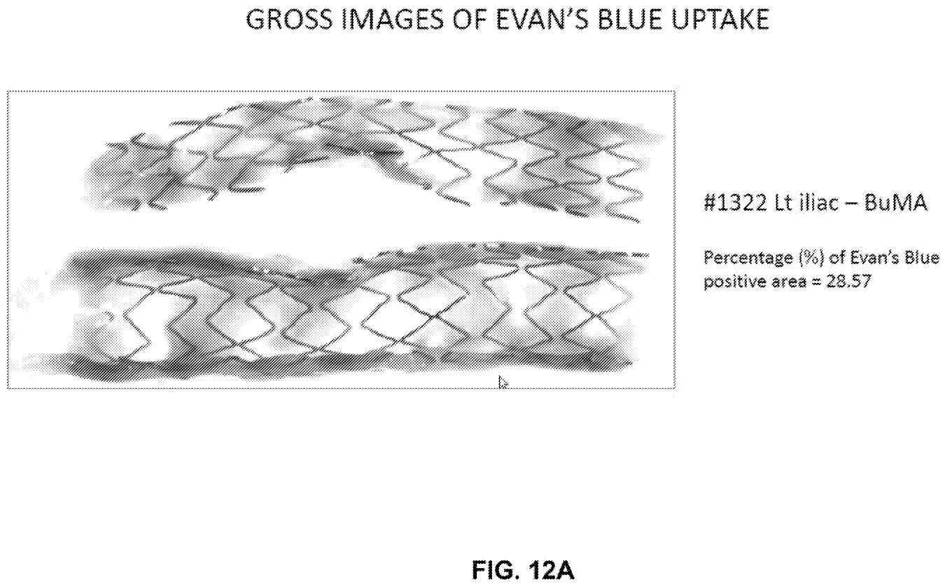

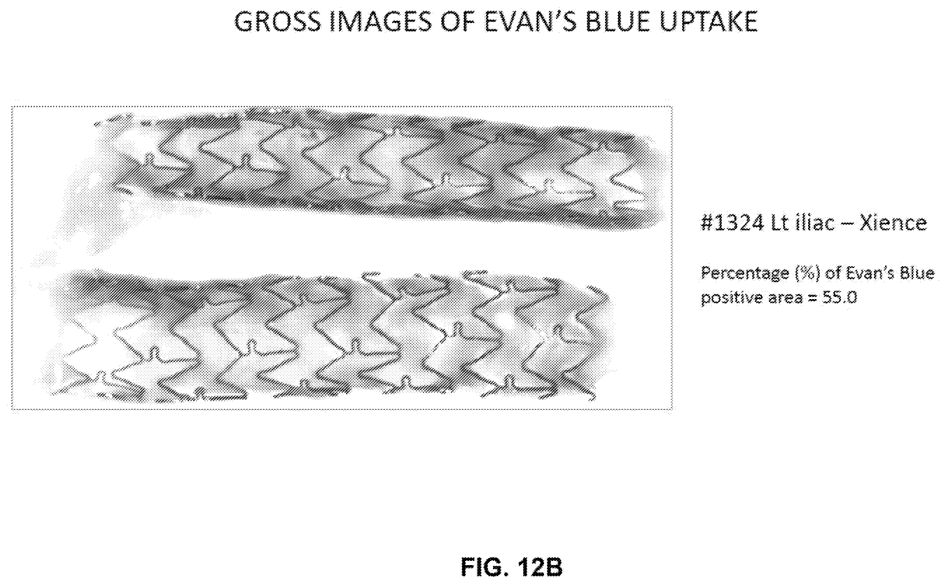

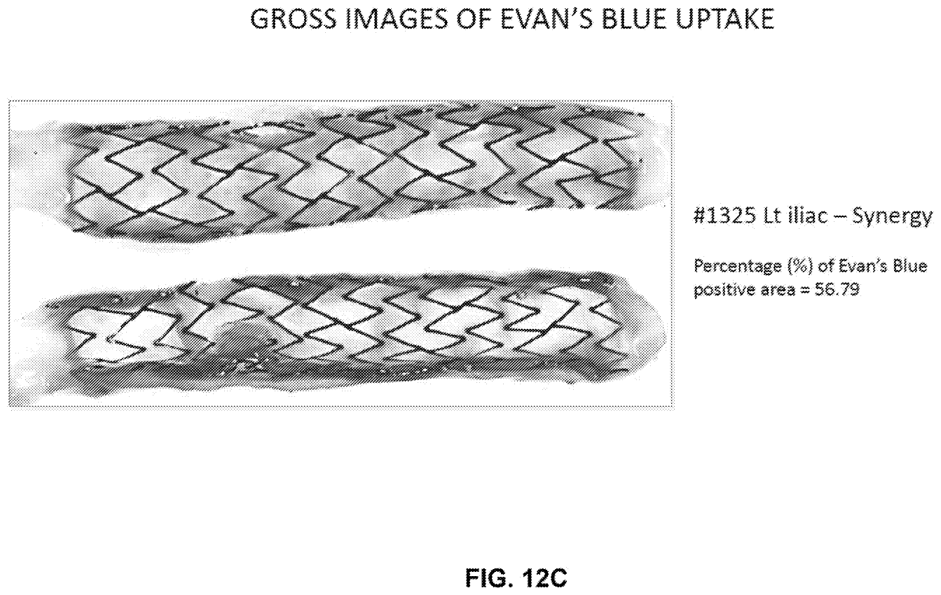

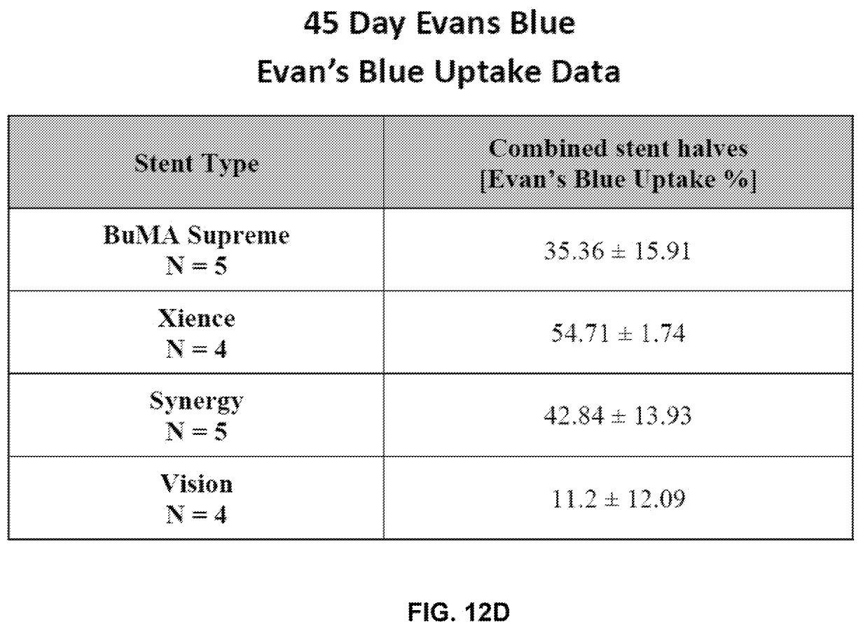

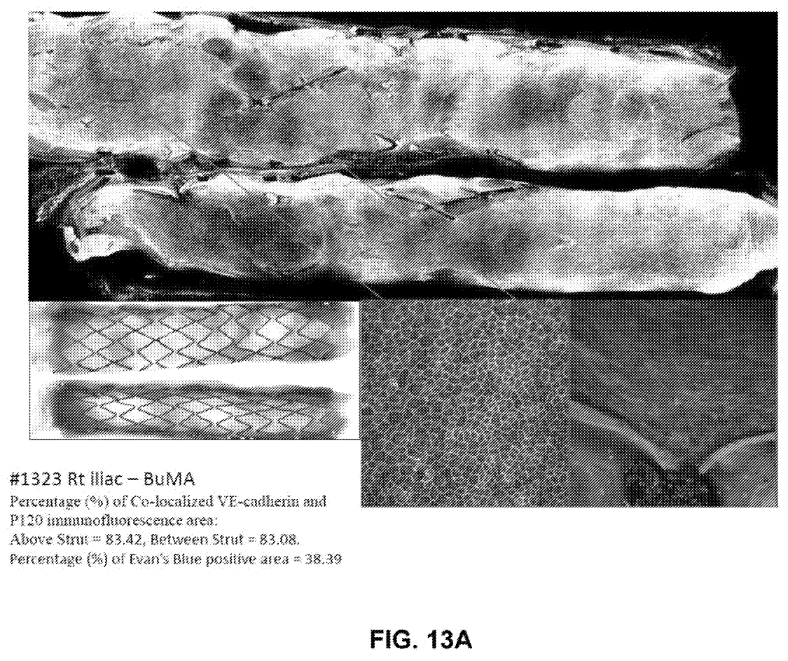

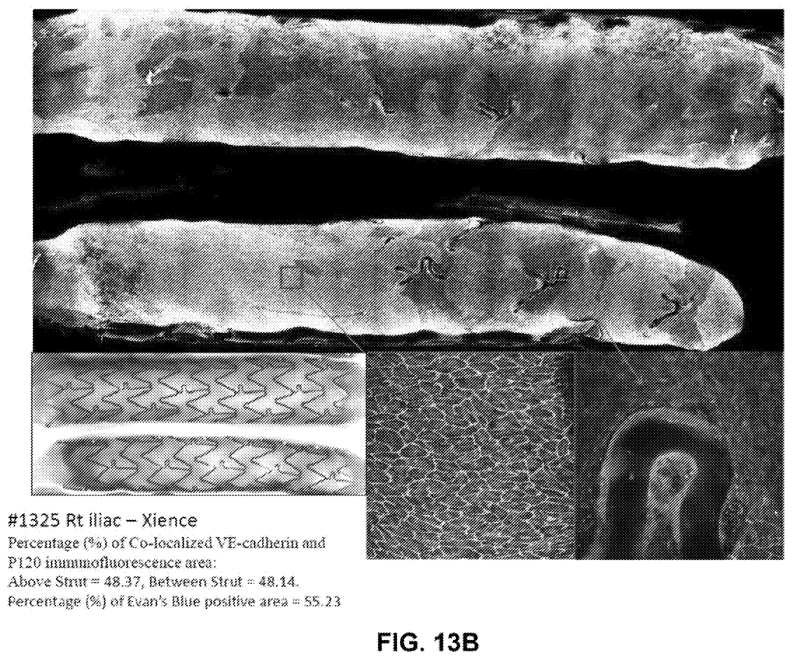

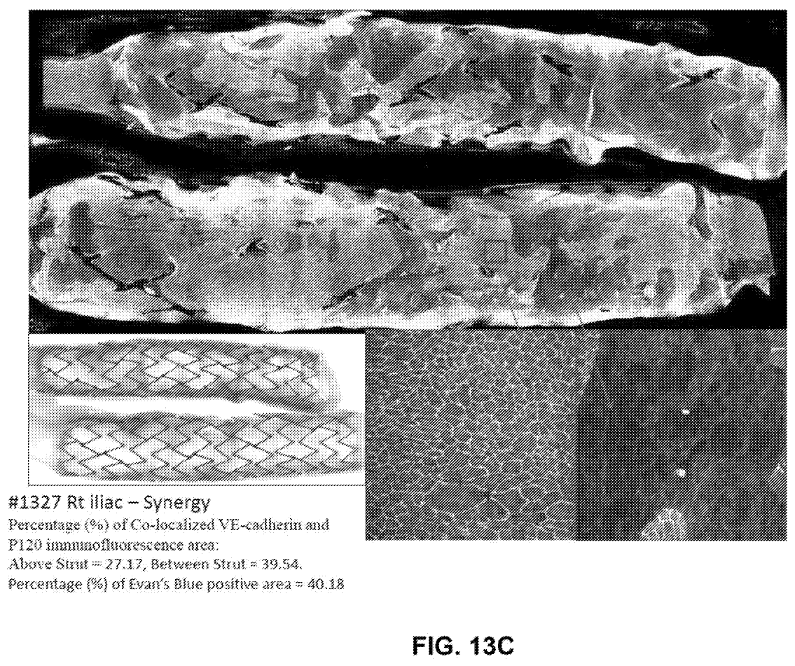

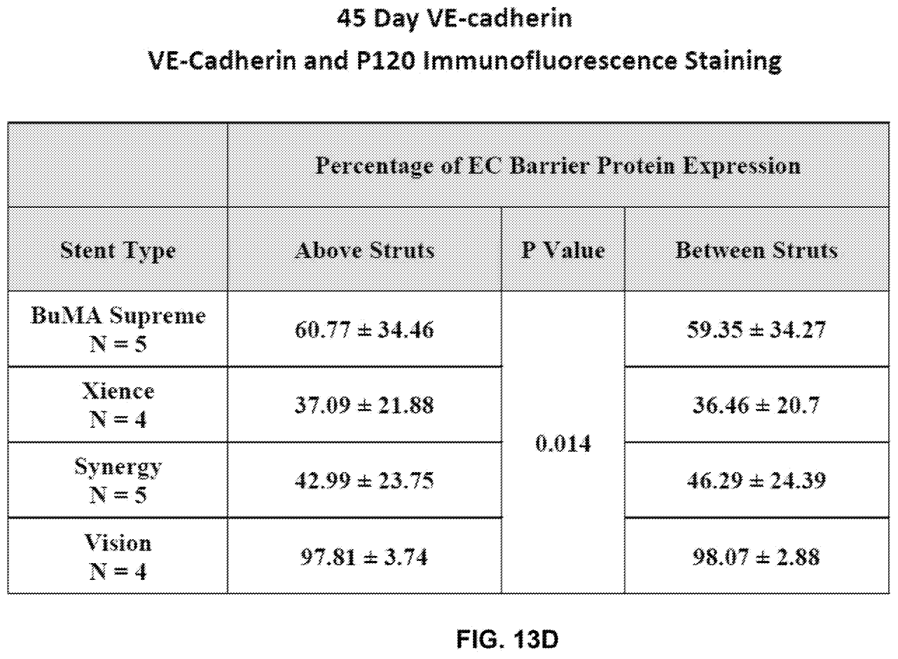

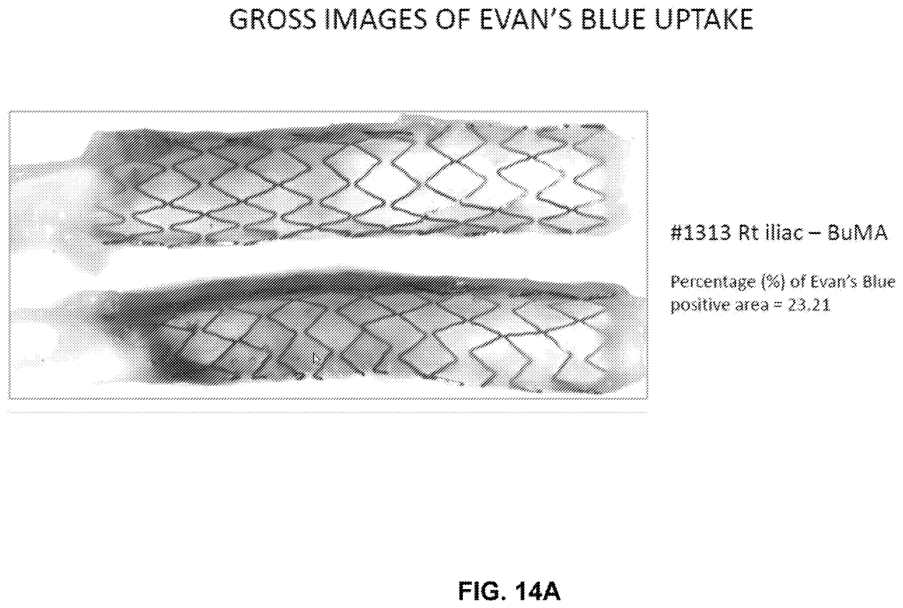

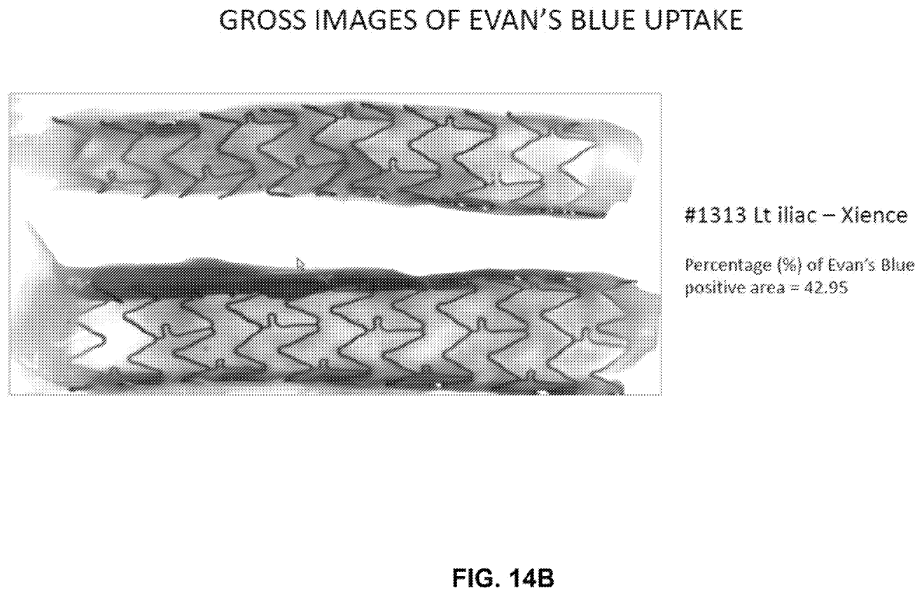

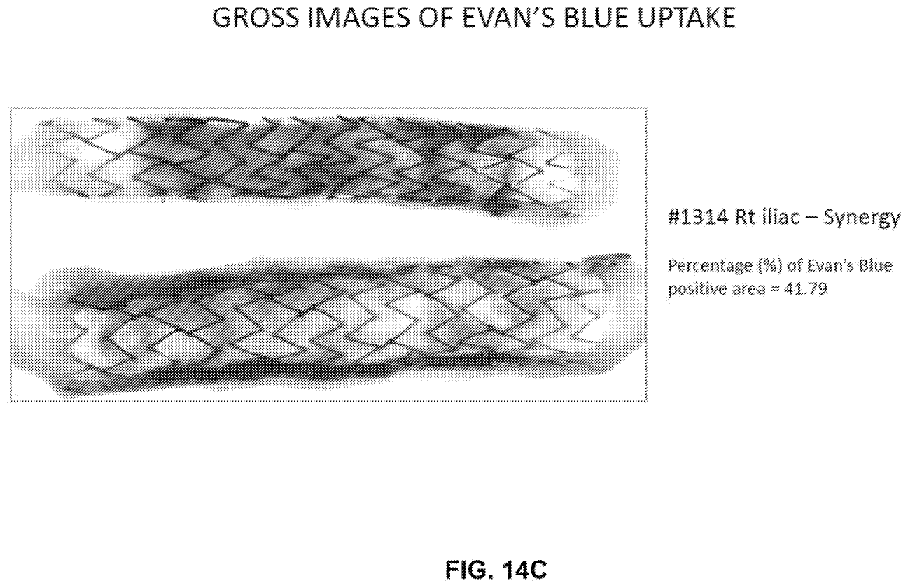

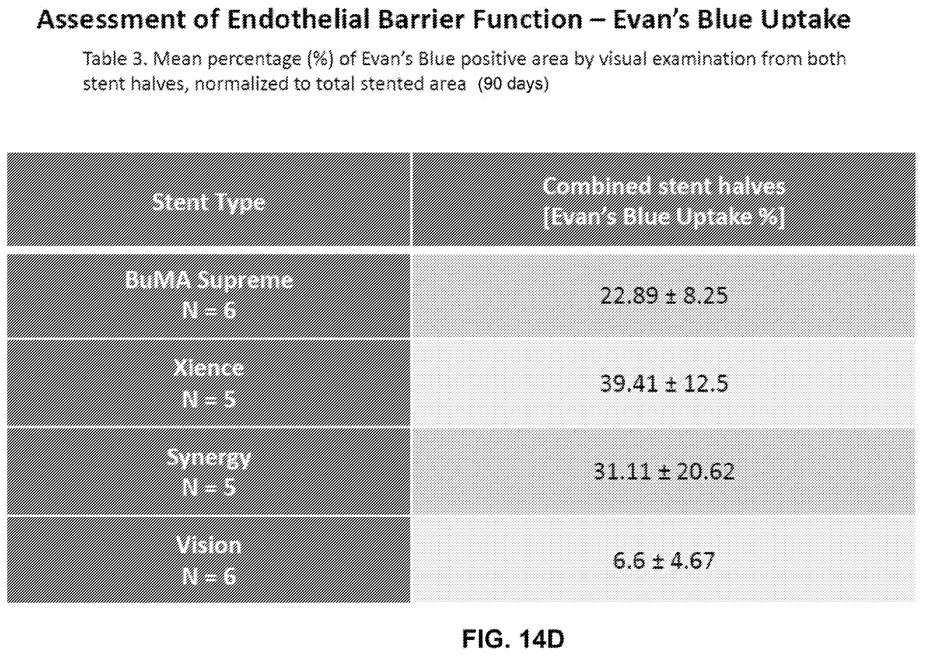

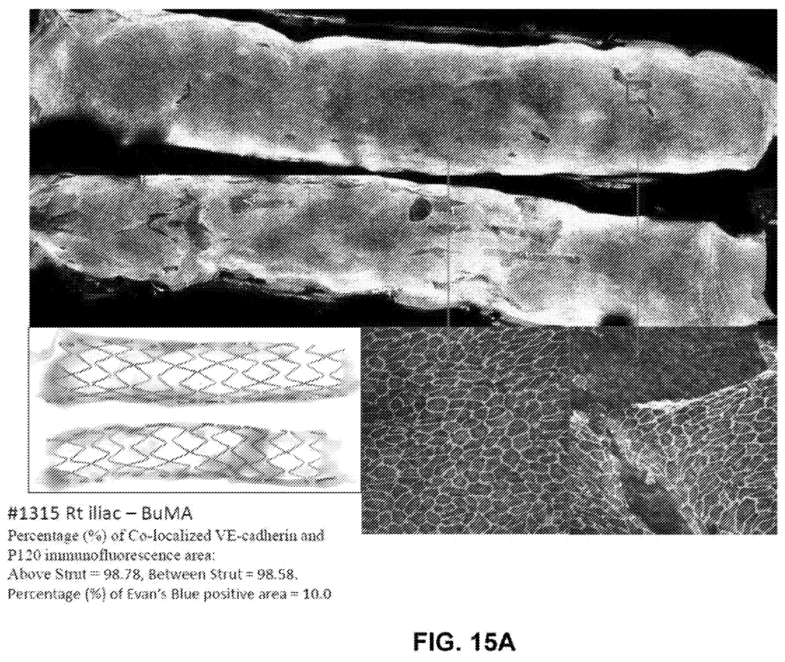

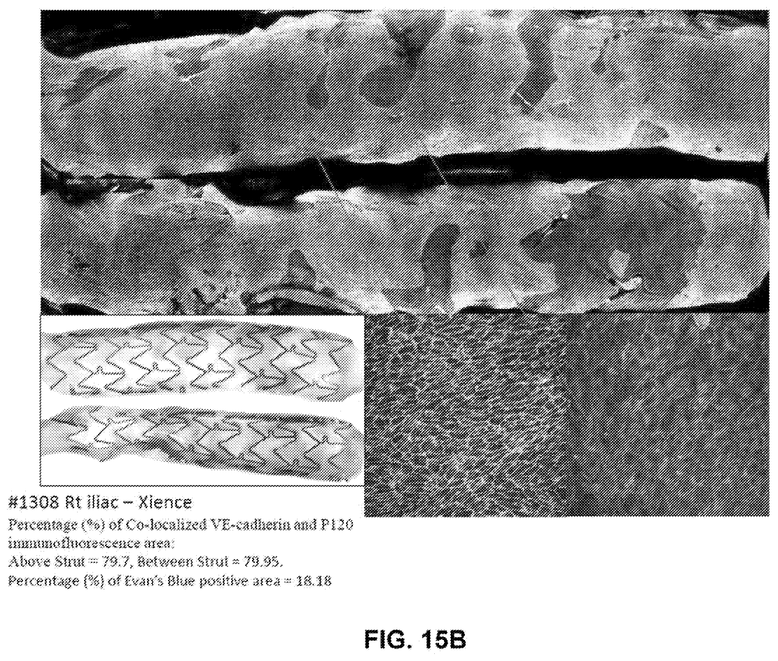

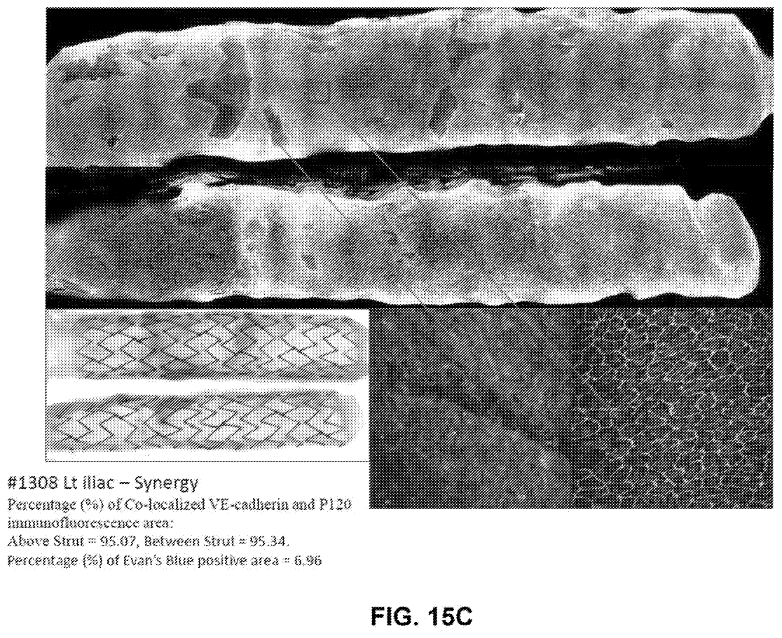

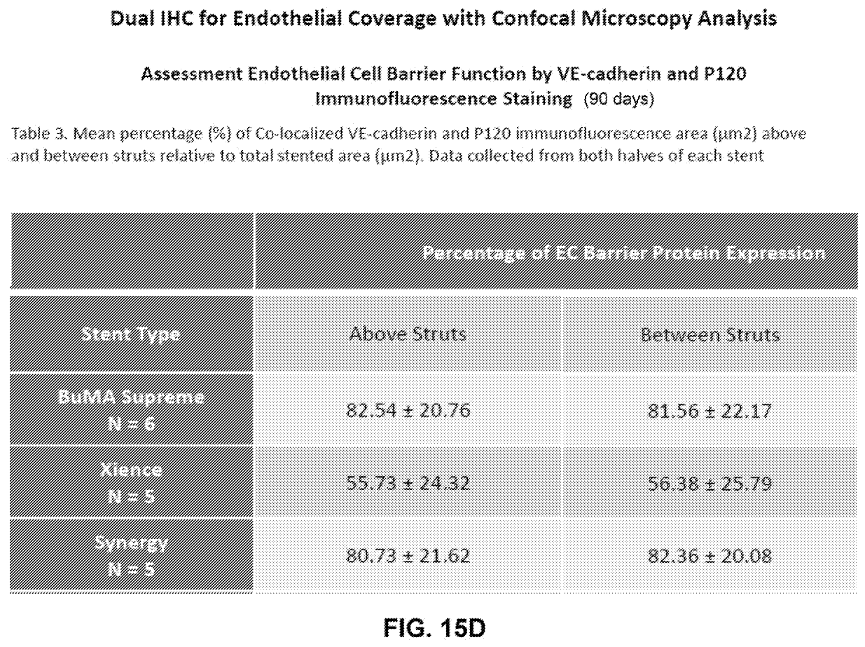

Similarly to the previous experiments, the stents were implanted into rabbits and their endothelialization was studied over time (e.g., 45 and 90 days) using Evan's Blue and VE-Cadherin/P120 colocalization. The results are exemplified in FIGS. 12A through 12D for 45 days Evan's Blue; FIGS. 13A through 13D for VE-Cadhering/P120 colocalization at 45 days; FIGS. 14A through 14D for 90 days Evan's Blue; and FIGS. 5A through 15D for VE-Cadhering/P120 colocalization at 90 days. As shown in these figures, stents according to the disclosure (BuMA Supreme stents) have a larger percentage of endothelial cell colocalization of VE-Cadhering/P120 (i.e., the endothelium is better and more functional) than other drug eluting stents tested not according to the disclosure. In addition, the permissibility of the endothelial cell layer covering the stents of the disclosure (BuMA Supreme stents), as assessed by Evan's Blue staining, is lower than that of other tested drug eluting stents not according to the disclosure, indicating that the endothelium is more functional in the BuMA Supreme stents.

It is also envisioned that the stent framework may comprise a wave design with an alternating pattern of two-three-two-three link poles spirally arranged in the axial direction. This design may improve bendability of the stent and may result in better fitting to the vessel after stent expansion. In some embodiments, both ends of the stent may have two link poles or three link poles in accordance with the two-three-two-three pattern. In other embodiments, both ends of the stent may have four link poles, which may increase axial strength of the stent. Dimensions of this example design may include, for example, a pole width of 90 μm, and a crown width of 100 μm. In having a crown width greater than the pole width, the stent may have grater radial strength and have a reduced crossing profile with the vessel after stent expansion. In addition, dimensions of this example design may include a wall thickness of 80 μm or 90 μm.

### Example 2

### Human Clinical Trials

Human clinical trials were performed with stents made of stainless steel (316L) (BUMA stents). The stents were designed to either have an OD: 1.6 and 6 crests (first design) or an OD of 1.8 and 9 crests (second design). The pole width of the first design was 110 μm and of the second design 90 μm. The wall thickness of the first design was 100 μm and of the second design was 110 μm. These stents were coated by the spray coating method described above.

A clinical trial titled “A prospective randomized controlled 3 and 12 months OCT study to evaluate the endothelial healing between a novel sirolimus eluting stent BUMA and an everolimus eluting stent XIENCE V” was done. The BUMA stent was designed with a 30-day drug release time frame and a 60-day coating/drug-containing layer bio-degradable time frame and fabricated according to Example 1 above. On the other hand, a Xience V stent is designed with a 120-day drug release time frame, and the coating is bio-stable. Twenty patients were enrolled into the study. The BUMA and XIENCE V stents were overlapped implanted at the same lesion in the same vessel of the same patient. The study showed that the struts of both stents were well-covered at 3 months and 12 months OCT follow-up. However, the struts of the BUMA stent had significantly coverage compared to the struts of the XIENCE V stent at 12 months (99.2% BUMA vs. 98.2% XIENCE V with P<0.001). Moreover, the struts of the BUMA stent had a thicker neointimal hyperplasia thickness and larger neointimal area than the struts of the XIENCE V stent (0.15±0.10 mm BUMA vs. 0.12±0.56 mm XIENCE V with P<0.001). As explained above, a thickness below a first threshold (e.g., 0.1 mm) may be indicative of an insufficient number of endothelial cells while a thickness above a second, higher threshold (e.g., 0.20 mm) may be indicative of the over proliferation of smooth cells, and endothelial cells layer is not fully functional. In addition, the BUMA stent had a more uniform strut coverage compared to the XIENCE V stent. The study shows that the BUMA stent may have better long-term safety than the XIENCE V stent.

Another clinical trial named “Biodegradable Polymer-Based Sirolimus-Eluting Stents With Differing Elution and Absorption Kinetics” was done. The BUMA stent was designed with a 30-day drug release time frame and a 60-day coating bio-degradable (disappearance/dissolution/dissipation of the drug-containing layer) time frame and fabricated according to Example 1 above. The EXCEL stent was designed with a 180-day drug release time frame and a 180-to-270-day coating bio-degradable time frame. Two thousand three hundred forty-eight patients were enrolled into the study. The BUMA stent exhibited a lower incidence of stent thrombosis than the EXCEL stent. In particular, the 1-year rate of stent thrombosis was lower with the BUMA stent than the EXCEL stent, a difference that was evidenced within the first month after implantation.

Another clinical trial named “PIONEER-II Study” compared 1-month optical coherence tomography (OCT) results between a BUMA Supreme stent and a Xience V stent. The BUMA Supreme stent was designed with a 30-day drug release time frame and a 60-day bio-degradable time frame for the drug containing layer and fabricated according to Example 1 above. The Xience V stent was designed with a 120-day drug release time frame, and the coating was bio-stable. Fifteen patients were enrolled into the study. The study showed that struts neointimal coverage at 1-month by OCT follow-up for the BUMA Supreme stent exhibited better coverage compared to the Xience V stent (83.8% BUMA vs. 73.0%, Xience V with P<0.001).

### Example 3

### Dispense Coating Process

### A. Process

In some embodiments, the drug-containing layer (3) may be formed using a dispense coating process to dispose a polymer coating on the stent framework (or on a polymer-coated stent, e.g., a stent coated in the electro-grated coating described below). In one example, after drying, a 20 millimeter stent was dispense coated with biodegradable polyester (polylactide-co-glycolide, p-PLGA) containing Sirolimus. The copolymer (5% w/v) was dissolved in chloroform. Sirolimus was then dissolved in the chloroform/polymer mixture to obtain a final ratio 1:5 Sirolimus/polymer of (1/5). A micro dispenser was run along with the stent struts and links and dispensed the mixture onto the abluminal side (8) of the stent by a micro dispenser using the following parameters:

Dispenser parameter

| | |
|---|---|
| Dispensing flow (μL/s) | 10 |
| Dispensing volume (μL/s) | 145 |
| Pressure (bar) | 0.1 |
| Dispenser run speed (mm/s) | 0.5 |
| Dispenser/stent distance (mm) | 1.1 |
| Number of dispenser run | 10 |

The coating was applied to the abluminal side (8) of the stent only. Drying at 40° C. was performed in a vacuum oven. In this example, the coating on the stent weighs 500±50 μg, and the coating thickness was about 9-12 μm. Moreover, in this example, the drug loading was 125±12 μg.

### Example 4

### Drug Release Profile for BUMA Supreme Biodegradable Drug Coated Coronary Stent System

The BuMA Supreme™ Biodegradable Drug Coated Coronary Stent System (BuMA DES) consists of a drug-coated balloon expandable stent and a rapid exchange delivery system. The cobalt chromium (CoCr) stent is coated with a very thin, non-erodible poly n-butyl methacrylate (PBMA) covalently bonded to the metal surface. A topcoat is then applied that contains sirolimus, the active ingredient, embedded in a biodegradable polymer, poly lactide co-glycolic acid (PLGA).

Release of drug from the stent and local, regional, and systemic delivery of sirolimus/rapamycin was quantified by measuring the drug content in stented coronary artery segments. Drug remaining on the stent and found in tissue was measured at 1, 3, 7, 14, 21, 28, 0, 90, and 150 day time points. Stents were implanted into Yucatan Miniature Swine pigs. The porcine and human arteries have correlatively similar anatomy and the porcine model is recommended for use in preclinical studies by the FDA, EMEA and Schwartz et al. Thirty-one Yucatan mini-swine were implanted in two of three coronary arteries: the left circumflex artery [LCx], left anterior descending artery [LAD] and right coronary artery [RCA] of each animal, when possible.

The maximum concentration of the drug in the artery (stented area) occurred at 21 days after implantation with an average of 9.85±4.72 μg/gm. Table 1. By 60 days this level had fallen to 0.92±0.20 μg/gm and at 154 and 202 days only five of the twelve samples had measurable drug levels. The drug concentration in the artery segments proximal and distal to the stented segment was much lower relative to the stented area (approximately 3-11% of the stented segment value).

TABLE 1
Quantification of Rapamycin in Arterial Tissue

| Study day | Arterial drug concentration (μg/g) |
|---|---|
| 1 | 1.63 ± 0.239 |
| 3 | 2.27 ± 0.735 |
| 7 | 2.58 ± 1.43 |
| 14 | 3.70 ± 1.71 |
| 21 | 9.85 ± 4.72 |
| 28 | 6.78 ± 4.14 |
| 60 | 0.922 ± 0.203 |
| 90 | 0.451 ± 0.188 |
| 150 | 0.109 ± 0.0121 |

The pharmacokinetic profile for the distribution of rapamycin in the arterial tissue is shown in FIGS. 16A, 16B, and 16C.

Pharmacokinetic analysis was performed for both Rabbit-PK (2014-002) and Porcine-PK (1792-318G) studies, using whole blood, target tissue, and non-target tissue concentration versus time data, using Phoenix WinNonlin (version 8.1) non-compartment analysis function (linear trapezoidal rule for AUC calculations). Nominal dose values and sampling times were used for calculations. Any concentration reported as BLQ (LLOQ<0.100 ng/mL) was set equal to zero. Time points were manually evaluated for the determination of lambda z (R₂>0.75), the elimination rate constant, upon which half-life (t_{1/2}) was based. Half-life was calculated using whole blood data through 24 hours (rabbit) or 72 hours (porcine) post implantation as the majority of the clearance appeared to occur during this duration.

For the rabbit and porcine study, pharmacokinetic analyses included Cmax, Tmax, Tlast, AUC0-24, AUClast, t1/2, and MRTlast and is presented in Table 2. Below.

TABLE 2
Pharmacokinetic Bioanalysis

| | | | | | | | | |
|---|---|---|---|---|---|---|---|---|
| | DOSE | C_{MAX} | T_{MAX} | AUC₀₋₂₄ | AUC_{LAST} | T_{LAST} | t_{1/2} | MRT |
| STATISTICS | (μg) | (ng/ml) | (hr) | (hr*ng/ml) | (hr*ng/ml) | (hr) | (hr) | (hr) |

RABBIT STUDY (DEVICE SIZE = 3.00 × 15 mm)
BLOOD

| | | | | | | | | | |
|---|---|---|---|---|---|---|---|---|---|
| N | 89 | μg | 4 | 4 | 4 | 4 | 4 | 3 | 4 |

| | | | | | | | |
|---|---|---|---|---|---|---|---|
| MEAN | 14.5 | 0.083 | 88.9 | 237 | 120 | 8.80 | 45.6 |
| SD | 0.569 | 0.0 | 19.1 | 80.2 | 55 | 0.902 | 22.3 |
| CV % | 3.9 | 0.0 | 21.5 | 33.9 | 46.2 | 10.3 | 48.8 |

TARGET TISSUE

| | | | | | | | | | |
|---|---|---|---|---|---|---|---|---|---|
| ARTERY (L) | 89 | μg | 2300 | 670 | — | 1770000 | 1080 | NC | — |

| | | | | | | | |
|---|---|---|---|---|---|---|---|
| ARTERY(R) | 3790 | 670 | — | 2470000 | 1080 | NC | — |
| KIDNEY (L) | 42.0 | 170 | — | 17500 | 672 | NC | — |
| KIDNEY (R) | 52.7 | 72 | — | 17100 | 672 | 283 | — |
| LIVER | 21.6 | 24 | — | 5680 | 672 | 295 | — |
| LUNG | 9.85 | 72 | — | 3530 | 672 | 290 | — |
| MYOCARDIUM | 13.1 | 24 | — | 4650 | 672 | 244 | — |
| SPLEEN | 58.4 | 170 | — | 24500 | 672 | NC | — |
| STENT (L) | — | — | — | — | — | 102 | — |
| STENT (R) | — | — | — | — | — | 110 | — |

PORCINE STUDY (DEVICE SIZE = 3.00 × 15 mm AND 3.50 × 15 mm)
BLOOD

| | | | | | | | | | |
|---|---|---|---|---|---|---|---|---|---|
| N | 89 | μg | 6 | 6 | 6 | 6 | 6 | 4* | 6 |
| MEAN | 121 | μg | 1.59 | 0.72 | 20.3 | 50.3 | 464 | 10.4 | 217 |

| | | | | | | | |
|---|---|---|---|---|---|---|---|
| SD | 0.206 | 1.1 | 2.57 | 25.1 | 323 | 5.66 | 164 |
| CV % | 12.9 | 155.6 | 12.7 | 50.0 | 69.5 | 54.4 | 75.7 |

MODULE 1
MYOCARDIUM
NOTE: THESE VALUES WERE DERIVED FROM THE
TISSUE CONCENTRATIONS (ng/g) AS OBSERVED ON
DAYS 1, 3, 7, 14, 21, 28, 60, AND 90.
THESE VALUES ARE PROVIDED FOR INFORMATIONAL
PURPOSES ONLY AND ARE TO BE INTERPRETED WITH CAUTION.

| | | | | | | | | | |
|---|---|---|---|---|---|---|---|---|---|
| LAD (BENEATH) | 89 | μg | 103 | 24 | — | 67700 | — | — | — |
| LAD (DOWNSTREAM) | 121 | μg | 20.1 | 340 | — | 6300 | — | — | — |

| | | | | | | | |
|---|---|---|---|---|---|---|---|
| RCA (BENEATH) | 174 | 500 | — | 73200 | — | — | — |
| RCA (DOWNSTREAM) | 17.4 | 500 | — | 9390 | — | — | — |

ARTERY TISSUE
NOTE:
THESE VALUES WERE DERIVED FROM THE
TISSUE CONCENTRATIONS (mg/g) AS OBSERVED ON
DAYS 1, 3, 7, 14, 21, 28, 60, 90, 150, 200.
THESE VALUES ARE PROVIDED FOR INFORMATIONAL
PURPOSES ONLY AND ARE TO BE INTERPRETED WITH CAUTION.

| | | | | | | | | |
|---|---|---|---|---|---|---|---|---|
| LAD DISTAL UNTREATED | 89 | μg | 0.165 | 500 | 80.4 | — | — | — |
| LAD TREATED | 121 | μg | 11.0 | 500 | 6580 | — | — | — |

| | | | | | | |
|---|---|---|---|---|---|---|
| LCX TREATED | 3.29 | 340 | 3520 | — | — | — |
| RCA DISTAL UNTREATED | 0.438 | 170 | 233 | — | — | — |
| RCA PROXIMAL UNTREATED | 1.29 | 170 | 186 | — | — | — |
| RCA TREATED | 9.23 | 670 | 8240 | — | — | — |

*ANIMALS OE-3 AND OE-6 DID NOT EXHIBIT A WELL CHARACTERIZED TERMINAL ELIMINATION PHASE RESULTING IN AN INABILITY TO CALCULATE t_{1/2}.
TISSUE PARAMETERS ARE REPORTED IN UNITS OF ng/g FOR C_{MAX} AND hr*ng/g FOR AUC.
N/A = NOT APPLICABLE, NC = NOT CALCULATED DUE TO LIMITED TERMINAL CONCENTRATION DATA
AUC₀₋₂₄ = AREA UNDER THE WHOLE BLOOD DRUG CONCENTRATION-TIME CURVE BETWEEN TIMES ZERO AND 24 hrs.
AUC_{LAST} = AREA UNDER THE CURVE FROM THE TIME OF DOSING TO THE TIME OF THE LAST MEASURABLE CONCENTRATION.
BLQ = BELOW LIMIT OF QUANTITATION,
C_{MAX} = THE MAXIMUM CONCENTRATION OBSERVED FOR EACH STUDY GROUP
CV % = COEFFICIENT OF VARIATION,
LLOQ = LOWER LIMIT OF QUANTITATION,
MRT_{LAST} = MEAN RESIDENCE TIME BETWEEN TIME ZERO TO THE TIME OF THE LAST,
N = NUMBER OF OBSERVATIONS,
NA = NOT APPLICABLE,
PK = PHARMACOKINETIC,
SD = STANDARD DEVIATION,
t_{1/2} = FIRST ORDER TERMINAL ELIMINATION HALF-LIFE,
t_{LAST} = THE TIME OF LAST QUANTIFIABLE SAMPLE CONCENTRATION,
t_{MAX} = THE TIME AT WHICH C_{MAX} OCCURRED

### Example 5

BP-DES, BuMA Supreme® (SINOMED, Tianjin, China) sirolimus-eluting stent (BP-SES) is characterized by metallic stent composed of cobalt chromium circumferentially covered with an ultra-thin layer of poly-butyl methacrylate over which is attached a top-coat biodegradable layer of poly lactic-co-glycolic acid (PLGA), that acts as a sirolimus reservoir. This stent design has the shortest polymer degradation/drug release profile of the currently available BP-DESs occurring within 6 weeks. Tissue drug levels in this BP-DES remain present for a shorter duration compared with DP-DESs where therapeutic drug levels persist well beyond 90 days.

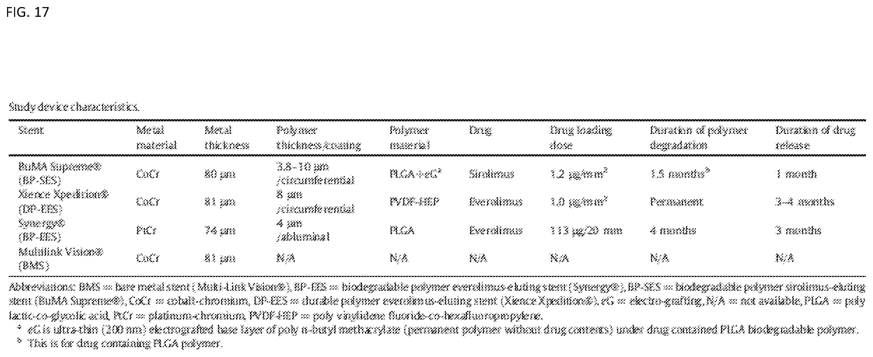

In this series of studies, the performance of DESs including BP-SES, DP everolimus-eluting stent (DP-EES, Xience Xpedition®, Abbott Vascular, California, USA), BP everolimus-eluting stent (BP-EES, Synergy®, Boston Scientific, Massachusetts, USA), and BMS (Multi-Link Vision®, Abbott Vascular, California, USA) were assessed. The relative differences between these stent designs are listed in FIG. 17. BP-SES is superior to the other stents (e.g., it has improved safety).

### Endothelial Functional Healing in Rabbit Ilio-Femoral Arteries

Following balloon denudation, drug-eluting stents were implanted to both right and left ilio-femoral arteries. Rabbits were injected with Evans blue dye(EBD) administered i.v. at 45 or 90 days under general anesthesia. EBD was allowed to re-circulate for 1 h to assess vascular permeability. Intravenously administered EBD results in the spontaneous binding to serum albumin, and subjection of the arterial wall to the 70 kD large complex. Blue staining of the arterial wall indicates disruption of the endothelial luminal barrier. Following euthanasia, the stented iliac arteries were dissected and removed. The EBD positive area was estimated per strut column and averaged for the entire stent. To compare endothelial barrier integrity, each stent half was stained with anti-VE-cad and p120 antibodies, and fluorescent images were obtained under confocal microscopy. Areas with competent endothelial barrier formation were defined as areas in which p120/VE-cad co-localized at cell borders as previously reported and were analyzed for each stent. Cell height and width of endothelial cells which demonstrated co-localization of p120/VE-cad were also assessed to obtain cell shape index (cell height divided by cell width) which represents endothelial cell morphology. In order to compare diagnostic ability of EBD and p120/VE-cad colocalization, spatial distribution of the area with endothelial dysfunction assessed by these two different methods were further analyzed. Following confocal microscopic analysis, the stented artery halves underwent scanning electron microscopy (SEM) analysis. Endothelial tissue coverage of the stented site was assessed by semi-quantification, visual estimation from the proximal to distal end. Stents were excluded from the study if there was evidence of damage to endothelial layer upon examination by SEM.

New Zealand White rabbits were randomized to receive BP-SES, DPEES, BP-EES, or BMS in their iliac arteries. All rabbits (n=24) survived the in-life portion of the study and all stents at termination were patent without any stent fracture assessed by angiography. Quantitative vessel angiography analysis revealed that followup diameter of stented site was significantly lower and percent late lumen loss was significantly greater in BMS compared with all three DESs at both 45- and 90-day cohorts. BP-SES showed comparable results in terms of follow-up diameter as well as late lumen loss versus DP-EES and BP-EES at both 45- and 90-day.

### Assessment of Endothelial Permeability by Evans Blue Dye Staining at 45 and 90 Days

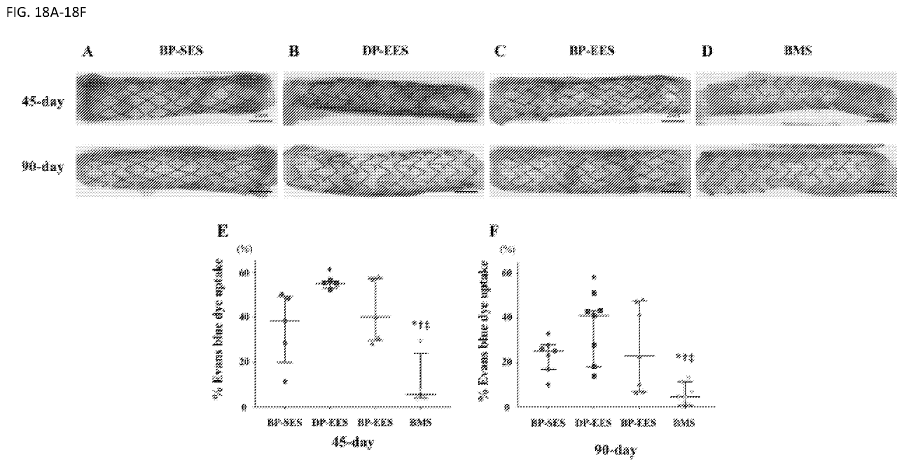

FIG. 18 shows representative whole stent images of EBD at 45 and 90 days in each group (BP-SES, DP-EES, BP-EES and BMS). EBD uptake after 45-day implantation was least for BMS (5.8%), followed by BPSES (38.4%), BP-EES (40.2%), and DP-EES (55.1%) (FIG. 18E) BMS was statistically superior (i.e. had the least uptake) regarding of EBD uptake versus all DES. Among the three DES, post-hoc analysis of EBD uptake at 45 days showed statistical significance between BP-SES vs DP-EES (P=0.01) with no differences between BPSES and BP-EES. Overall, maturation and barrier function of luminal endothelium was relatively greater for all stents in animals at 90 days compared with 45 days as expected. At the 90-day time point, EBD uptake remained least for BMS (4.4%), which was less than BP-SES (25.2%), BP-EES (22.9%) and DP-EES (41.1%) (FIG. 18F) BMS remained statistically significant versus all DES and BP-SES showed lower EBD uptake than that of DP-EES (P=0.03) but was not different from BP-EES.

### Assessment of Endothelial Barrier Protein Expression and Cell Morphology

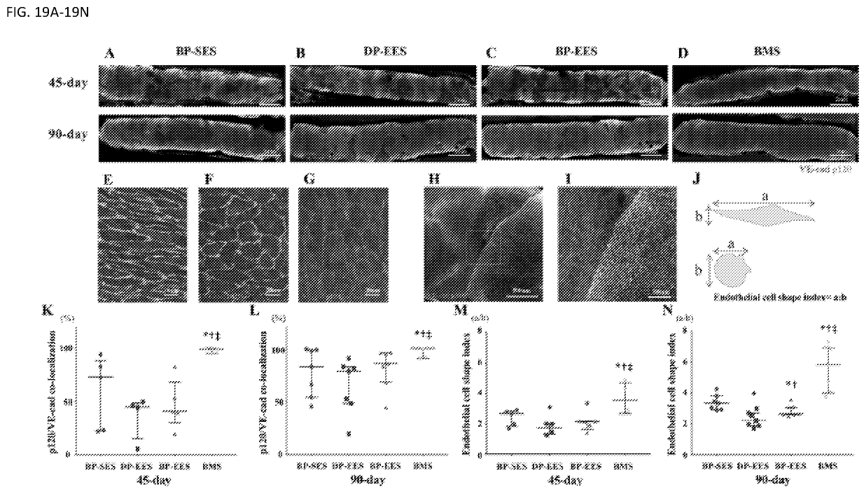

FIG. 19A-D shows representative images of p120/VE-cad doublestaining for each type of stent at 45 and 90 days. Mean percentage of co-localized p120/VE-cad area relative to total stented area was obtained from stented segments. Endothelial cells which show colocalization of p120/VE-cad at cell borders (FIG. 19E-F) indicate proper alignment of these two molecules consistent with proper barrier function. Whereas, cells without p120/VE-cad co-localization (FIG. 19G) indicate improper alignment of these two molecules consistent with poor barrier function. The boundary between these two types of cell areas was obvious on low (FIG. 19H) and medium power (FIG. 191) confocal images. At 45 days, supportive of the EBD data, expression of co-localizedp120/VE-cad was relatively weak in all DES and the expression of p120/VE-cad was statistically greater for the BMS vs all three DESs. Overall, percent area coverage for p120/VE-cad was greatest in BMS(99.5%). Conversely, DESs showed lower percent area of p120/VE-cad (BP-SES=79.1%, DP-EES=45.5%, and BP-EES=36.3%) with no statistical differences between the three (FIG. 19K). Consistent with the 45-day data, 90-day data revealed that BMS maintained significantly higher percent area of p120/VE-cad co-localization above struts (100.0%) versus all DESs (BP-SES=84.0%, DP-EES=79.1%, and BP-EES=84.9%) while no statistical differences were noted among DESs (FIG. 19L).

In cells that expressed p120/VE-cad complex around their membrane border, their cell shape appeared different among the 4 types of stents. FIG. 19E-F represent spindle and cobble-stone like endothelial cells, respectively. To reveal these differences among stent types, we applied the endothelial cell shape index (the cell height divided by width) (FIG. 19J) as described previously [Mori H, Cheng Q, Lutter C, Smith S, Guo L, KutynaM, et al. Endothelial barrier protein expression in biodegradable polymer sirolimus-eluting versus durable polymer Everolimus-eluting metallic stents. JACC Cardiovasc Interv. 2017; 10:2375-87.]At 45 days, cell shape index was higher in BMS (3.57), followed by BP-SES (2.69), BP-EES (2.14) and DP-EES (1.73) (FIG. 19M). Significant differences were observed not only between BMS and all DESs but in BP-SES vs DP-EES (P<0.01) and BP-EES (P=0.02). Although the index was relatively greater for all stents at 90 days versus 45 days, these differences among stents were still evident at the 90-day timepoint (BMS=5.83, BP-SES=3.34, BP-EES=2.65 and DP-EES=2.20) (FIG. 19N) Statistical significance was observed between BMS vs three DESs (all P<0.01). Additionally, differences were significant between BP-SES vs BP-EES (P=0.02) and DP-EES (P<0.01), and BP-EESvs DP-EES (P<0.01). The spindle shape itself may indicate the healthy functional state of endothelial cells which originates from normal adherens junction. The confocal microscopic findings revealed significantly greater cell shape index in the order of BMS>BP-SES>BP-EES>DP-EES at the area of p120/VE-cadc o-localization both in 45- and 90-day time points. This finding may also support the concept that the drug/polymer PK profile in BP-DESs is important in determining the time course of endothelial functional recovery.

Spatial Distribution of Evans Blue Staining and p120/VE-Cadherin Colocalization Area

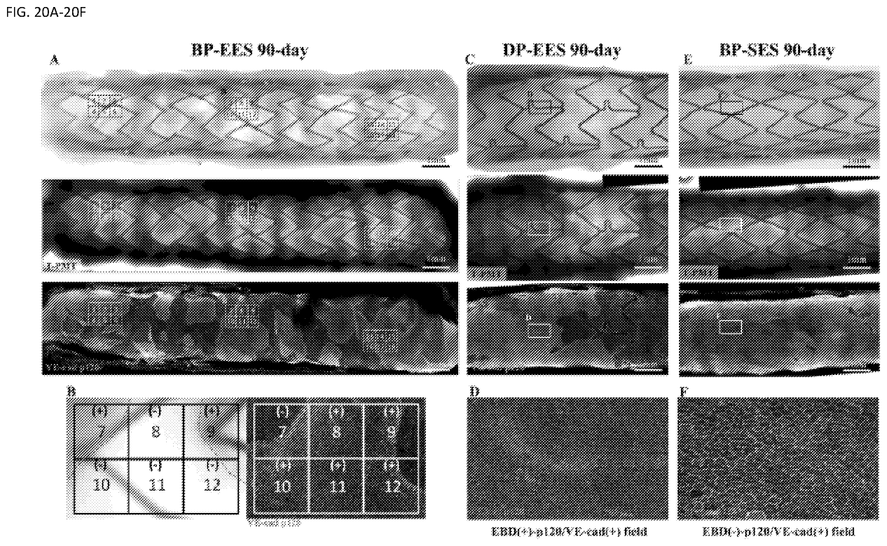

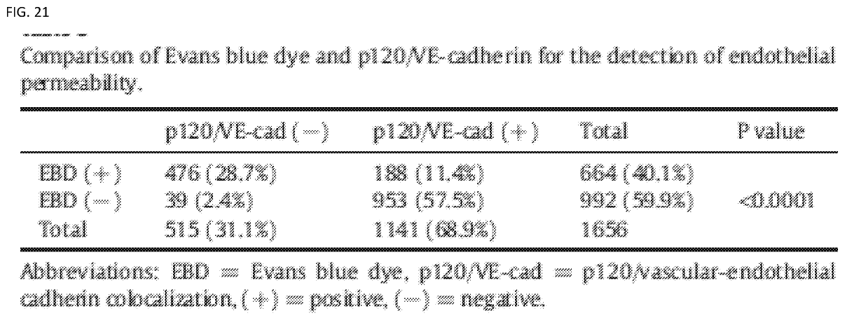

Two different methods for assessing endothelial permeability, EBD
and p120/VE-cad co-localization, were compared in terms of spatial distribution. The methodology for coregistration of images was described in FIG. 20A-B. 18 regions of interest (ROI) fields were applied to each stent half and total 36 ROI information was obtained from each stent. Finally, a total of 1656 ROIs were analyzed (648 and 1008 from 45-day and 90-day cohort, respectively). Fischer's exact test showed a significant correlation between positive EBD [EBD(+)] and negative co-localization of p120/VE-cad [p120/VE-cad(−)](P<0.0001). When the EBD(+) was considered as gold standard for Endothelial permeability, sensitivity and specificity of p120/VE-cad(−) in the diagnosis of endothelial permeability were 71.7% and 96.1%, respectively (FIG. 21).

Because the number of EBD(+)-p120/VE-cad(+) fields were not small (11.4%) in our analysis, the difference between EBD (+)-p120/VE-cad(+) and EBD (−)-p120/VE-cad(+) fields was carefully examined. FIG. 20C-F indicates the representative example for the difference between EBD(+)-p120/VE-cad(+) and EBD(−)-p120/VE-cad(+) fields. EBD(+)-p120/VE-cad(+) area (FIG. 20C-D) showed relatively lower expression of VE-cad at cell membranes compared with EBD(−)-p120/VE-cad(+) areas (FIG. 20E-F).

### Assessment of Endothelial Stent Coverage by SEM

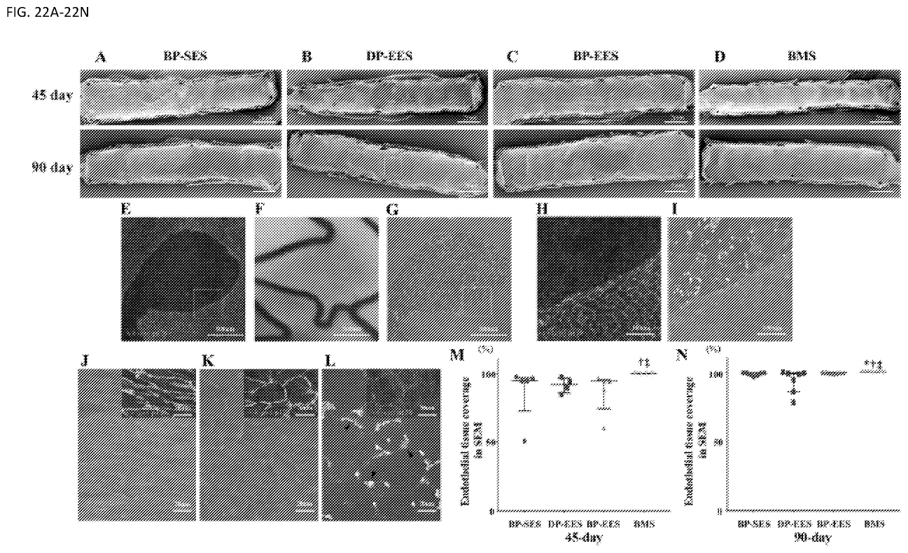

FIG. 22A-D represents SEM images in each type of stent at 45 and 90 days. Border zone between complete and incomplete p120/VE-cad co-localization could also be confirmed by SEM and EBD (FIG. 22E-I). Furthermore, spindle and cobble-stone endothelial cells with p120/VE-cad co-localization shows smooth cell surface in high power SEM images (FIG. 22J-K). To the contrary, areas which lacked p120/VE-cad colocalization revealed aggregation of platelets and leukocyte at the sites of intercellular ridges by SEM (FIG. 22L). Finally, we assessed the endothelial tissue coverage using SEM. Overall % endothelial tissue coverage of stented segment at 45 days was greatest for BMS (100.0%)<BP-SES(94.1%)<BP-EES (90.5%)<DP-EES (85.1%) (FIG. 22M). Only BMS vs DP-EES showed statistical significance. Percent endothelial tissue coverage of stented segment was greater for all stents at 90 days compared to 45 days (BMS=100.0%, BP-SES=98.8%, BPEES=99.5%, and DP-EES=96.8%). BMS vs DESs still showed statistical difference while no statistical differences were noted among DESs (FIG. 22N).

### Definitions

Units, prefixes, and symbols are denoted in their Systeme International de Unites (SI) accepted form. Numeric ranges are inclusive of the numbers defining the range. The disclosure provided herein are not limitations of the various aspects of the application, which may be by reference to the specification as a whole.

The articles “a” or “an” refer to “one or more” of any recited or enumerated component

The terms “about” or “comprising essentially of” refer to a value or composition that is within an acceptable error range for certain value or composition as determined by one of ordinary skill in the art, which will depend in part on how the value or composition is measured or determined, i.e., the limitations of the measurement system. For example, “about” or “comprising essentially of” may mean within 1 or more than 1 standard deviation per the practice in the art. Alternatively, “about” or “comprising essentially of” may mean a range of up to 10% (i.e., ±10%). For example, about 3 mg may include any number between 2.7 mg and 3.3 mg (for 10%). With respect to biological systems or processes, the terms may mean up to an order of magnitude or up to 5-fold of a value. When certain values or compositions are provided in the application and claims, unless otherwise stated, the meaning of “about” or “comprising essentially of” include an acceptable error range for that value or composition. Any concentration range, date range, percentage range, ratio range, or integer range includes the value of any integer within the recited range and, when appropriate, fractions thereof (such as one-tenth and one-hundredth of an integer), unless otherwise indicated.

The term “and/or” refer to each of the two specified features or components with or without the other. Thus, the term “and/or” as used in a phrase such as “A and/or B” herein is intended to include “A and B,” “A or B,” “A” (alone), and “B” (alone). Similarly, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “e.g.,” and “i.e.” are used merely by way of example, without limitation intended, and not be construed as referring only those items explicitly enumerated in the specification.

The terms “or more”, “at least”, “more than”, and the like, e.g., “at least one” include but not be limited to at least 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more than the stated value. Also included is any greater number or fraction in between. The term “no more than” includes the stated value and each value less than the stated value. For example, “no more than 100 micrometers” includes 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, micrometers/days/%, etc. Also included is any lesser number or fraction in between.

Throughout the specification the word “comprising,” or variations such as “comprises” or “comprising,” is understood to imply the inclusion of a stated element, integer or step, or group of elements, integers or steps, but not the exclusion of any other element, integer or step, or group of elements, integers or steps. It is understood that wherever aspects are described herein with the language “comprising,” otherwise analogous aspects described in terms of “consisting of” and/or “consisting essentially of” are also provided.

A “therapeutically effective amount,” “therapeutically effective dosage,” or the like refers to an amount of the cells (such as immune cells or engineered T cells) that are produced by the present methods and that, when used alone or in combination with another therapeutic agent, protects or treats a subject against the onset of a disease or promotes disease regression as evidenced by a decrease in severity of disease symptoms, an increase in frequency and duration of disease symptom-free periods, and/or prevention of impairment or disability due to disease affliction. The ability to promote disease regression may be evaluated using a variety of methods known to the skilled practitioner, such as in subjects during clinical trials, in animal model systems predictive of efficacy in humans, or by assaying the activity of the agent in in vitro assays.

A “patient” as used herein includes any human who is afflicted with a disease or disorder, including a cardiac disease or disorder (e.g., arterial blockage). The terms “subject” and “patient” are used interchangeably herein.

The terms “reducing” and “decreasing” are used interchangeably herein and indicate any change that is less than the original. “Reducing” and “decreasing” are relative terms, requiring a comparison between pre- and post-measurements.

The use of the term “artery” or “arterial” is not limited to cardiovascular arteries. The stents of the disclosure can be used in the manufacture of devices for treating or preventing any vascular disease in any organ (heart, brain, lung, kidney, etc), including angiostenosis or to prevent restenosis, thrombosis, tumor growth, or angiomas. They can also be used for treatment of obstruction of lacrimal glands.

A covered strut is defined as a strut having a neointimal thickness above 20 micrometers (um). In some embodiments, the neointimal thickness is >20-120.0 um; e.g., 120.1-160.0 um. In a preferred embodiment, the neointimal thickness is between 20 and 160, preferably between 20 and 150 um at 2 months, in a rabbit iliac artery model. In some embodiments, the preferred neointima thickness in humans (measured by OCT) is between 20 and 80 um at 3 months, and preferably between 140 and 160 um at 12 months post-stent implantation.

The term “80-90% neointima coverage over the stent struts” means that 80-90% of the global surface area of the whole stent is covered by a neointima having a thickness above zero micrometers, preferably, 20 micrometers or greater.

The term neointimal thickness may be defined as in Takano M, Inami S, Jang I K, Yamamoto M, Murakami D, Seimiya K, Ohba T, Mizuno K. Evaluation by optical coherence tomography of neointimal coverage of sirolimus-eluting stent three months after implantation. Am J Cardiol. 2007; 99:1033-8.

The described embodiments are to be considered in all respects only as illustrative and not as restrictive. The scope of the present disclosure is, therefore, indicated by the appended claims rather than by the foregoing description. All changes which come within the meaning and range of the equivalence of the claims are to be embraced within their scope.

1. A drug eluting stent, comprising at least four parts:
a stent framework;
a drug-containing layer;
a drug embedded in the drug-containing layer; and
a biocompatible base layer disposed over the stent framework and supporting the drug-containing layer,
wherein one or more parts of the stent are designed to achieve a pre-designed drug release pharmacokinetic profile selected from:
(1) the drug pharmacokinetic profile has Tmax and Cmax (expressed in μg of drug per g of the artery tissue after implantation) so that:
(a) Tmax is between 400 and 600 hr, preferably 500 hr,
(b) Cmax is between 5 and 15 μg/g, preferably 10 μg/g, and/or
(c) the drug pharmacokinetic profile overlaps with the kinetic profile for smooth muscle cell proliferation post-stent implantation, as depicted in FIG. 16B, preferably wherein the arterial tissue concentration at the site of stent implantation peaks between 15 and 25 days, preferably at 20 days, and then decreases to allow for vascular restoration; and
(2) the drug has a pharmacokinetic profile in the arterial tissue at the site of stent implantation about as depicted in FIG. 16A or FIG. 16B, optionally, wherein the drug is sirolimus.

2. The drug eluting stent of claim 1, wherein the drug is embedded essentially on the drug-containing layer on an abluminal side of the stent.

3. The drug eluting stent of claim 1, wherein:
(i) the stent framework is fabricated from a single piece of metal, wire, or tubing; preferably, wherein the metal comprises at least one of stainless steel, nitinol, tantalum, cobalt-chromium MP35N or MP20N alloys, platinum, and titanium; or a biodegradable material, such as a metallic alloy made from magnesium, zinc or iron;
(ii) the drug comprises at least one of an antithrombogenic agent, an anticoagulant, an antiplatelet agent, an antineoplastic agent, an antiproliferative agent, an antibiotic, an anti-inflammatory agent, a gene therapy agent, a recombinant DNA product, a recombinant RNA product, a collagen, a collagen derivative, a protein analog, a saccharide, a saccharide derivative, an inhibitor of smooth muscle cell proliferation, a promoter of endothelial cell migration, proliferation, and/or survival, and combinations of the same; and/or
(iii) the drug-containing layer is selected from the group consisting of poly(hydroxyalkanoates) (PHAs), poly(ester amides) (PEAs), poly(hydroxyalkanoate-co-ester amides), polyacrylates, polymethacrylates, polycaprolactones, poly(ethylene glycol)(PEG), poly(propylene glycol)(PPG), poly(propylene oxide) (PPO), poly(propylene fumarate) (PPF), poly(D-lactide), poly(L-lactide), poly(D,L-lactide), poly(meso-lactide), poly(L-lactide-co-meso-lactide), poly(D-lactide-co-meso-lactide), poly(D,L-lactide-co-meso-lactide), poly(D,L-lactide-co-PEG), poly(D,L-lactide-co-trimethylene carbonate), poly(lactide-co-glycolide), poly(glycolic acid-co-trimethylene carbonate), poly(trimethylene carbonate), PHA-PEG, PBT-PEG (PolyActive®), PEG-PPO-PEG (Pluronic®), and PPF-co-PEG, polycaprolactones, polyglycerol sebacate, polycarbonates, biopolyesters, polyethylene oxide, polybutylene terephalate, polydioxanones, hybrids, composites, collagen matrices with grouth modulators, proteoglycans, glycosaminoglycans, vacuum formed small intestinal submucosa, fibers, chitin, dexran, and mixtures thereof; preferably, wherein the drug-containing layer is selected from tyrosine derived polycarbonates, poly(β-hydroxyalcanoate)s and derivatives thereof, or comprises a polylactide-co-glycolide 50/50 (PLGA) or Poly Butyl MethAcrylate.

4.-6. (canceled)

7. The drug eluting stent of claim 3, wherein the drug comprises sirolimus and/or a derivative or analog of sirolimus.

8. The drug eluting stent of claim 1, wherein the drug-containing layer has a thickness between 5 and 12 μm or 2-20 μm, preferably 1, 2, 3, 4, 5, 6, 7, 8, 9, 10, 11, 12, 13, 14, 15, 16. 17, 18, 19, or 20 μm in either the luminal, abluminal, or both sides.

9.-12. (canceled)

13. The drug eluting stent of claim 1, wherein the biocompatible base layer comprises Li) at least one of poly poly-butyl methacrylate, poly-N-[Tris(hydroxymethyl)-methyl]-acrylamide (poly-NTMA), Poly-dopamine, PEDOT, PTFE, PVDF-HFP, poly(styrene-b-isobutylene-b-styrene), Parylene C, PVP, PEVA, SBS, PC, TiO2; and (ii) an electro-grafted layer, optionally an electro-grafted polymeric layer, optionally interdigitating with the drug-containing layer, preferably, wherein the electro-grafted polymeric layer comprises a monomer selected from the group consisting of vinylics, epoxides, and cyclic monomers undergoing ring opening polymerization and aryl diazonium salts.

14. (canceled)

15. The drug eluting stent of claim 1, wherein the biocompatible base layer comprises an organic layer obtained by chemical grafting of phenyl diazoniums or azides.

16. The drug eluting stent of claim 13, wherein the grafted layer has a thickness between 10 nm and 1000 nm, preferably between 100 nm and 200 nm.

17. (canceled)

18. The drug eluting stent of claim 13, wherein the monomer is further selected from the group consisting of butyl methacrylate, methyl methacrylate, hydroxyethyl methacrylate, epsilon caprolactone, N-[Tris(hydroxymethyl)-methyl]-acrylamide (NTMA) and 4-nitrophenyl diazonium tetrafluoro borate.

19. A method of (i) selecting the product parameters of a drug eluting stent and/or (ii) predicting the outcome of the stent implantation at 1-year or more post-stent implantation (e.g., thrombosis), comprising preparing the stent and measuring the percentage of neointima coverage over the stent in the arterial tissue where a stent is implanted at 30 days post-stent implantation, wherein the higher the percentage of neointima coverage over the stent at 30 days, the better the stent in terms of stent efficacy and/or safety; preferably, wherein the percentage of neointima coverage over the implanted stent at about 30 days/i month is predictive of stent implantation side effects at 1-year or more post-stent implantation, wherein a 80-90% neointima coverage at about 30 days/1 month is representative or predictive of low side effects at 1-year post-stent implantation; and/or, wherein the percentage of neointima coverage may be assessed by measuring strut coverage, preferably at about 30 days/1 month; and/or wherein a covered strut is a strut having a neointimal thickness above 0, preferably above 20, micrometers above the surface of the strut.

20.-23. (canceled)

24. A method of preparing a drug-eluting stent, wherein the drug-eluting stent achieves between 80% and 100% neointima strut coverage between day 28 and day 90 post-stent implantation in an animal model, and preferably a rabbit iliac artery model, comprising preparing a stent with the properties of the stent of claim 1; preferably, wherein 80%-100% neointima strut coverage is achieved between day 20 and day 60 post-stent implantation.

25. (canceled)

26. The method of claim 24, wherein 80%-100% neointima strut coverage is achieved about 30 days post-stent implantation.

27. A drug-eluting stent, comprising at least four parts:
a stent framework;
a drug-containing layer;
a drug embedded in the drug-containing layer; and
a biocompatible base layer disposed over the stent framework and supporting the drug-containing layer, wherein the stent has the following characteristics in a rabbit trial, after implantation in the iliac artery:
(a) the uptake of Evans' Blue dye by the artery in the stented zone is <40% at 45 days and <25% at 90 days;
(b) The ratio R, measured by confocal microscopy in a longitudinal cross-section of the stented zone of the stented artery, of the quantity of P120 protein to that of VE-Cadherin (R=[P120]/[VE-cad]), which characterizes the degree of co-localization of the said proteins in the scaffolded region, is higher than 70% at 45 days, and higher than 80% at 90 days; and
(c) The cell shape index I, defined as the ratio between the largest length [a] of endothelial cells observed by confocal microscopy divided by the size [b] in the direction perpendicular to said longest length (I=[a]/[b]), is larger than 2 at 45 days after implantation, and larger than 3.5 at 90 days after implantation.

28. The stent of claim 27, wherein one or more parts of the stent are designed to achieve a pre-designed drug release pharmacokinetic profile selected from:
(1) the drug pharmacokinetic profile having Tmax and Cmax (expressed in μg of drug per g of the artery tissue after implantation) so that:
(a) Tmax is between 400 and 600 hr, preferably 500 hr,
(b) Cmax is between 5 and 15 μg/g, preferably 10 μg/g, and/or
(c) the drug pharmacokinetic profile overlaps with the kinetic profile for smooth muscle cell proliferation post-stent implantation, as depicted in FIG. 16B, preferably wherein the arterial tissue concentration at the site of stent implantation peaks between 15 and 25 days, preferably at 20 days, and then decreases to allow for vascular restoration; and
(2) the drug has a pharmacokinetic profile in the arterial tissue at the site of stent implantation about as depicted in FIG. 16A or FIG. 16B, optionally, wherein the drug is sirolimus.

29. A method of preparing a drug-eluting stent, wherein the drug-eluting stent achieves between 80% and 100% neointima strut coverage between day 20 and day 60 post-stent implantation comprising preparing a stent with the properties of the stent of claim 27.

30. The method of claim 29, wherein 80%-100% neointima strut coverage is achieved between day 20 and day 60 post-stent implantation.

31. The method of claim 29, wherein 80%-100% neointima strut coverage is achieved about 30 days post-stent implantation.
